SEQUENCE LISTING (160 sequences)

SEQ ID NO: 1 - protein, 510 aa, Mus musculus
MDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 2 - DNA, 12 bp, artificial sequence
  Test Polynucleotide p875
cagttaaaaact

SEQ ID NO: 3 - DNA, 11 bp, artificial sequence
  Test Polynucleotide p876
gagttaaaact

SEQ ID NO: 4 - DNA, 10 bp, artificial sequence
  Test Polynucleotide p877
cagcaaggct

SEQ ID NO: 5 - DNA, 18 bp, artificial sequence
  primer
aaaaaaaaaaaaaagggg

SEQ ID NO: 6 - protein, 25 aa, artificial sequence
  affinity tag for purifying TdT variants
MASSHHHHHHSSGSENLYFQTGSSG

SEQ ID NO: 7 - protein, 381 aa, Mus musculus
NSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 8 - protein, 380 aa, Unknown
  Bovine
DYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 9 - protein, 380 aa, Homo sapiens
DYSDSTNPGPPKTPPIAVQKISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 10 - protein, 376 aa, Gallus domesticus
QYPTLKTPESEVSSFTASKVSQYSCQRKTTLNNCNKKFTDAFEIMAENYEFKENEIFCLEFLRAASVLKSLPFPVTRMKDIQGLPCMGDRVRDVIEEIIEEGESSRAKDVLNDERYKSFKEFTSVFGVGVKTSEKWFRMGLRTVEEVKADKTLKLSKMQRAGFLYYEDLVSCVSKAEADAVSSIVKNTVCTFLPDALVTITGGFRRGKKIGHDIDFLITSPGQREDDELLHKGLLLYCDIIESTFVKEQIPSRHVDAMDHFQKCFAILKLYQPRVDNSSYNMSKKCDMAEVKDWKAIRVDLVITPFEQYAYALLGWTGSRQFGRDLRRYATHERKMMLDNHALYDKRKRVFLKAGSEEEIFAHLGLDYVEPWERNA

SEQ ID NO: 11 - protein, 387 aa, Didelphis marsupialis
SANPDPTAGTLNILPPTTKTISQYACQRRTTINNHNQRFTDAFEILAKNYEFKENDDTCLTFMRAISVLKCLPFEVVSLKDTEGLPWIGDEVKGIMEEIIEDGESLEVQAVLNDERYQSFKLFTSVFGVGLKTADKWYRMGFRTLNKIRSDKTLKLTKMQKAGLCYYEDLIDCVSKAEADAVSLLVQDAVWTFLPDALVTITGGFRRGKEFGHDVDFLITSPGAEKEQEDQLLQKVTNLWKKQGLLLYCDLIESTFEDLKLPSRKIDALDHFQKCFLILKLYHHKEDKRKWEMPTGSNESEAKSWKAIRVDLVVCPYDRYAFALLGWSGSRQFERDLRRYATHEKKMMLDNHALYDKTKKIFLKAKSEEEIFAHLGLEYIQPSERNA

SEQ ID NO: 12 - protein, 381 aa, Unknown
  shrew
DCPASHDSSPQKTESAAVQKISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 13 - protein, 387 aa, Unknown
  python
EKYQLPEDEDRSVTSDLDRDSISEYACQRRTTLKNYNQKFTDAFEILAENYEFNENKGFCTAFRRAASVLKCLPFTIVQVHDIEGVPWMGKQVKGIIEDIIEEGESSKVKAVLDNENYRSVKLFTSVFGVGLKTSDKWYRMGLRTLEEVKRDKNLKLTRMQKAGFLHYDDLTSCVSKAEADAASLIVQDVVWKIVPNAIVTIAGGFRRGKQTGHDVDFLITVPGSKQEEEELLHTVIDIWKKQELLLYYDLIESTFEDTKLPSRKVDALDHFQKCFAILKVHKEREDKGNSIRSKAFSEEEIKDWKAIRVDLVVVPFEQYAFALLGWTGSTQFERDLRRYATHEKKMMLDNHALYDKTKKIFLNAASEEEIFAHLGLDYLEPWERNA

SEQ ID NO: 14 - protein, 381 aa, Canis familiaris
DYTASPNPELQKTLPVAVKKISQYACQRRTTLNNYNNVFTDAFEVLAENYEFRENEVFSLTFMRAASVLKSLPFTIISMKDTEGIPCLGDQVKCIIEEIIEDGESSEVKAVLNDERYQSFKLFTSVFGVGLKTSEKWFRMGFRTLSKIKSDKSLKFTPMQKAGFLYYEDLVSCVTRAEAEAVGVLVKEAVGAFLPDAFVTMTGGFRRGKKMGHDVDFLITSPGSTDEDEEQLLPKVINLWERKGLLLYCDLVESTFEKLKLPSRKVDALDHFQKCFLILKLHHQRVDGGKCSQQEGKTWKAIRVDLVMCPYERRAFALLGWTGSRQFERDLRRYASHERKMILDNHALYDKTKKIFLKAESEEEIFAHLGLDYIEPWERNA

SEQ ID NO: 15 - protein, 382 aa, Unknown
  mole
GDCPASHDSSPQKTESAAVQKISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 16 - protein, 379 aa, Artificial Sequence
  artificial protein
EYSANPSPGPQATPAVYKISQYACQRRTTLNNHNHIFTDAFEILAENYEFKENEGCYVTYMRAASVLKSLPFTIVSMKDTEGIPCLEDKVKSIMEEIIEEGESSEVKAVLSDERYQCFKLFTSVFGVGLKTSEKWFRMGFRSLSNIRLDKSLKFTQMQKAGFRYYEDIVSCVTRAEAEAVDVLVNEAVRAFLPDAFITMTGGFRRGKKIGHDVDFLITSPELTEEDEQQLLHKVMNLWEKKGLLLYHDLVESTFEKLKQPSRKVDALDHFQKCFLIFKLYHERVGGDRCRQPEGKDWKAIRVDLVMCPYECHAFALLGWTGSRQFERDLRRYASHERKMILDNHALYDKTKRVFLQAENEEEIFAHLGLDYIEPWERNA

SEQ ID NO: 17 - protein, 384 aa, Erinaceus europaeus
DASFGSNPGSQNTPPLAIKKISQYACQRRTSLNNCNHIFTDALDILAENHEFRENEVSCVAFMRAASVLKSLPFTIISMKDTKGIPCLGDKAKCVIEEIIEDGESSEVKAILNDERYQSFKLFTSVFGVGLKTSEKWFRMGFRTLNKIMSDKTLKLTRMQKAGFLYYEDLVSCVAKAEADAVSVLVQEAVWAFLPDAMVTMTGGFRRGKKLGHDVDFLITSPGATEEEEQQLLPKVINFWERKGLLLYHDLVESTFEKLKLPSRKVDALDHFQKCFLILKLHLQHVNGVGNSKTGQQEGKNWKAIRVDLVMCPYERRAFALLGWTGSRQFERDLRRFATHERKMMLDNHALYDKTKRIFLKAESEEEIFAHLGLDYIDPWERNA

SEQ ID NO: 18 - protein, 381 aa, Unknown
  tree shrew
DHSTSPSPGPQKTPALAVQKISQYACQRRTTLNNCNRVFTDAFETLAENYEFRENEDSSVIFLRAASVLRSLPFTITSMRDTEGLPCLGDKVKCVIEEIIEDGESSEVNAVLNDERYKSFKLFTSVFGVGLKTSEKWFRMGFRTLSRVRSDKSLHLTRMQQAGFLYYEDLASCVTRAEAEAVGVLVKEAVGAFLPDALVTITGGFRRGKKTGHDVDFLITSPGSTEEKEEELLQKVLNLWEKKGLLLYYDLVESTFEKLKTPSRKVDALDHFPKCFLILKLHHQRVDGDKPSQQEGKSWKAIRVDLVMCPYERHAFALLGWTGSRQFERDLRRYATHERKMMLDNHALYDKTKRVFLKAESEEDIFAHLGLDYIEPWERNA

SEQ ID NO: 19 - protein, 394 aa, Ornithorhynchus anatinus
LTNSAPINCMTETPSLATKQVSQYACERRTTLNNCNQKFTDAFEILAKDFEFRENEGICLAFMRAISVLKCLPFTIVRMKDIEGVPWLGDQVKSIIEEIIEDGESSSVKAVLNDERYRSFQLFNSVFEVGLTDNGENGIARGFQTLNEVITDENISLTKTTLSTSLWNYLPGFLYYEDLVSCVAKEEADAVYLIVKEAVRAFLPEALVTLTGGFRRGKKIGHDVDFLISDPESGQDEQLLPNIIKLWEKQELLLYYDLVESTFEKTKIPSRKVDAMDHFQKCFLILKLHHQKVDSGRYKPPPESKNHEAKNWKAIRVDLVMCPFEQYAYALLGWTGSRQFERDLRRYATHEKKMMLDNHALYDKTKKIFLKAESEEDIFTHLGLDYIEPWERNA

SEQ ID NO: 20 - protein, 384 aa, Jaculus blanfordi
SSELELLDVSWLIECMGAGKPVEMTGRHQLVKQTFCLPGFILQDAFDILAENCEFRENEASCVEFMRAASVLKSLPFPIISVKDTEGIPWLGGKVKCVIEEIIEDGESSEVKALLNDERYKSFKLFTSVFGVGLKTAERWFRMGFRTLSTVKLDKSLTFTRMQKAGFLHYEDLVSCVTRAEAEAVSVLVQQAVVAFLPDALVSMTGGFRRGKKIGHDVDFLITSPEATEEEEQQLLHKVTNFWEQKGLLLYCDHVESTFEKCKLPSRKVDALDHFQKCFLILKLYRERVDSVKSSQQEGKGWKAIRVDLVMCPYECRAFALLGWTGSRQFERDLRRYATHERKMRLDNHALYDKTKRVFLKAESEEEIFAHLGLEYIEPLERNA

SEQ ID NO: 21 - protein, 364 aa, Unknown
  Puma
VVKKIPLYACQRRTTLNNFNHIFTDAFEVLAENYEFKENEISSATFMRAASVLKLPFTIISMKDTEGIPCLGDKVKCVIEEIIEDGESSEVKAVLNDERYQSFKLFTSVFGVGLKTSEKWFRMGFRTLSKIKSDKTLKFTQMQKAGFLYYEDLVSCVTRAEAEAVGVLVKEAVWAFLPDAFVTMTGGFRRGKKIGHDVDFLITIPGSTDEEEEQLLPKVINLWQRKELLLYYDLVESTFEKLKLPSRKVDALDHFQKCFLILKLHHQRVDSGKCSQQEGKTWKAIRVDLVMCPYERRAFALLGWTGSRQFERDLRRYATHERKMILDNHALYDKTKKIFLKAESEEEIFAHLGLDYIEPWERNA

SEQ ID NO: 22 - protein, 362 aa, Unknown
  reptilian
YACQRRTTLNNYNKKYTDAFEILAENYEMRENAGACLAFRRAASVLKFLPFAIVRSNDIEGLPWMGEQVKGIIEDILEEGQSPKVEAVLNNEKYRSFKLFTSVFGVALKTSEKWFMMGLRNLEDVKLNQNLQLTRMQKAGLQHYEDLISYVSKAEADSTSLMVKDTVWKFSPSALVTLTGGFRRGKKMGHDVDFLITVPGSRPNEELLHLVIDCWKKQGLLLYYDLIESTFEKSKLPSQRVDALDHFQKCFAILKLHKERVNQGTSLPPVASTVEEIKDWKAIRVDLVVSPFEQHAFALLGWTGSRQFERDLRRYATHEKKMMLDNHALYDKTKKIFLSASSEEEIFAHLGLDYLEPWERNA

SEQ ID NO: 23 - protein, 364 aa, Unknown
  shrew
VQKISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 24 - protein, 382 aa, Serinus canaria
SPPLNTPELEMPSFIATKVSQYSCQRKTTLNNYNKKFTDAFEVMAENYEFKENEIFCLEFLRAASLLKSLPFSVTRMKDIQGLPCMGDQVRDVIEIIEEGESSRVKEVLNDERYKAFKQFTSVFGVGVKTSEKWYRMGLRTVGEVKADKTLKLSKMQKAGFLYYEDLVSCVSKAEADAVSLIVKNTVCTFLPDALVTITGGFRRGKNIGHDIDFLITNPGPREDDELLHKVIDLWKKQGLLLYCDIIESTFVKEQLPSRKIDAMDHFQKCFAILKLYQPRVDNSTCNTSKKLEMAEVKDWKAIRVDLVITPFEQYSYALLGWTGSRQFGRDLRRYAAHERRMILDNHGLYDRTKRIFLKAGSEEEIFAHLGLDYVEPWERNA

SEQ ID NO: 25 - protein, 370 aa, Neopelma chrysocephalum
FIARKVSQYSCQRKTTLNNYNKKFTDAFEIMAENYEFKENEIFCLEFLRAASLLKYLPFPVTRMKDIQGLPCIGDQVRDVIEGIIEEGESSRVKEVLNDERYKAFKQFTSVFGVGVKTSEKWYRMGLRTVEELKADKTLKLSKMQKAGFLYYEDLVSCVSKAEADAVTLIVKNTVSTFLPDALVTITGGFRRGKKMGHDIDFLITNPGPREDDELLHKVVDLWKKQGLLLYCDIIESTFVEEQLPSRKVDAMDNFQKCFTILKLYQPGVDNSSYNMSKKSDMAEVKDWKAIRVDLVITPFEQYAYALLGWTGSREFGRDLRRYASHERKMILDNHGLYDRRKRIFLKAGSEEEIFAHLGLDYVEPWERNA

SEQ ID NO: 26 - protein, 383 aa, Alligator sinensis
SPSPVPGSQNVPAPSVDKVSQYACQRRTTLNNYNKKFTDAFEILAENCEFRENRLGCLEFLRAASVLKFLPFPIVKMKNIEGLPCMGDKVKCVIEEILEEGESCQAKEILNDERYKSFKLFTSVFGVGLKTTEKWYRMGFRTLEEVKAEKTLKLSRMQIAGFLHYEDIISYVSKAEADAVSLLIKDTVCMFLPDALVTITGGFRRGKKTGHDVDFLITNPGPEEEKELLHKVVDLWEKQGLLLYYDVIESTFEKEKRPSRKVDALDHFQKCFAILKLHQQRRGNSNSNISKESDKAEVKDWKAIRVDLVISPFEQYAYALLGWTGSRQFERDLRRYASRERKMMLDNHALYDKTKRTFLKAESEEEIFAHLGLDYIEPWERNA

SEQ ID NO: 27 - protein, 380 aa, Xenopus laevis
SPSPVPGSQNVPAPSEVKVSQYACQRCTTLQDTNRIFTDAFDILAEHFEFCENKGRTVAFLRASSLIKSLPFPITAMKELEGLPWLGDQMKGIIEEILEEGKSYKVLEVMNEERYKSFKQFTSVFGVGLKTSDKWFRMGFRTLEEIKNEKELKLTKMQKCGLLYYEDITSYVSRAEAETTEQLIKSIVWKFVPDAIVTLTGGFRRGKKKGHDVDILITCARKGKEKNILHNTMSVLKNRGLLLFYNIIESTFDETKLPSRHVDALDHFQKCFTILKLPKRQMDIGNIIDPHECERKNWKAVRLDLVITPYEQYPYALLGWTGSRQFERDLRRYATHEKRMMLDNHGLYDKTKNNFLKANNEEDIFKQLGLDYLEPWERNA

SEQ ID NO: 28 - protein, 383 aa, Notechis scutatus
SPSPVPGSQNVPAPSVDSISPYACQRRTTLKNYNQKFTDAFEILVENYEFNENKEFCLAFGRAASLLKCLPFTVVRVNDIEGLPWMGKQVREIIEDILEEGESSKVKAVLNDENYRSIKLFTSVFGVGLRTSEKWYRMGLRTLEEVKCNKNLTLTRMQKAGFFYYDDLISSVSKAEADAATQIVQDTVWKILPNAVVTLTGGFRRGKQTGHDVDFLITVPGSRQEEELLHPVIDIWKKQELLLYYDLIESTFENTKLPSRKVDALDHFQKCFAILKVHKERVNKGSAVQSNVFAEEGTKDWKAIRVDLVVTPFQHYAFALLGWTGSRQFERDLRRYATQEKKMMLDNHALYDKTKKIFLSAANEEEIFSHLGLDYLEPWERNA

SEQ ID NO: 29 - protein, 367 aa, Salmo truttal
TAAANVSQYACLRRTTTENHNKIFTDVLEELAENSEFNESKGPCLAFRRAASVLKSLPSAVHCLGAIQGLPCLGEHTKAVMEEILIFGRSFKVEEVQSDERYQALKLFTSVFGVGPKTAEKWYRRGLRSLKEILAEPNIQLNRMQRAGFLYYRDISKAVSKAEAKALSSIIEETAHWIAPDSILALTGGFRRGKEYGHDVDFLLTMPEMGKEEGLLLRVIDRLRDQGILLYCEHQDSTFDMSKLPSHRFEAMDHFEKCFLILRLEEGQVEGDGGLQKDPGESRGWRAVRVDLVAPPVDRYAFVLLGWTGSRQFERDLRRFASKERGMCLDNHALYDKTKKLFLPATSEEDIFAHLGLEYVEPWQRNA

SEQ ID NO: 30 - protein, 377 aa, Electrophorus electricus
SPSPVPGSQNVPAPSLPTVSQYACQRRTTLDNHNKVFTDALEVLIENYEFSDNKGACVGFRRAASVLKSLPKPLRCLKDMEGLPCLGDDTKAIIEEIYECGSSSRVENILSDEKYQTLKLFTSVFGVGPKTGEKWYRRGLRALEQVHSEPSIQLNKMQAAGFLYYEDISKPVSRAEAKAVGCIIEEVASCFSSSVTITLTGGFRRGKEFGHDVDFLLSIPEPGKEDGLLPAVIDRLRKQGILLYSDLQESTLQQWKRPSRCFDSMDHFQKCFLIVKLWTRLVEGHREDPSSQRDWKAVRVDLVVPPVDCYAFALLGWSGSTQFERDLRRFARLERRMLLDNHALYDKTTNTFLQAKTEEDIFAHLGLDYIEPWQRNA

SEQ ID NO: 31 - protein, 378 aa, Anabas testudineus
SPSPVPGSQNVPAPSVATVSQYACQRRTTTENNNKILTDAFEVLAESYELNQLEGPCLAFRRAASVLKSLPWAVQCLGATQGLPCLGEHTKALIEEILQYGHSFEVEKILSDERYQTLKLFTSVFGVGPKTAEKWYRRGLRSFSDILAEPSIQLNRMQQSGFLHYGDISRAVSKAEARALGNIIDEAVHAITPDGILALTGGFRRGKEFGHDVDFIVTTPEQGKEETLLPNIIDRLKEQGILLYSDYQTSTFDISKLPSHKFEAMDHFAKCFLILRLEGSLVDRGLNSTEGDSRGWRAVRVDLVSPPMERYAYALLGWTGSRQFERDLRRFARLEQHMLLDNHALYDKTKKEFLAATTERDIFAHLGLEYIEPWQRNA

SEQ ID NO: 32 - protein, 378 aa, Poecilia reticulate
SPSPVPGSQNVPAPSVDKVSQYACQRRTTVENNNRIFTDAFEVLAENYEFNEIEGRCLAFRRAASVLKSLPWAVRSVGATLDLPCLGEHTTAVMKEILQYGRSFEVEKILSDERCQTLKLFTSVFGVGPKTAEKWYRRGLRSFSDVLAQPGIHLNRMQQSGFLHYGDISRAVSKAEARAVGNIIDEAVHVITPNAILALTGGFRRGKDFGHDVDFIVTTTELGKEKNLLISVIESLKKQGLLLFSDYQASTFDISKLPSHRFEAMDHFAKCFLILRLEGSRVEGGLQRAQADGRGWRAVRVDLVSPPADRFAFTMLGWTGSRMFERDLRRFARLERQMLLDNHALYDKTKKEFLTAATEKDIFDHLGLEYIEPWQRNA

SEQ ID NO: 33 - protein, 366 aa, Rattus norvegicus
PLMQKISQYACQRRTTLNNHNQLFTDAFDILAENYEFRENEVSCLPFMRAASVLKSLSFPIVSMKDIEGIPCLGDKVKCVIEGIIEDGESSEVKAVLNDERYKSFKLFTSVFGVGLKTAEKWFRMGFRTLSKIKSDKSLRFTHMQKAGFLYYEDLVSCVNRAEAEAVSMLVKEAVVAFLPDALVTMTGGFRRGKMTGHDVDFLITSPEATEEEEQQLLHKVTNFWRQQGLLLYCDIIESTFEKFKLPSRKVDALDHFQKCFLILKLHRGLVRSEESGQQEGKDWKAIRVDLVMCPYERRAFALLGWTGSRQFERDLRRYATHERKMMLDNHALYDKTKRVFLEAESEEEIFAHLGLDYIEPWERNA

SEQ ID NO: 34 - protein, 379 aa, Rattus norvegicus
SLSPVPGSQTVPPPLMQKISQYACQRRTTLNNHNQLFTDAFDILAENYEFRENEVSCLPFMRAASVLKSLSFPIVSMKDIEGIPCLGDKVKCVIEGIIEDGESSEVKAVLNDERYKSFKLFTSVFGVGLKTAEKWFRMGFRTLSKIKSDKSLRFTHMQKAGFLYYEDLVSCVNRAEAEAVSMLVKEAVVAFLPDALVTMTGGFRRGKMTGHDVDFLITSPEATEEEEQQLLHKVTNFWRQQGLLLYCDIIESTFEKFKLPSRKVDALDHFQKCFLILKLHRGLVRSEESGQQEGKDWKAIRVDLVMCPYERRAFALLGWTGSRQFERDLRRYATHERKMMLDNHALYDKTKRVFLEAESEEEIFAHLGLDYIEPWERNA

SEQ ID NO: 35 - protein, 379 aa, Piliocolobus tephrosceles
SDSTNPGLPKTPPTAIQKISQYACQRRTTLNNCNQIFTDAFDILAENCEFRENEDSCVTFMRAASVLKSLPFTIISMKDTEGIPCLGSKVKCIIEEIIEDGESSEVKAVLNDERYQSFKLFTSVFGVGLKTSEKWFRMGFRTLSKVRSDESLKFTRMQRAGFLYYEDLVSCVTRAEAEAVSALVKEAVWAFLPDAFVTMTGGFRRGKKMGHDVDFLITSPGSTEDEEQQLLQKVMNLWEKKGLLLYYDLVESTFEKLRLPSRKVDALDHFQKCFLIFKLPLQRVDSDQSSWQGGKTWKAIRVDLVMCPYERRAFALLGWTGSRQFERDLRRYATHERKMILDNHALYDKTKRIFLKAESEEEIFAHLGLDYIEPWERNA

SEQ ID NO: 36 - protein, 379 aa, Sus scrofa
SASPSPGSQNTLPPAVKKISQYACQRRTTLNNCNHIFTDAFEVLAENYEFRENETFCLAFMRAASVLKSLPFTIISMKDTEGIPCLGDKVKCVIEEIIEDGESSEVKAVLNDERYQSFKLFTSVFGVGLKTSERWFRMGFRSLSKIRSDKTLKFTRMQKAGFLYYEDLVSCVTRAEAEAVGVLVKEAVQAFLPDAFVTMTGGFRRGKKMGHDVDFLITSPGSTDDEEQQLLPKVVNLWEREGLLLYCDLVESTLEKSKLPSRNVDALDHFQKCFLILKLHHQRVDSGMSSQQEGKTWKAIRVDLVMCPYELRAFALLGWTGSRQFERDLRRYATHERKMILDNHALYDKTKRIFLKAESEEEIFAHLGLDYLEPWERNA

SEQ ID NO: 37 - protein, 379 aa, Panthera tigris altaica
YSASPNPELQKTPPLVKKIPLYACQRRTTLNNFNHVFTDAFEVLAENYEFKENEVSSATFMRAASVLKSLPFTIISMKDTEGIPCLGDKVKCVIEEIIEDGESSEVKAVLNDERYQSFKLFTSVFGVGLKTSEKWFRMGFRTLSKIKSDKTLKFTQMQKAGFLYYEDLVSCVTRAEAEAVGVLVKEAVWAFLPDAFVTMTGGFRRGKKIGHDVDFLITSPGSTDEEEEELLPKVINLWQRKELLLYYDLVESTFEKLKLPSRKVDALDHFQKCFLILKLHHQRVDSGKCSQQEGKTWKAIRVDLVMCPYERRAFALLGWTGSRQFERDLRRYATHERKMILDNHALYDKTKKIFLKAESEEEIFAHLGLDYIEPWERNA

SEQ ID NO: 38 - protein, 365 aa, Bubalus bubalis
LAVKKISQYACQRKTTLNNYNHIFTDAFEILAENSEFKENEVSYVTFMRAASVLKSLPFTIISMKDTQGIPCLGDKVKCVIEEIIEDGESSEVKAVLNDERYQSFKLFTSVFGVGLKTSEKWFRMGFRSLSKITSDKTLKFTKMQKAGFLYYEDLVSCVTRAEAEAVGVLVKEAVWAFLPDAFITMTGGFRRGKKIGHDVDFLITSPGSAEDEEQLLPKVINLWEKKGLLLYYDLVESTFEKFKLPSRQVDTLDHFQKCFLILKLHHQRVDSGRSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 39 - protein, 379 aa, Marmota flaviventris
PDHSNSDPQRIPPPAVQTISQYACQRRTTLNNCNRVFTDAFDVLAENYEFRENESCSVVFMRAASVLKSLPFTIISMRDLEGIPCLEGKAKSIIEEIIEDGESSEVKAVLNDERYKSFKLFTSVFGVGLKTSEKWFRMGFRTLSKIRSDKSLKFTHMQKAGFLYYEDLVSCVTRAEAEAVSVLVKEAVWAFLPDAFITMTGGFRRGKNIGHDVDFLITSAEATEEEEQQLLHKVTNLWEKKGLLLYCDLVESTFEKLKTPSRKVDALDHFQKCFLILKLHHQRVDSDKASQQGGKNWKAIRVDLVMCPYERRAFALLGWTGSRQFERDLRRYATHERKMILDNHALYDKTKRIFLKAESEEEIFAHLGLDYIEPWERNA

SEQ ID NO: 40 - protein, 362 aa, Mus musculus
KISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 41 - protein, 361 aa, Unknown
  Bovine
KISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 42 - protein, 361 aa, Homo sapiens
KISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 43 - protein, 358 aa, Gallus domesticus
KVSQYSCQRKTTLNNCNKKFTDAFEIMAENYEFKENEIFCLEFLRAASVLKSLPFPVTRMKDIQGLPCMGDRVRDVIEEIIEEGESSRAKDVLNDERYKSFKEFTSVFGVGVKTSEKWFRMGLRTVEEVKADKTLKLSKMQRAGFLYYEDLVSCVSKAEADAVSSIVKNTVCTFLPDALVTITGGFRRGKKIGHDIDFLITSPGQREDDELLHKGLLLYCDIIESTFVKEQIPSRHVDAMDHFQKCFAILKLYQPRVDNSSYNMSKKCDMAEVKDWKAIRVDLVITPFEQYAYALLGWTGSRQFGRDLRRYATHERKMMLDNHALYDKRKRVFLKAGSEEEIFAHLGLDYVEPWERNA

SEQ ID NO: 44 - protein, 368 aa, Didelphis marsupialis
TISQYACQRRTTINNHNQRFTDAFEILAKNYEFKENDDTCLTFMRAISVLKCLPFEVVSLKDTEGLPWIGDEVKGIMEEIIEDGESLEVQAVLNDERYQSFKLFTSVFGVGLKTADKWYRMGFRTLNKIRSDKTLKLTKMQKAGLCYYEDLIDCVSKAEADAVSLLVQDAVWTFLPDALVTITGGFRRGKEFGHDVDFLITSPGAEKEQEDQLLQKVTNLWKKQGLLLYCDLIESTFEDLKLPSRKIDALDHFQKCFLILKLYHHKEDKRKWEMPTGSNESEAKSWKAIRVDLVVCPYDRYAFALLGWSGSRQFERDLRRYATHEKKMMLDNHALYDKTKKIFLKAKSEEEIFAHLGLEYIQPSERNA

SEQ ID NO: 45 - protein, 362 aa, Unknown
  shrew
KISQYACQRRTTLNNHNHIFTDAFEILAENCEFRENEGSYVTYMRAASVLKSLPFSIISMKDTEGIPCLADKVKCVIEEIIEDGESSEVKAVLNDERYKSFKLFTSVFGVGLKTAEKWFRLGFRTLSGIMNDKTLKLTHMQKAGFLYYEDLVSCVTRAEAEAVGVLVKEAVWAFLPDAIVTMTGGFRRGKKVGHDVDFLITSPEATEEQEQQLLHKVITFWEKEGLLLYCDLYESTFEKLKMPSRKVDALDHFQKCFLILKLHRECVDDGTSSQLQGKTWKAIRVDLVVCPYECRAFALLGWTGSPQFERDLRRYATHERKMMLDNHALYDKTKRKFLSADSEEDIFAHLGLDYIEPWERNA

SEQ ID NO: 46 - protein, 362 aa, Canis familiaris
KISQYACQRRTTLNNYNNVFTDAFEVLAENYEFRENEVFSLTFMRAASVLKSLPFTIISMKDTEGIPCLGDQVKCIIEEIIEDGESSEVKAVLNDERYQSFKLFTSVFGVGLKTSEKWFRMGFRTLSKIKSDKSLKFTPMQKAGFLYYEDLVSCVTRAEAEAVGVLVKEAVGAFLPDAFVTMTGGFRRGKKMGHDVDFLITSPGSTDEDEEQLLPKVINLWERKGLLLYCDLVESTFEKLKLPSRKVDALDHFQKCFLILKLHHQRVDGGKCSQQEGKTWKAIRVDLVMCPYERRAFALLGWTGSRQFERDLRRYASHERKMILDNHALYDKTKKIFLKAESEEEIFAHLGLDYIEPWERNA

SEQ ID NO: 47 - protein, 362 aa, Unknown; identical to SEQ ID NO: 45
  mole

SEQ ID NO: 48 - protein, 362 aa, Ochotona princeps
KISQYACQRRTTLNNHNHIFTDAFEILAENYEFKENEGCYVTYMRAASVLKSLPFTIVSMKDTEGIPCLEDKVKSIMEEIIEEGESSEVKAVLSDERYQCFKLFTSVFGVGLKTSEKWFRMGFRSLSNIRLDKSLKFTQMQKAGFRYYEDIVSCVTRAEAEAVDVLVNEAVRAFLPDAFITMTGGFRRGKKIGHDVDFLITSPELTEEDEQQLLHKVMNLWEKKGLLLYHDLVESTFEKLKQPSRKVDALDHFQKCFLIFKLYHERVGGDRCRQPEGKDWKAIRVDLVMCPYECHAFALLGWTGSRQFERDLRRYASHERKMILDNHALYDKTKRVFLQAENEEEIFAHLGLDYIEPWERNA

SEQ ID NO: 49 - protein, 365 aa, Erinaceus europaeus
KISQYACQRRTSLNNCNHIFTDALDILAENHEFRENEVSCVAFMRAASVLKSLPFTIISMKDTKGIPCLGDKAKCVIEEIIEDGESSEVKAILNDERYQSFKLFTSVFGVGLKTSEKWFRMGFRTLNKIMSDKTLKLTRMQKAGFLYYEDLVSCVAKAEADAVSVLVQEAVWAFLPDAMVTMTGGFRRGKKLGHDVDFLITSPGATEEEEQQLLPKVINFWERKGLLLYHDLVESTFEKLKLPSRKVDALDHFQKCFLILKLHLQHVNGVGNSKTGQQEGKNWKAIRVDLVMCPYERRAFALLGWTGSRQFERDLRRFATHERKMMLDNHALYDKTKRIFLKAESEEEIFAHLGLDYIDPWERNA

SEQ ID NO: 50 - protein, 362 aa, Unknown
  tree shrew
KISQYACQRRTTLNNCNRVFTDAFETLAENYEFRENEDSSVIFLRAASVLRSLPFTITSMRDTEGLPCLGDKVKCVIEEIIEDGESSEVNAVLNDERYKSFKLFTSVFGVGLKTSEKWFRMGFRTLSRVRSDKSLHLTRMQQAGFLYYEDLASCVTRAEAEAVGVLVKEAVGAFLPDALVTITGGFRRGKKTGHDVDFLITSPGSTEEKEEELLQKVLNLWEKKGLLLYYDLVESTFEKLKTPSRKVDALDHFPKCFLILKLHHQRVDGDKPSQQEGKSWKAIRVDLVMCPYERHAFALLGWTGSRQFERDLRRYATHERKMMLDNHALYDKTKRVFLKAESEEDIFAHLGLDYIEPWERNA

SEQ ID NO: 51 - protein, 375 aa, Ornithorhynchus anatinus
QVSQYACERRTTLNNCNQKFTDAFEILAKDFEFRENEGICLAFMRAISVLKCLPFTIVRMKDIEGVPWLGDQVKSIIEEIIEDGESSSVKAVLNDERYRSFQLFNSVFEVGLTDNGENGIARGFQTLNEVITDENISLTKTTLSTSLWNYLPGFLYYEDLVSCVAKEEADAVYLIVKEAVRAFLPEALVTLTGGFRRGKKIGHDVDFLISDPESGQDEQLLPNIIKLWEKQELLLYYDLVESTFEKTKIPSRKVDAMDHFQKCFLILKLHHQKVDSGRYKPPPESKNHEAKNWKAIRVDLVMCPFEQYAYALLGWTGSRQFERDLRRYATHEKKMMLDNHALYDKTKKIFLKAESEEDIFTHLGLDYIEPWERNA

SEQ ID NO: 52 - protein, 365 aa, Serinus canaria
KVSQYSCQRKTTLNNYNKKFTDAFEVMAENYEFKENEIFCLEFLRAASLLKSLPFSVTRMKDIQGLPCMGDQVRDVIEIIEEGESSRVKEVLNDERYKAFKQFTSVFGVGVKTSEKWYRMGLRTVGEVKADKTLKLSKMQKAGFLYYEDLVSCVSKAEADAVSLIVKNTVCTFLPDALVTITGGFRRGKNIGHDIDFLITNPGPREDDELLHKVIDLWKKQGLLLYCDIIESTFVKEQLPSRKIDAMDHFQKCFAILKLYQPRVDNSTCNTSKKLEMAEVKDWKAIRVDLVITPFEQYSYALLGWTGSRQFGRDLRRYAAHERRMILDNHGLYDRTKRIFLKAGSEEEIFAHLGLDYVEPWERNA

SEQ ID NO: 53 - protein, 366 aa, Neopelma chrysocephalum
KVSQYSCQRKTTLNNYNKKFTDAFEIMAENYEFKENEIFCLEFLRAASLLKYLPFPVTRMKDIQGLPCIGDQVRDVIEGIIEEGESSRVKEVLNDERYKAFKQFTSVFGVGVKTSEKWYRMGLRTVEELKADKTLKLSKMQKAGFLYYEDLVSCVSKAEADAVTLIVKNTVSTFLPDALVTITGGFRRGKKMGHDIDFLITNPGPREDDELLHKVVDLWKKQGLLLYCDIIESTFVEEQLPSRKVDAMDNFQKCFTILKLYQPGVDNSSYNMSKKSDMAEVKDWKAIRVDLVITPFEQYAYALLGWTGSREFGRDLRRYASHERKMILDNHGLYDRRKRIFLKAGSEEEIFAHLGLDYVEPWERNA

SEQ ID NO: 54 - protein, 366 aa, Alligator sinensis
KVSQYACQRRTTLNNYNKKFTDAFEILAENCEFRENRLGCLEFLRAASVLKFLPFPIVKMKNIEGLPCMGDKVKCVIEEILEEGESCQAKEILNDERYKSFKLFTSVFGVGLKTTEKWYRMGFRTLEEVKAEKTLKLSRMQIAGFLHYEDIISYVSKAEADAVSLLIKDTVCMFLPDALVTITGGFRRGKKTGHDVDFLITNPGPEEEKELLHKVVDLWEKQGLLLYYDVIESTFEKEKRPSRKVDALDHFQKCFAILKLHQQRRGNSNSNISKESDKAEVKDWKAIRVDLVISPFEQYAYALLGWTGSRQFERDLRRYASRERKMMLDNHALYDKTKRTFLKAESEEEIFAHLGLDYIEPWERNA

SEQ ID NO: 55 - protein, 366 aa, Artificial Sequence
  trunc new
KISQYACQRRTTLNNYNKKFTDAFEILAENYEFNENKGFCLAFRRAASVLKFLPFTIVRMNDIQGLPWMGDQVKRVIEEILEEGESSKVKEVLNDEKYKAFKLFTSVFGVGRKTSEKWYRMGLRTLEEVKADKTLKLTKMQKAGFLYYEDLISRVSKAEADAVSLIVKDTVWKFLPDALVTLTGGFRLGKKMGHDVDFLITTPGPEEEEELLHKVIDLWKKQGLLLYYDIIESTFEKTKLPSRTVDALDHFQKCFAILKLHKQRVDNGNSNQSKEFDKEETKDWKAIRVDLVITPFEQYAYALLGWTGSAFFNRDLRRYATHEKKMMLDNHALYDKTKRIFLKAASEEEIFAHLGLDYLEPWERNA

SEQ ID NO: 56 - protein, 363 aa, Xenopus laevis
KVSQYACQRCTTLQDTNRIFTDAFDILAEHFEFCENKGRTVAFLRASSLIKSLPFPITAMKELEGLPWLGDQMKGIIEEILEEGKSYKVLEVMNEERYKSFKQFTSVFGVGLKTSDKWFRMGFRTLEEIKNEKELKLTKMQKCGLLYYEDITSYVSRAEAETTEQLIKSIVWKFVPDAIVTLTGGFRRGKKKGHDVDILITCARKGKEKNILHNTMSVLKNRGLLLFYNIIESTFDETKLPSRHVDALDHFQKCFTILKLPKRQMDIGNIIDPHECERKNWKAVRLDLVITPYEQYPYALLGWTGSRQFERDLRRYATHEKRMMLDNHGLYDKTKNNFLKANNEEDIFKQLGLDYLEPWERNA

SEQ ID NO: 57 - protein, 363 aa, Unknown
  Xenopus sp.
KVSQYACQRRTTLQDTNRIFTDAFDILAENYEFNENKGPCVAFRRASSVLKSLPFPIVAMKDLEGLPWLGDQMKRIIEEILEEGKSSKVVEVMNDERYKAFKLFTSVFGVGRKTSEKWFRMGFRTLEEIKSDKELKLTKMQKAGLLYYEDITSAVSKAEAEAVEQLIKSIVWKFVPDAIVTLTGGFRLGKKMGHDVDILITTPRKGEKEEILHKTINVLKKRGLLLFYNIIESTFDETKLPSRHVDALDHFQKCFTILKLQKQQLDNGNSNEPFETEKKNWKAIRVDLVITPYEQYAYALLGWTGSPQFNRDLRRYATHEKRMMLDNHALYDKTKNIFLKANSEEDIFTHLGLDYLEPWERNA

SEQ ID NO: 58 - protein, 367 aa, Unknown
  Notechis sp.
SISQYACQRRTTLNNYNKKFTDAFEILAENYEFNENKGFCLAFRRAASVLKFLPFTIVRVNDIEGLPWMGEQVKRIIEDILEEGESSKVKAVLNNENYRAFKLFTSVFGVGRKTSEKWYRMGLRTLEEVKADKNLKLTRMQKAGFLHYEDLISRVSKAEADAASLIVKDTVWKFLPNALVTLTGGFRLGKKMGHDVDFLITVPGSRQEEEELLHTVIDLWKKQGLLLYYDLIESTFEKTKLPSRTVDALDHFQKCFAILKLHKERVDKGNSIQSKAFAEEETKDWKAIRVDLVVTPFEQYAFALLGWTGSPFFNRDLRRYATHEKKMMLDNHALYDKTKKIFLKAASEEEIFAHLGLDYLEPWERNA

SEQ ID NO: 59 - protein, 363 aa, Salmo trutta
NVSQYACLRRTTTENHNKIFTDVLEELAENSEFNESKGPCLAFRRAASVLKSLPSAVHCLGAIQGLPCLGEHTKAVMEEILIFGRSFKVEEVQSDERYQALKLFTSVFGVGPKTAEKWYRRGLRSLKEILAEPNIQLNRMQRAGFLYYRDISKAVSKAEAKALSSIIEETAHWIAPDSILALTGGFRRGKEYGHDVDFLLTMPEMGKEEGLLLRVIDRLRDQGILLYCEHQDSTFDMSKLPSHRFEAMDHFEKCFLILRLEEGQVEGDGGLQKDPGESRGWRAVRVDLVAPPVDRYAFVLLGWTGSRQFERDLRRFASKERGMCLDNHALYDKTKKLFLPATSEEDIFAHLGLEYVEPWQRNA

SEQ ID NO: 60 - protein, 363 aa, Artificial Sequence
  trunc new
TVSQYACQRRTTMENHNKIFTDAFEVLAENSEFNESKESRDAFRRAASVLKSLPSAVRSLGDTEGLPCLGEHTKRVIEEILEYGRSSKVEDILSDERYQTMKLFTSVFGVGPKTAEKWYRRGLRSLEEVRAEPSIHLNRMQRAGFLYYRDISKRVSKAEAKALGNIIEETVHAIAPDAIVTLTGGFRLGKEFGHDVDFILTTPEMGKEEGLLPRVIDRLRNQGILLYCDYQESTFDMSKLPSRTFEAMDHFQKCFLIIKLKEGLVEGEGGLQSDPGDRRGWRAVRVDLVAPPVDRYAFALLGWTGSPQFNRDLRRFARLERRMLLDNHALYDKTKKIFLPAKTEEDIFAHLGLEYIEPWQRNA

SEQ ID NO: 61 - protein, 360 aa, Electrophorus electricus
TVSQYACQRRTTLDNHNKVFTDALEVLIENYEFSDNKGACVGFRRAASVLKSLPKPLRCLKDMEGLPCLGDDTKAIIEEIYECGSSSRVENILSDEKYQTLKLFTSVFGVGPKTGEKWYRRGLRALEQVHSEPSIQLNKMQAAGFLYYEDISKPVSRAEAKAVGCIIEEVASCFSSSVTITLTGGFRRGKEFGHDVDFLLSIPEPGKEDGLLPAVIDRLRKQGILLYSDLQESTLQQWKRPSRCFDSMDHFQKCFLIVKLWTRLVEGHREDPSSQRDWKAVRVDLVVPPVDCYAFALLGWSGSTQFERDLRRFARLERRMLLDNHALYDKTTNTFLQAKTEEDIFAHLGLDYIEPWQRNA

SEQ ID NO: 62 - protein, 360 aa, Artificial Sequence
  trunc new
TVSQYACQRRTTLDNHNKIFTDALEVLAENYEFSDSKESRDAFRRAASVLKSLPTPLRSLQDTEGLPCLGEETKRVIEEIFEEGSSSRVEDILSDERYQTLKLFTSVFGVGPKTAEKWYRRGLRSLEQVRSDPSIHLNRMQTAGFLYYEDISKPVSKAEAKALGNIIEETASAFSPSVTITLTGGFRLGKEFGHDVDFLLTVPEPGKEDGLLPAVIDRLRSQGILLYSDFQESTFDLSKLPSRRFEAMDHFQKCFLIVKLKAGLVEGEGADPGDRRDWRAVRVDLVAPPVDRYAFALLGWSGSAFFNRDLRRFARLERGMLLDNHALYDKTTNTFLQAKTEEDIFAHLGLDYIEPWQRNA

SEQ ID NO: 63 - protein, 361 aa, Anabas testudineus
TVSQYACQRRTTTENNNKILTDAFEVLAESYELNQLEGPCLAFRRAASVLKSLPWAVQCLGATQGLPCLGEHTKALIEEILQYGHSFEVEKILSDERYQTLKLFTSVFGVGPKTAEKWYRRGLRSFSDILAEPSIQLNRMQQSGFLHYGDISRAVSKAEARALGNIIDEAVHAITPDGILALTGGFRRGKEFGHDVDFIVTTPEQGKEETLLPNIIDRLKEQGILLYSDYQTSTFDISKLPSHKFEAMDHFAKCFLILRLEGSLVDRGLNSTEGDSRGWRAVRVDLVSPPMERYAYALLGWTGSRQFERDLRRFARLEQHMLLDNHALYDKTKKEFLAATTERDIFAHLGLEYIEPWQRNA

SEQ ID NO: 64 - protein, 361 aa, Unknown
  walking fish
TVSQYACQRRTTTENNNKIFTDAFEVLAESYEFNEMEESRDAFRRAASVLKSLPWAVQSLGATQDLPCLGEHTKRVMEEILQYGRSFEVEKILSDERYQTLKLFTSVFGVGPKTAEKWYRRGLRSFEDIRAEPSIHLNRMQQSGFLHYGDISRAVSKAEARALGNIIDEAVHAITPDAILALTGGFRLGKEFGHDVDFIVTTPELGKEESLLPNIIDRLKKQGILLYSDYQASTFDMSKLPSHRFEAMDHFAKCFLILRLEGSQVEGGLNSAEGDSRGWRAVRVDLVSPPMDRYAFALLGWTGSAFFNRDLRRFARMERRMLLDNHALYDKTKKEFLAATTEKDIFAHLGLEYIEPWQRNA

SEQ ID NO: 65 - protein, 361 aa, Poecilia reticulate
KVSQYACQRRTTVENNNRIFTDAFEVLAENYEFNEIEGRCLAFRRAASVLKSLPWAVRSVGATLDLPCLGEHTTAVMKEILQYGRSFEVEKILSDERCQTLKLFTSVFGVGPKTAEKWYRRGLRSFSDVLAQPGIHLNRMQQSGFLHYGDISRAVSKAEARAVGNIIDEAVHVITPNAILALTGGFRRGKDFGHDVDFIVTTTELGKEKNLLISVIESLKKQGLLLFSDYQASTFDISKLPSHRFEAMDHFAKCFLILRLEGSRVEGGLQRAQADGRGWRAVRVDLVSPPADRFAFTMLGWTGSRMFERDLRRFARLERQMLLDNHALYDKTKKEFLTAATEKDIFDHLGLEYIEPWQRNA

SEQ ID NO: 66 - protein, 366 aa, Artificial Sequence; identical to SEQ ID NO: 55
  F57 trunc new (Poecilia reticulate) guppy [56.66 percent identity to wt calc by NCBI BLASTP]

SEQ ID NO: 67 - protein, 362 aa, Rattus norvegicus
KISQYACQRRTTLNNHNQLFTDAFDILAENYEFRENEVSCLPFMRAASVLKSLSFPIVSMKDIEGIPCLGDKVKCVIEGIIEDGESSEVKAVLNDERYKSFKLFTSVFGVGLKTAEKWFRMGFRTLSKIKSDKSLRFTHMQKAGFLYYEDLVSCVNRAEAEAVSMLVKEAVVAFLPDALVTMTGGFRRGKMTGHDVDFLITSPEATEEEEQQLLHKVTNFWRQQGLLLYCDIIESTFEKFKLPSRKVDALDHFQKCFLILKLHRGLVRSEESGQQEGKDWKAIRVDLVMCPYERRAFALLGWTGSRQFERDLRRYATHERKMMLDNHALYDKTKRVFLEAESEEEIFAHLGLDYIEPWERNA

SEQ ID NO: 68 - protein, 366 aa, artificial sequence
  N100 trunc new
SVVQKISEYACQRRTTLNNHNEVFTRAFDILAENYEFRENEESRDAFRRASSVLKSLPFPIISMKDTEGIPCLGDKVKRVIEEIIEDGESSEVKAVLNDERYKAFKLFTSVFGVGRKTAEKWFRMGFRTLEKIKSDKSLRFTQMQKAGFLYYEDLAERVNRAEAEAIEMLVKEAVVAFLPDALVTMTGGFRLGKMTGHDVDFLITSPEATEEEEQQLLHKVTDFWRQQGLLLYCDIIESTFEKFKLPSRTVDALDHFQKCFLILKLHHGRVVSEKSGQQEGKGWKAIRVDLVMCPYERRAFALLGWTGSPFFNRDLRRYATHERKMMLDNHALYDKTKRVFLEAESEEEIFAHLGLDYIEPWERNA

SEQ ID NO: 69 - protein, 362 aa, Piliocolobus tephrosceles
KISQYACQRRTTLNNCNQIFTDAFDILAENCEFRENEDSCVTFMRAASVLKSLPFTIISMKDTEGIPCLGSKVKCIIEEIIEDGESSEVKAVLNDERYQSFKLFTSVFGVGLKTSEKWFRMGFRTLSKVRSDESLKFTRMQRAGFLYYEDLVSCVTRAEAEAVSALVKEAVWAFLPDAFVTMTGGFRRGKKMGHDVDFLITSPGSTEDEEQQLLQKVMNLWEKKGLLLYYDLVESTFEKLRLPSRKVDALDHFQKCFLIFKLPLQRVDSDQSSWQGGKTWKAIRVDLVMCPYERRAFALLGWTGSRQFERDLRRYATHERKMILDNHALYDKTKRIFLKAESEEEIFAHLGLDYIEPWERNA

SEQ ID NO: 70 - protein, 362 aa, Sus domesticus
KISQYACQRRTTLNNCNHIFTDAFEVLAENYEFRENETFCLAFMRAASVLKSLPFTIISMKDTEGIPCLGDKVKCVIEEIIEDGESSEVKAVLNDERYQSFKLFTSVFGVGLKTSERWFRMGFRSLSKIRSDKTLKFTRMQKAGFLYYEDLVSCVTRAEAEAVGVLVKEAVQAFLPDAFVTMTGGFRRGKKMGHDVDFLITSPGSTDDEEQQLLPKVVNLWEREGLLLYCDLVESTLEKSKLPSRNVDALDHFQKCFLILKLHHQRVDSGMSSQQEGKTWKAIRVDLVMCPYELRAFALLGWTGSRQFERDLRRYATHERKMILDNHALYDKTKRIFLKAESEEEIFAHLGLDYLEPWERNA

SEQ ID NO: 71 - protein, 362 aa, artificial sequence
  N27 trunc new  [88.13 percent identity to wt calc by NCBI BLASTP] pig
KISQYACQRRTTLNNYNEIFTRAFEILAENYEFRENEESYVTFRRAASVLKSLPFTIISMKDTEGIPCLGDKVKRVIEEIIEDGESSEVKAVLNDERYKAFKLFTSVFGVGRKTSEKWFRMGFRTLEKIRSDKTLKFTRMQKAGFLYYEDLVSRVTRAEAEAVSVLVKEAVWAFLPDAFVTMTGGFRLGKKIGHDVDFLITSPGSTEEEEQQLLHKVINLWEKKGLLLYYDLVESTFEKLKLPSRTVDALDHFQKCFLILKLHHQRVGSGKSSQQEGKTWKAIRVDLVMCPYERRAFALLGWTGSPQFNRDLRRYATHERKMMLDNHALYDKTKRIFLKAESEEEIFAHLGLDYIEPWERNA

SEQ ID NO: 72 - protein, 362 aa, artificial sequence
  N35 trunc new
KISQYACQRRTTLNNYNEIFTRAFEVLAENYEFRENEESYVTFRRAASVLKSLPFTIISMKDTEGIPCLGDKVKRVIEEIIEDGESSEVKAVLNDERYQAFKLFTSVFGVGRKTSEKWFRMGFRTLEKIRSDKTLKFTRMQKAGFLYYEDLAERVTRAEAEAVSVLVKEAVWAFLPDAFVTMTGGFRLGKKIGHDVDFLITSPGSTEEEEQQLLPKVINLWERKGLLLYYDLVESTFEKLKLPSRKVDALDHFQKCFLILKLHHQRVGSGKSSQQEGKTWKAIRVDLVMCPYERRAFALLGWTGSAFFNRDLRRYATHERKMILDNHALYDKTKRIFLKAESEEEIFAHLGLDYIEPWERNA

SEQ ID NO: 73 - protein, 361 aa, Bubalus bubalis
KISQYACQRKTTLNNYNHIFTDAFEILAENSEFKENEVSYVTFMRAASVLKSLPFTIISMKDTQGIPCLGDKVKCVIEEIIEDGESSEVKAVLNDERYQSFKLFTSVFGVGLKTSEKWFRMGFRSLSKITSDKTLKFTKMQKAGFLYYEDLVSCVTRAEAEAVGVLVKEAVWAFLPDAFITMTGGFRRGKKIGHDVDFLITSPGSAEDEEQLLPKVINLWEKKGLLLYYDLVESTFEKFKLPSRQVDTLDHFQKCFLILKLHHQRVDSGRSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 74 - protein, 362 aa, Marmota flaviventris
TISQYACQRRTTLNNCNRVFTDAFDVLAENYEFRENESCSVVFMRAASVLKSLPFTIISMRDLEGIPCLEGKAKSIIEEIIEDGESSEVKAVLNDERYKSFKLFTSVFGVGLKTSEKWFRMGFRTLSKIRSDKSLKFTHMQKAGFLYYEDLVSCVTRAEAEAVSVLVKEAVWAFLPDAFITMTGGFRRGKNIGHDVDFLITSAEATEEEEQQLLHKVTNLWEKKGLLLYCDLVESTFEKLKTPSRKVDALDHFQKCFLILKLHHQRVDSDKASQQGGKNWKAIRVDLVMCPYERRAFALLGWTGSRQFERDLRRYATHERKMILDNHALYDKTKRIFLKAESEEEIFAHLGLDYIEPWERNA

SEQ ID NO: 75 - protein, 362 aa, artificial sequence
  Truncated mouse TdT M53
KISQYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLAERVNRPEAEAIRMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSPQFNRDLRRYATHERKMMLDNHALYDKTKRVFLEAESEEEIFAHLGLDYIEPWERNA

SEQ ID NO: 76 - protein, 379 aa, artificial sequence
  N93or trunc new  [95.15 percent identity to wt (Marmota flaviventris)calc by NCBI BLASTP]
SPSPVPGSQNVPAPAVQTISQYACQRRTTLNNYNRVFTDAFDVLAENYEFRENEESSVVFRRAASVLKSLPFTIISMRDLEGIPCLEGKAKRIIEEIIEDGESSEVKAVLNDERYKAFKLFTSVFGVGRKTSEKWFRMGFRTLEKIRSDKSLKFTHMQKAGFLYYEDLVSRVTRAEAEAVSVLVKEAVWAFLPDAFITMTGGFRLGKNIGHDVDFLITSAEATEEEEQQLLHKVTNLWEKKGLLLYCDLVESTFEKLKLPSRTVDALDHFQKCFLILKLHHQRVDSDKASQQGGKNWKAIRVDLVMCPYERRAFALLGWTGSPQFNRDLRRYATHERKMILDNHALYDKTKRIFLKAESEEEIFAHLGLDYIEPRERNA

SEQ ID NO: 77 - protein, 366 aa, artificial sequence
  N72 trunc new  [91.76 percent identity to wt(Bubalus bubalis) calc by NCBI BLASTP]
SVVKKISQYACQRRTTLNNYNEIFTRAFEILAENYEFKENEESYVTFRRAASVLKSLPFTIISMKDTEGIPCLGDKVKRVIEEIIEDGESSEVKAVLNDERYQAFKLFTSVFGVGRKTSEKWFRMGFRSLEKIRSDKTLKFTRMQKAGFLYYEDLVSRVTRAEAEAVGVLVKEAVWAFLPDAFVTMTGGFRLGKKIGHDVDFLITSPGSTEDEEQQLLPKVINLWERKGLLLYYDLVESTFEKFKLPSRQVDALDHFQKCFLILKLHHQRVGSGKSSQQEGKTWKAIRVDLVMCPYEQRAFALLGWTGSAFFNRDLRRYATHERKMMLDNHALYDKTKRIFLKAESEEEIFAHLGLDYIEPRERNA

SEQ ID NO: 78 - protein, 379 aa, artificial sequence
  N35 trunc new  [89.18 percent identity to wt (Panthera tigris altaica) calc by NCBI BLASTP]
SPSPVPGSQNVPAPVVKKISQYACQRRTTLNNYNEIFTRAFEVLAENYEFRENEESYVTFRRAASVLKSLPFTIISMKDTEGIPCLGDKVKRVIEEIIEDGESSEVKAVLNDERYQAFKLFTSVFGVGRKTSEKWFRMGFRTLEKIRSDKTLKFTRMQKAGFLYYEDLAERVTRAEAEAVSVLVKEAVWAFLPDAFVTMTGGFRLGKKIGHDVDFLITSPGSTEEEEQQLLPKVINLWERKGLLLYYDLVESTFEKLKLPSRKVDALDHFQKCFLILKLHHQRVGSGKSSQQEGKTWKAIRVDLVMCPYERRAFALLGWTGSAFFNRDLRRYATHERKMILDNHALYDKTKRIFLKAESEEEIFAHLGLDYIEPWERNA

SEQ ID NO: 79 - protein, 379 aa, artificial sequence
  N27 trunc new  [88.13 percent identity to wt(Sus scrofa) calc by NCBI BLASTP]
SPSPVPGSQNVPAPVVKKISQYACQRRTTLNNYNEIFTRAFEILAENYEFRENEESYVTFRRAASVLKSLPFTIISMKDTEGIPCLGDKVKRVIEEIIEDGESSEVKAVLNDERYKAFKLFTSVFGVGRKTSEKWFRMGFRTLEKIRSDKTLKFTRMQKAGFLYYEDLVSRVTRAEAEAVSVLVKEAVWAFLPDAFVTMTGGFRLGKKIGHDVDFLITSPGSTEEEEQQLLHKVINLWEKKGLLLYYDLVESTFEKLKLPSRTVDALDHFQKCFLILKLHHQRVGSGKSSQQEGKTWKAIRVDLVMCPYERRAFALLGWTGSPQFNRDLRRYATHERKMMLDNHALYDKTKRIFLKAESEEEIFAHLGLDYIEPWERNA

SEQ ID NO: 80 - protein, 379 aa, artificial sequence
  N14 trunc new  [92.08 percent identity to wt(Piliocolobus tephrosceles) calc by NCBI BLASTP]
SPSPVPGSQNVPAPVVQKISQYACQRRTTLNNYNEIFTRAFDILAENSEFRENEDSYVTFRRAASVLKSLPFTIISMKDTEGIPCLGSKVKRIIEEIIEDGESSEVKAVLNDERYQAFKLFTSVFGVGRKTSEKWFRMGFRTLEKVRSDESLKFTRMQRAGFLYYEDLVSRVTRAEAEAVSVLVKEAVWAFLPDAFVTMTGGFRLGKKMGHDVDFLITSPGSTEDEEQQLLQKVMNLWEKKGLLLYYDLVESTFEKLRLPSRTVDALDHFQKCFLIFKLPLQRVGSDQSSWQEGKTWKAIRVDLVMCPYERRAFALLGWTGSAFFNRDLRRYATHERKMILDNHALYDKTKRIFLKAESEEEIFAHLGLDYIEPWERNA

SEQ ID NO: 81 - protein, 379 aa, artificial sequence
  N100 or trunc new  [93.14 percent identity to wt(long Rattus norvegicus) calc by NCBI BLASTP]
SPSPVPGSQNVPAPVVQKISEYACQRRTTLNNHNELFTRAFDILAENYEFRENEESRDAFRRAASVLKSLSFPIVSMKDIEGIPCLGDKVKRIIEEIIEDGESSEVKAVLNDERYKAFKLFTSVFGVGRKTAEKWFRMGFRTLEKIRSDKSLRFTHMQKAGFLYYEDLVSRVNRAEAEAVSMLVKEAVVAFLPDALVTMTGGFRLGKMTGHDVDFLITSPEATEEEEQQLLHKVTNFWRQQGLLLYCDIIESTFEKFKLPSRTVDALDHFQKCFLILKLHRGLVRSEESGQQEGKDWKAIRVDLVMCPYERRAFALLGWTGSPFFNRDLRRYATHERKMMLDNHALYDKTKRVFLEAESEEEIFAHLGLDYIEPWERNA

SEQ ID NO: 82 - protein, 366 aa, artificial sequence; identical to SEQ ID NO: 68
  N100 trunc new  [89.86 percent identity to wt short Rattus norvegicus calc by NCBI BLASTP]

SEQ ID NO: 83 - protein, 383 aa, artificial sequence
  F57 trunc new (Poecilia reticulate)  [56.66 percent identity to wt calc by NCBI BLASTP]
SPSPVPGSQNVPAPSVDKISQYACQRRTTLNNYNKKFTDAFEILAENYEFNENKGFCLAFRRAASVLKFLPFTIVRMNDIQGLPWMGDQVKRVIEEILEEGESSKVKEVLNDEKYKAFKLFTSVFGVGRKTSEKWYRMGLRTLEEVKADKTLKLTKMQKAGFLYYEDLISRVSKAEADAVSLIVKDTVWKFLPDALVTLTGGFRLGKKMGHDVDFLITTPGPEEEEELLHKVIDLWKKQGLLLYYDIIESTFEKTKLPSRTVDALDHFQKCFAILKLHKQRVDNGNSNQSKEFDKEETKDWKAIRVDLVITPFEQYAYALLGWTGSAFFNRDLRRYATHEKKMMLDNHALYDKTKRIFLKAASEEEIFAHLGLDYLEPWERNA

SEQ ID NO: 84 - protein, 378 aa, artificial sequence
  F32 trunc new  [89.95 percent identity to wt Anabas testudineus calc by NCBI BLASTP]
SPSPVPGSQNVPAPSVATVSQYACQRRTTTENNNKIFTDAFEVLAESYEFNEMEESRDAFRRAASVLKSLPWAVQSLGATQDLPCLGEHTKRVMEEILQYGRSFEVEKILSDERYQTLKLFTSVFGVGPKTAEKWYRRGLRSFEDIRAEPSIHLNRMQQSGFLHYGDISRAVSKAEARALGNIIDEAVHAITPDAILALTGGFRLGKEFGHDVDFIVTTPELGKEESLLPNIIDRLKKQGILLYSDYQASTFDMSKLPSHRFEAMDHFAKCFLILRLEGSQVEGGLNSAEGDSRGWRAVRVDLVSPPMDRYAFALLGWTGSAFFNRDLRRFARMERRMLLDNHALYDKTKKEFLAATTEKDIFAHLGLEYIEPWQRNA

SEQ ID NO: 85 - protein, 377 aa, artificial sequence
  F24 trunc new  [83.55 percent identity to wt Electric eel calc by NCBI BLASTP]
SPSPVPGSQNVPAPSLPTVSQYACQRRTTLDNHNKIFTDALEVLAENYEFSDSKESRDAFRRAASVLKSLPTPLRSLQDTEGLPCLGEETKRVIEEIFEEGSSSRVEDILSDERYQTLKLFTSVFGVGPKTAEKWYRRGLRSLEQVRSDPSIHLNRMQTAGFLYYEDISKPVSKAEAKALGNIIEETASAFSPSVTITLTGGFRLGKEFGHDVDFLLTVPEPGKEDGLLPAVIDRLRSQGILLYSDFQESTFDLSKLPSRRFEAMDHFQKCFLIVKLKAGLVEGEGADPGDRRDWRAVRVDLVAPPVDRYAFALLGWSGSAFFNRDLRRFARLERGMLLDNHALYDKTTNTFLQAKTEEDIFAHLGLDYIEPWQRNA

SEQ ID NO: 86 - protein, 367 aa, artificial sequence
  F15 trunc new  [80.38 percent identity to wt Salmo truttal calc by NCBI BLASTP]
SSVATVSQYACQRRTTMENHNKIFTDAFEVLAENSEFNESKESRDAFRRAASVLKSLPSAVRSLGDTEGLPCLGEHTKRVIEEILEYGRSSKVEDILSDERYQTMKLFTSVFGVGPKTAEKWYRRGLRSLEEVRAEPSIHLNRMQRAGFLYYRDISKRVSKAEAKALGNIIEETVHAIAPDAIVTLTGGFRLGKEFGHDVDFILTTPEMGKEEGLLPRVIDRLRNQGILLYCDYQESTFDMSKLPSRTFEAMDHFQKCFLIIKLKEGLVEGEGGLQSDPGDRRGWRAVRVDLVAPPVDRYAFALLGWTGSPQFNRDLRRFARLERRMLLDNHALYDKTKKIFLPAKTEEDIFAHLGLEYIEPWQRNA

SEQ ID NO: 87 - protein, 384 aa, artificial sequence
  B63 trunc new  [85.68 percent identity to wt Notechis scutatus calc by NCBI BLASTP]
SPSPVPGSQNVPAPSVDSISQYACQRRTTLNNYNKKFTDAFEILAENYEFNENKGFCLAFRRAASVLKFLPFTIVRVNDIEGLPWMGEQVKRIIEDILEEGESSKVKAVLNNENYRAFKLFTSVFGVGRKTSEKWYRMGLRTLEEVKADKNLKLTRMQKAGFLHYEDLISRVSKAEADAASLIVKDTVWKFLPNALVTLTGGFRLGKKMGHDVDFLITVPGSRQEEEELLHTVIDLWKKQGLLLYYDLIESTFEKTKLPSRTVDALDHFQKCFAILKLHKERVDKGNSIQSKAFAEEETKDWKAIRVDLVVTPFEQYAFALLGWTGSPFFNRDLRRYATHEKKMMLDNHALYDKTKKIFLKAASEEEIFAHLGLDYLEPWERNA

SEQ ID NO: 88 - protein, 380 aa, artificial sequence
  B59 trunc new [84.74 percent identity to wt xenopus calc by NCBI BLASTP]
SPSPVPGSQNVPAPSEVKVSQYACQRRTTLQDTNRIFTDAFDILAENYEFNENKGPCVAFRRASSVLKSLPFPIVAMKDLEGLPWLGDQMKRIIEEILEEGKSSKVVEVMNDERYKAFKLFTSVFGVGRKTSEKWFRMGFRTLEEIKSDKELKLTKMQKAGLLYYEDITSAVSKAEAEAVEQLIKSIVWKFVPDAIVTLTGGFRLGKKMGHDVDILITTPRKGEKEEILHKTINVLKKRGLLLFYNIIESTFDETKLPSRHVDALDHFQKCFTILKLQKQQLDNGNSNEPFETEKKNWKAIRVDLVITPYEQYAYALLGWTGSPQFNRDLRRYATHEKRMMLDNHALYDKTKNIFLKANSEEDIFTHLGLDYLEPWERNA

SEQ ID NO: 89 - protein, 383 aa, artificial sequence; identical to SEQ ID NO: 83
  B57 trunc new [83.03 percent identity to wt alligator calc by NCBI BLASTP; 84.07 % id to purple]

SEQ ID NO: 90 - protein, 370 aa, artificial sequence
  B19 trunc new [92.35 percent identity to wt neopelma calc by NCBI BLASTP]
SSVSKVSQYSCQRKTTLNNYNKKFTDAFEIMAENYEFKENEIFCLEFRRAASVLKFLPFPVTRMKDIQGLPCMGDRVRDVIEEIIEEGESSRVKEVLNDERYKAFKQFTSVFGVGRKTSEKWYRMGLRTLEEVKADKTLKLSKMQKAGFLYYEDLAERVSKAEADAVSLIVKNTVVTFLPDALVTITGGFRLGKKIGHDIDFLITNPGPREDEELLHKVVDLWKKQGLLLYCDIIESTFVKEQLPSRKVDAMDNFQKCFAILKLYQPRVDNSSYNTSKKFDMAEVKDWKAIRVDLVITPFEQYAYALLGWTGSPQFNRDLRRYATHERKMILDNHALYDRRKRIFLKAGSEEEIFAHLGLDYVEPRERNA

SEQ ID NO: 91 - protein, 369 aa, artificial sequence
  specific bovine from Misha
KTPPLVVKKISQYACQRKTTLNNYNEIFTRAFEILAENSEFKENEESYVTFRRAASVLKSLPFTIISMKDTEGIPCLGDKVKRIIEEIIEDGESSEVKAVLNDERYQAFKLFTSVFGVGRKTSEKWFRMGFRSLEKIRSDKTLKFTKMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSAFFNRDIRRYATHERKMMLDNHALYDKTKRVFLKAESEEEIFAHLGLDYIEPRERNA

SEQ ID NO: 92 - protein, 374 aa, artificial sequence
  specific latmeria from Misha
NVPAPSVVAISQYACQRRTTLNNHNKIFTDAFEILAENYEFNENEGPCLAFRRAASLLKSLPYAISSMKDLEGLPCLGDQTKAVIEEILEEGQSSKVQDVLSDERYKSIKLFTSVFGVGLKTAEKWYRKGFRTLEEVQADKEIKLTKMQKAGFLYYEDISSAVTKAEAEAIGQIIEDTVRLFAPDAIVTLTGGFRLGKKIGHDVDFLITTPETGNENGLLHKVINVLQNQGILLYYDIVESTFDKTRLPSRKVDALDHFQKCFAILKLHKQKVNTSNSEEAEEPSNTETKDWKAIRVDLVITPFDQYAFALLGWTGSAFFNRDLRRFATHERKMMLDNHALYDKTKKIFLPAKTEEDIFAHLGLDYIEPWERNA

SEQ ID NO: 93 - protein, 370 aa, artificial sequence
  specific Puma from Misha
KTESAVVKKIPLYACQRRTTLNNFNEIFTRAFEVLAENYEFKENEISSATFRRAASVLKSLPFTIISMKDTEGIPCLGDKVKRVIEEIIEDGESSEVKAVLNDERYQAFKLFTSVFGVGLKTSEKWFRMGFRTLEKIKSDKTLKFTQMQKAGFLYYEDLVSGVTRAEAEAVGVLVKEAVWAFLPDAFVTMTGGFRLGKKIGHDVDFLITIPGSTDEEEEQLLPKVINLWQRKELLLYYDLVESTFEKLKLPSRKVDALDHFQKCFLILKLHHQRVDSGKCSQQEGKTWKAIRVDLVMCPYERRAFALLGWTGSAFFNRDLRRYATHERKMILDNHALYDKTKKIFLKAESEEEIFAHLGLDYIEPWERNA

SEQ ID NO: 94 - protein, 370 aa, artificial sequence; identical to SEQ ID NO: 93
  specific N139 from Misha

SEQ ID NO: 95 - protein, 370 aa, artificial sequence
  specific shrew from Misha
NVPAPVVQKISQYACQRRTTLNNHNHIFTDAFEILAENDEFRENEESRDAFRRAASVLKSLPFSIISMKDTEGIPCLADKVKRVIEEIIEDGESSEVKAVLNDERYKAFKLFTSVFGVGRKTAEKWFRLGFRTLEGIRNDKTLKLTHMQKAGFLYYEDLVSGVTRAEAEAVGVLVKEAVWAFLPDAIVTMTGGFRLGKKVGHDVDFLITSPEATEEQEQQLLHKVITFWEKEGLLLYCDLYESTFEKLKMPSRTVDALDHFQKCFLILKLHRESVDDGTSSQLQGKTWKAIRVDLVVCPYECRAFALLGWTGSPFFNRDLRRYATHERKMMLDNHALYDKTKRKFLSADSEEDIFAHLGLDYIEPRERNA

SEQ ID NO: 96 - protein, 381 aa, artificial sequence
  M27
NSSPSPVPGSQNVPAPVVKKISQYACQRRTTLNNYNQLFTDALDILAENDEFRENEESCLAFRRASSVLKSLPFPITSMKDTEGIPCLGDKVKSIIEGIIEDGESSEVKAVLNDERYKSFKLFTSVFGVGLKTAEKWFRMGFRTLSKIQSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSPQFNRDLRRYATHERKMMLDNHALYDKTKRVFLEAESEEEIFAHLGLDYIEPWERNA

SEQ ID NO: 97 - protein, 383 aa, artificial sequence
  B10 trunc new [91.15 percent identity to wt canary calc by NCBI BLASTP]
SPSPVPGSQNVPAPSVTKVSQYSCQRKTTLNNYNKKFTDAFEVMAENYEFKENEESRDAFRRAASLLKSLPFPVTRMKDIQGLPCMGDQVRRIIEEIIEEGESSRVKEVLNDERYKAFKQFTSVFGVGRKTSEKWYRMGLRTVEEVKADKTLKLSKMQKAGFLYYEDLVSRVSKAEADAVSLIVKNTVVTFLPDALVTITGGFRLGKKIGHDIDFLITNPGPREDEELLHKVIDLWKKQGLLLYCDIIESTFVKEQLPSRKVDAMDHFQKCFAILKLYQPRVDNSTSNTSKKLEMAEVKDWKAIRVDLVITPFEQYAYALLGWTGSAFFNRDLRRYASHERKMILDNHALYDRRKRIFLKAGSEEEIFAHLGLDYVEPWERNA

SEQ ID NO: 98 - protein, 381 aa, artificial sequence
  Mouse TdT mutant  >TH M27 (A146V, G189E, K394T)
SGSPSPVPGSQNVPAPVVKKISQYACQRRTTLNNYNQLFTDALDILAENDEFRENEESCLAFRRASSVLKSLPFPITSMKDTEGIPCLGDKVKSIIEGIIEDGESSEVKAVLNDERYKSFKLFTSVFGVGLKTAEKWFRMGFRTLSKIQSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSPQFNRDLRRYATHERKMMLDNHALYDKTKRVFLEAESEEEIFAHLGLDYIEPWERNA

SEQ ID NO: 99 - protein, 381 aa, artificial sequence
  Mouse TdT mutant (MS 13-34 (A146V, G186E, K394T, Q455F, W506R))
SGSPSPVPGSQNVPAPVVKKISQYACQRRTTLNNYNELFTRALDILAENDEFRENEESRDAFRRASSVLKSLPFPITSMKDTEGIPCLGDKVKRIIEEIIEDGESSEVKAVLNDERYQAFKLFTSVFGVGRKTAEKWFRMGFRTLEKIRSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSAFFNRDLRRYATHERKMMLDNHALYDKTKRVFLEAESEEEIFAHLGLDYIEPRERNA

SEQ ID NO: 100 - protein, 381 aa, artificial sequence
  Mouse TdT mutant (MS 34-1 (A146V, G186E, K394T, Q455F))
SGSPSPVPGSQNVPAPVVKKISQYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSAFFNRDLRRYATHERKMMLDNHALYDKTKRVFLEAESEEEIFAHLGLDYIEPWERNA

SEQ ID NO: 101 - protein, 381 aa, artificial sequence
  Mouse TdT mutant (TH c2_5 (A146V, G189E))
SGSPSPVPGSQNVPAPVVKKISQYACQRRTTLNNYNELFTRALDILAENDEFRENEESCLAFRRASSVLKSLPFPITSMKDTEGIPCLGDKVKRIIEEIIEDGESSEVKAVLNDERYKSFKLFTSVFGVGLKTAEKWFRMGFRTLEKIRSDKSLRFTQMQKAGFLYYEDLVSGVNRPEAEAVSTLVKEAVVTFLPDALVTMTGGFRLGHMTGHDVDFLITSPEATEDEEQQLLHKVTDFWKQQGLLLYCDILESTFEKFKQPSRKVDALDHFQKCFLILKLDHLRVHSAKSGQQEGKGWKAIRVDLVMCPYDRRAFALLGWTGSVQFKRDLRRYATHERKMMLDEHALYDKTKRVFLEAESEEEIFAHLGLDYIEPWERNA

SEQ ID NO: 102 - protein, 361 aa, artificial sequence
  (M57 without His tag & linker & with Q4E mutation) bovine-mouse chimera
KISQYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 103 - protein, 361 aa, artificial sequence
  (M59 without His Tag and linker & with Q4E mutation) bovine-mouse chimera
KISQYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLAERVNRPEAEAIRMLVKEAVVTFLPDALVTMTGGFRLGKMTGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 104 - protein, 362 aa, artificial sequence
  (M27 with maximal N-terminal deletion plus Q4E mutation) mouse
KISEYACQRRTTLNNYNQLFTDALDILAENDEFRENEESCLAFRRASSVLKSLPFPITSMKDTEGIPCLGDKVKSIIEGIIEDGESSEVKAVLNDERYKSFKLFTSVFGVGLKTAEKWFRMGFRTLSKIQSDKSLRFTQMQKAGFLYYEDLVSGVNRPEAEAVSMLVKEAVVTFLPDALVTMTGGFRLGKMTGHDVDFLITSPEATEDEEQQLLHKVTDFWKQQGLLLYCDILESTFEKFKQPSRTVDALDHFQKCFLILKLDHPRVHSVKSGQQEGKGWKAIRVDLVMCPYDRRAFALLGWTGSPQFNRDLRRYATHERKMMLDNHALYDKTKRVFLEAESEEEIFAHLGLDYIEPWERNA

SEQ ID NO: 105 - protein, 361 aa, artificial sequence
  M54 with maximal N-terminal deletion plus Q4E mutation
KISEYACQRKTTLNNYNEIFTRAFEILAENSEFKENEESYVTFRRAASVLKSLPFTIISMKDTEGIPCLGDKVKRIIEEIIEDGESSEVKAVLNDERYQAFKLFTSVFGVGRKTSEKWFRMGFRSLEKIRSDKTLKFTKMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSAFFNRDIRRYATHERKMMLDNHALYDKTKRVFLKAESEEEIFAHLGLDYIEPRERNA

SEQ ID NO: 106 - protein, 362 aa, artificial sequence
  M77 without His tag
KKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 107 - protein, 361 aa, artificial sequence
  C1-40 without His tag
KISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 108 - protein, 363 aa, artificial sequence
  A2-20 without His tag
VTTVSEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 109 - protein, 363 aa, artificial sequence
  ii-ell-40 without His tag
VATVSEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEKIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSPQFNRDLRRYATHERKMMLDNHALYDKTKRVFLEAESEEEIFAHLGLDYIEPWERNA

SEQ ID NO: 110 - protein, 363 aa, artificial sequence; identical to SEQ ID NO: 109
  9-40 without His tag

SEQ ID NO: 111 - protein, 365 aa, artificial sequence
  (45-751)(internal His tag)
MAEKKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTLEKFKLPSRQVDTLDHFQKCFLILKLHRQRVDSSKHHHHHHKTWKAIRVDLVMCPYENRAFALLGWTGSPQFNRDLRRYATHERKMMLDNHALYDKTKRVFLEAESEEEIFAHLGLDYIEPWERNA

SEQ ID NO: 112 - protein, 369 aa, artificial sequence
  (46-737) (internal His tag)
MAHHGSEKKISEYACQRRTTLNNYNELFTRALDILAENDEFRENEESRDAFRRASSVLKSLPFPITSMKDTEGIPCLGDKVKRIIEEIIEDGESSEVKAVLNDERYKAFKLFTSVFGVGRKTAEKWFRMGFRTLEEIRSDKSLRFTQMQKAGFLYYEDLVSGVTRAEAEAVGVLVKEAVWAFLPDAFVTMTGGFRLGKKIGHDVDFLITSPGSAEDEEQLLPKVINLWEKKGLLLYYDLVESTLEKFKRPSRQVDTLDHFQKCFLILKLHRQRVDSSKHHHHHHETWKAIRVDLVMCPYENRAFALLGWTGSPQFNRDLRRYATHERKMMLDNHALYDKTKRVFLEAESEEEIFAHLGLDYIEPWERNA

SEQ ID NO: 113 - protein, 552 aa, Saccharomyces cerevisiae
MASQKVFGITGPVSTVGATAAENKLNDSLIQELKKEGSFETEQETANRVQVLKILQELAQRFVYEVSKKKNMSDGMARDAGGKIFTYGSYRLGVHGPGSDIDTLVVVPKHVTREDFFTVFDSLLRERKELDEIAPVPDAFVPIIKIKFSGISIDLICARLDQPQVPLSLTLSDKNLLRNLDEKDLRALNGTRVTDEILELVPKPNVFRIALRAIKLWAQRRAVYANIFGFPGGVAWAMLVARICQLYPNACSAVILNRFFIILSEWNWPQPVILKPIEDGPLQVRVWNPKIYAQDRSHRMPVITPAYPSMCATHNITESTKKVILQEFVRGVQITNDIFSNKKSWANLFEKNDFFFRYKFYLEITAYTRGSDEQHLKWSGLVESKVRLLVMKLEVLAGIKIAHPFTKPFESSYCCPTEDDYEMIQDKYGSHKTETALNALKLVTDENKEEESIKDAPKAYLSTMYIGLDFNIENKKEKVDIHIPCTEFVNLCRSFNEDYGDHKVFNLALRFVKGYDLPDEVFDENEKRPSKKSKRKNLSSGENLYFQGSSGS

SEQ ID NO: 114 - protein, 674 aa, Unknown
  Myceliophthora sp.
MAERIYGVTPPISTALPTEEEKRLNNALHQELRAQGTFESPAETEKRKEVLRQLEKITNVFVQRAAAEKEPKNTILIRDAIGRVFTYGSYRLGVYGPGSDMDTLVVAPKYVTVEQYFRIFPEVLVEMAPPGAITDLTPVPEAFVPIIKFEFSGISIDLIFCSIQTLKQLPADKNWSLADNNLLRGLSENEVRSLNGTRVTDDILNLVPEPATFKLALRAIKLWAQRKAIYANIMGYPGGVAWAMLVARVCQLYPKATSAVIVNKFFHIMLKWPWPLPVLLKDIEYGCPVTRVAVWNPKIYASDRNHRMPIITPSYPSMCATHNVGRSSMAVIKDELEKGVQVTEDIMRGKRPWKDLFTKHTFFTSGFRYYLTVISSSRTKKAQNVWSGFVESRVRLLVNKLEMHPSISLARPFNKGYDREHRCKNNAQLEEVVSLGSLAYMYKPATGEAKAEAKKEVKTEVKHESGASVKNEAPEAKGEDGVRAKRENGDEAQLPPATGIKPEPTDGAGADVKLEDIPVKKDEPEEMTIYTTNHYIGLQLVEGAKTLDLSREVNDWKAMCTSNELYEEGTMFLSIQHVRNTALPDDVFEPGETRPRPAKKSLKRVASEDPGNKQTQPPAKKQVQDKAPPAAQQQQQQQHQQQQQPASTAAAAAGSSGENLYFQGSSGSHHHHHH

SEQ ID NO: 115 - protein, 672 aa, Unknown
  Thielavia sp.
MAERTYGVTPPISTALPTEQEKALNKALHDELRAQGTFESRAETEKRKEVLAQLEKITNAFVQRAAREKHGKNAILIRDAIGRVFTYGSYRLGVYGPGSDMDTLVVAPKYVTVEQYFRIFPEVLVEMAPPAAITDLTPVPEAFVPIIKFEFSGISIDLIFCSIQTLIQLPADKSWSLADNNLLRGLSENAVRSLNGTRVTDEILHLVPEPATFKLALRAIKLWAQRKAIYANIMGYPGGVAWAMLVARVCQLYPKATSAVIVNKFFNIMLKWPWPLPVLLKDIEYNGPVTRVPVWNPKLYASDRNHKMPIITPAYPSMCATHNVGRSSMVVIQQELKKGAEVTEEIMLGRRPWKDLFTKHTFFTSGFKYYLTVISSSRTKKAQNVWSGFIESRVRLLVNKIEMHPSIALARPFNKGYDRMHRCKNDAQVEEVVSAGSLAYVYTPPAFGDEKVKSETKSEVKQEVKQEVRQDDVIQDGVPVKQEKAEVRAEDGVRIKRELSEEVQLPPPTNVKPEPHDDEAKVKLEDIPEKEAQPEDMEIYTTNHYIGLQLVEGAKSLDLSREVNDWKAMCMSNELYEEGLMFLSIQHLKNTALPDDVFEPGEVKPRPGKKVLKRGASEEPSKQQPPAKRQAHVQPRAPAAQQPSSTATAAAGSSGENLYFQGSSGSHHHHHH

SEQ ID NO: 116 - protein, 566 aa, Pyronema omphalodes
MSAPPKQYGTTPPINTNPPTKEDMERNSELVQELKNQNCFEEKAESDKRVKVLEILQTLGEAFVKKACAAKGLPDHLVNNSGGKIFTFGSYRLGAYGPGSDIDTLLVVPAHIERADFFQHVPKMLSELNPPAEEVSPVPDAFVPIIKFELQSISIDLIFAKVPSVNSIPRDMTLENKDILKGCDEANLRGLNGVRLTDELLGLVPHPVNFRMALRAIKLWAKSRAIYANVMGFPGGIAWAMLVAKICQLYPMAVSAVIVCKFFTIYTKWKWPQPVLLKDILDEGGQGAGLRVWNPKIYASDRGHLMPVITPNYPCMCSTHNITKSTKAVILREMDRSLNIIHEIMDGKSPWSKLFAPHTFFTQDYRYYLRVISAARTKDEQLLWSGLVESKLRHLVSKLEMLDNIQLAHPFNKGFEYEVECNNEDEVMRVIRGENIDQSEANGTAVDSNGKSSKVKAYTTSFYVGLVLDTSKSKQFDISWACHEFYDICKTWNLYNDDMHSIHVVNTRNFELPDNVFKPGETKPTKKTKTIKRKIPNAAGKKRGADEISAPDSKRAHINEVPPNAN

SEQ ID NO: 117 - protein, 598 aa, Tilletia indica
MTKHLGVTPIVSDASPTAHEIALETQLVTELTTQNCFESPQDSRLREVVLGKADKLVKEFVYRAAIARGLPESAARELGGKIFTFGSYRLGVHGPGSDIDTLCVVPKHVQREDFFTIFEQMLRQREDVTDVAPVPEAYVPLIAVNFMGIAIDFLFARLSLSRIDDSLDLSDNTILKNMDDQDIRSVGGSRVTDEILRLVPNVETFHTALRAIKLWAQRRAIYKNMVGFPGGVAWAMLVARICQLYPNGNAAVIIERFFIIMFQWNWPQPVMLKHHIYEADPILKVWNPRLYAADRSHRMPIITPAYPGMCATHNITASTQAVLTLEFKRGIEIMSAMSASITNPALPQTTWATLFESRRFFEEYKYYLQIIASSGSADLQLKWAGTVESKLRHLVLSTEAQPGVKIAHPYTREIDLVSECHTDEEVRRVATGDVPPEVAARARTVEGEGVLPESKEVDKLKEIESKEEEARKKGRRTIWTTTFYIGLALHDGSDDKTGQSAPKRLDLVGPIQDFKGRCTEWATYDESTMGVVVRHVKRSQLPDSVFPDGKRPAAPVKKVVKKRKRNAGMAEAAAETKEEEGADEQEAKRARTNGDSAA

SEQ ID NO: 118 - protein, 574 aa, Clathrospora elynae
MNGPPKTQYGVTSAISEAAPTEEDRLLNDKLIETLKRENVFETPEGNAKREEVILHLQKVVEEFVRRVGKQKGVPQSTIDAAGGKVFTFGSYALGVHGPSSDIDTLVVAPKFVTIDEFFQTFPPTFKEMSRVEDIKEFVPVEDAFVPIIKMEYRGVSIDLLFASLPRMASIPKDMDTIDKKNLEGLGESATRSVNGTRVTKELLAAVPQNNSFRHALRAIKLWSSRRGIYGAVFGYPGGVAWAIMVARICQLYPFANGATIVSKFFSLMYKWTWPRPVMLKHIEEGAMGLRVWNPQIYGGDRAHLMPIITPAFPSMCATHTVMPSTLRIMKEEFGRADKILQHVFAGTKKWDALFERHSFFTKDHKYYLSVVAASRTKEANSTFSGLVQSKIRHIVKGIDDGQTGVDIARPYIDYFERYHRCKDEDQIFQVCQGKLDHMIPASELPAEGTAPANGDSHIMYTTTFYIGLTLPLGTATEGTKSLDISYPVSQFKNFITDADKFDKETMSVKVVHTRSSALPDDVFVQGETRPKKPVKEKKKKEAKSAKRQFADTGLDVRDSRLTKPSQRSSASRE

SEQ ID NO: 119 - protein, 546 aa, Drechslerella brochopaga
MPASVDVLKRLQKITEEFVRQVYTSKNQNELTTNSAGGKVFTYGSYRLGVVGPGSDIDTLVVAPKLVTREDFFKFYPPLLKALNKANEPPVIEELAPVPDAFVPIIKFVMSGISIDLIFCRLGVAQVPADMTLEDKNLLRGLDEKELRSLNGTRVTDEILTLVPVPAVFKHALRAIKIWAKCRAIYGNVYGFPGGVAWAMLVARICQLYPSAVSAVIVSKFFRILGQWNWPQPVLLKPIEDGPLNVRVWNPKLYPSDRNHLMPIITPAYPSMCSTHNITPSTKAVILGEMAKAADIVDRIITGNGNWNQLFQRHTFFTKDYKYYLTVKASARDQEQSVKWSGLVESKIRHLVMKLEMLSDVIASARPYVKPFEKVQYCRNQEEAHKIAMGQHVPDSPPAEAANAATGETAGEVSEQEKEGGVPVWITTFHIGIELTPGMVGQVNISWPAQEFYIMCHNWEGYEENMHCVKLELLRNWNLPDECFDFSKGDAKPSRPIVKRKVIRKVVSSSEVSQSGMKRTHAEENPNADEGDVKRTKVNAPVTVQG

SEQ ID NO: 120 - protein, 632 aa, Magnaporthiopsis poae
MAERQLGVTPPVSVSLPSEFELQATDALLAELRAQNSFESPLETQKRHRVLASLQTIADAFVKVVAQEREPHNAVLIKDARAKVFAYGSLRLGVWGPGSDIDTLVVGPRYVTREDYFKHFPDLLVKMAPPGAITDLAVVQEAFVPIIKFEYHGISIDLIFSRIATLKQLPSDPAWDLKDNNLLRGLDEKELRSVNGTRVTDEILSLVPEQNIFRTALRAIKLWAQRRAVYGNIMGFPGGVAWAMLVARVCQLYPKATSSVIVNKFYSILLQWPWPQPVLLKPIGDGPLQVRVWNPRLYKGDSYHLMPVITPAYPSMCATFNVTRSSMTVIYRELQLAAEVTNNIMCSTKPWKDLFTKHTFFTKDFKYYMQVISASRDKEAHKIWSGFVESKVRMLVQSLERHDSIAVARPFIKGFERFHRYRNEEQFAQILDGNLQHMVKAGGDASPSQDGIQAKKEDNSSVKAEEDAKVKQENGETPVKQENGVKVKGEESEEAPLASIPEHSPVDTKGDSKAGDEALEMYTTTHYIGLELNPAAKSLDLSFQVNDFRALCHNWEKYKDELSDRCYVNIKDLRNFALPEDLFEPGEVKPVKSVKKRPAAGANATGDKKRPANEDNSNPAKRRQAASVAAAG

SEQ ID NO: 121 - protein, 631 aa, Cryptococcus depauperatus
MLGVTPPISIEAPKPGDIHSSEALMTDLIAMNQFESDSERKIREKLLSNIAQLVVKFVHDVSLKIGMSEKMASESGGRIYTSGSYRLGVHGPGSDIDTICVCPRHIYREHFFGEFQEMLRAWPEVTEITAVTGAFVPVMKTVISGVEVDLLFARVNLPEAGDSLDIEKDEILRGVDDASQRSLNGPRVTDMILNLVPDVTTFRTALRSIRLWARRRGIYSNVLGFPGGVAWALLTARICQLYPNATPSIIVGKFFPIYYQWNWPQPVILKKIENGPPNMQHAVWNPKLDRRDMAHRMPVITPAYPSMCSTHNITASTMSIISKEMLRAMQITDDILREPGASWAPLFEKVDFFSMYKTYVQVVASASTAEGIKDWSGTVEARIRTLVQDLENTDSILTAHPLVGGVSRVFYCTTEEEQAAASQGELTSEMIDRTEEEVTDKEHRNIFTKSFFIGLEIEKKTKEGGGRVLNLFYPSKKFCSMCQSWDRYNEMEMSVILRPAKRSDLPSFVFPDGIPISKKKAKRQQQNGSADASLSDSVEGQGPSKRTKAAPPSPNQASNPRCNGLPVQNGSDPVSSKIGEEVDIKPPPGIENLPPLSNAAMSSFATAAKGVATTQDFNKEGLVVLNQQSTL

SEQ ID NO: 122 - protein, 571 aa, Golovinomyces cichoracearum
MAEKSFGVTAPLSVTPPTESENQASSALIEELKRQNNYENAIETANRQKVLNSLQLITEEFVRQVSRAQGLPANLIKSAGGMVVTFGSYKLGVIGPGSDIDTLIVAPQNVTKEDFFSRFPDLLRSMATEGNITELTAKPDAFAPCITLKYAGIDIDLLFGRVKLSQVPRNLSLLNQNMLRGLNNEEVRSLNGVRVADEILNLVPEPAIFRTALRTIKLWGTRRAIAGNIYGFPGGVTWAILVARICQLYPKATSSTIVFKFFRIMEKWRWPMPVLLKEIDNLNALGLKVWNPKIYGSDKNHIMPIITPAYPEMCTTHNFSLSTKAILEKELKRGGDITDRVMSGKASWKDLFAKHTFFTNGYKYYLSVVSASRNKESQLFWSGFVESKVRLLVNKLEYHPSIALAHPFNKGFTRVHRCRTEEEVDLVKNGSLKFHATDIATSTTGHGLSVETTSKEKLNETKNVEQDENLIMVYTATHYIGLELAKGAKSLDLSYEVEEFKSICTSSEAYNQEENSLGVAHTKNCDLPDDVFTEGELKPTRPLKQKKKRPATEDSNPVPSKRQQTSLIAVG

SEQ ID NO: 123 - protein, 600 aa, Hortaea werneckii
MAEKQYGVTPAFSLEPPSPKDLKLNDALLAEFKAQNNFAPQSDTEKREAILKKLEGLLQRMVQEVGRKKGLPQSILEVAGGKVFTYGSYRLGVYGPNSDVDTLMVGPKHVTREDFFEHMPPLIRSAWAEDQIGGLVPVPGIGTPIIKLELEGVDIDLIYSSLQLSSIPKDIELKDDNLLRGLDDTDRRCVNGTRVTNRILELVPQTKTFRLALRAIKLWSSQRAIYGNIVGFPGGVAWAILVARVCQLYPKAAAPLLISKFFFIMKRWNWPKPVFLQHKEETSLQLREWDPIQYRGDGFHLMPILTPAYPSMNTAHTVGPSTKMIIMRELERGENIVNDIYANKRPWKDLFQRHTFFTNAYKHYICVVVAAKNKDAHDNWSGLVNSKLKFLVKGIEDSGGSSVELVQPFNKGFSRVHECQTNEQVEKVLDGSLECQVKETKTTEEGNDTAIANGVAQTDTEGLEVPKADSEARAEGSGTTKLWTTTFYLGIGLKKGATDLDISVPVRNLQGDCTAWADYNPDLHSIKIKHIRNFNLPDDVFKEGEVKPQRPKKKTNAAKAAEATSNKRSFSDTGLDPNADPAKRRQSGNVPTPVANGSVG

SEQ ID NO: 124 - protein, 589 aa, Valsa sordida
MAEIQYGMTPPLSTELPKDPEKRANDALFAELKAQNTYEHATETQKREKVLKSLQSIADVFVQKVAQRVHKESPAIQKSARGRVFTYGSYRLGVFGPGSDIDTLVVAPKYVTREDFFELFPGLLKELAPAGSITDLTAVTDAFVPIIKFEYSDISIDLIFSRIATLTEIPALTTKWDLLDNNLLRGLDDAELRSLNGTRVTDDILKLVPEQTSFRLALRAIKLWAQRRAIYANIMGFPGGVAWAMLVARICQLYPKANGAVIVDKFFHIIRRWPWPQPVLLKNPEDGPLQVRVWNPKVYKGDSYHLMPVITPAYPSMCSTFNVTHSNKAVIQKELDHFADVVQQIMMGKLPWKSLFVKHTFFTKDYKYYIAVIAASTTKENSKIWGGFVESKIRLLVQGLERHSSIRLARPFNKGYERKHRTVSGMNEWHDILDGSLDYVVKDEQVADEKAKTEPGNDAVKTEPTSPAKVKQEEGIESLEIKDDAKETDGKNEQDEEDAEVKPETEDPDSPKDVYTSTHYIGIELHDEHYSWSLPDDVFEAGETKPTRPLKKKAAQPPNGAKRKTPTEGALNSAIDAAKRQKTSTVSAG

SEQ ID NO: 125 - protein, 588 aa, Wallemia mellicola
MAEKQWGITPPISLASPTDLEKQVDAGLIQELKDQNTIESEQESLNREKVLALVQSLVKEFVKRVSIRNGMSESLADQAGGKIFTFGSYRLGVNQPGADIDTLCVVPKHVSREDFFDVFEPLLKTREEVSVCAGVPDAYVPIIKTTISGIEIDFLVARLALATIPDDLELADDNLLKNLDERCVRSLNGSRVTDEILRVVPNVQVFRESLRCIKLWAQRRAIYSNVNGFLGGVAWAMLVARICQLYPNENAGAVIAKFFTILYQWKWPHPVLLKEIEDGPLKVRVWNPKVGSSTLLIYPSDRAHRMPIITPAYPSMCSTHNVGPSTQEVMRQEFKRGMDILDGIHKGTTTWSQLFNKHEFFSKYKHYLQIIASGQTAESQKKWSGAVESKVRQLVSKLELVDGIELAHPFVKGFSEVFICLNDEEVQHAAEYNPSEEVKERSKKPEEYENKEAEDGIHRVYISTFYIGLAIQPRNPETNEKRTLNLTYPTNEFMKLTKLWDQFVEGEMKIFVKNIKASALPDVVFEGGIRTSSSKSSKKKSKRPSNVMDESNGENEQNKRLKPDNTSNNEQKSEATALAPSAVPTEST

SEQ ID NO: 126 - protein, 587 aa, Xylaria flabelliformis
MAEKTWGITKPISSAPPTPAESHATALLLEELRRQNTFETASEIKKREKVVNDLQRIADEFVRKVARAKEPHNEVLIRDARGEVFTYGSYCLGVYGPGSDIDTLVTAPRYVTRDDYFKYFPDLLTEMAPPGAITNLTAVEDAFVPIIKFEYWGISIDLIFSRIATLTQFPPHKQLELTSNEHLRGLDDRELRSLNGTRVTKEILNLVPEQSTFRTALRAIKLWAQRRAIYANIIGFPGGVVWAMMVARVCQLYPKATSATIVGKFFLVMKGWPWPIPVQLKHMEDGPLNVRVWNPKIYKSDSFHLMPVITPAYPQMCATFNITKSTKTIIQRELERGVELTDKILGSERPWKDLFVKHTFFTQDHKYYLAVIATSTTKEAHKIWSGFVESKVRILVGELERHSSIALARPFNKGFERQHLTKTDQQAAEVQSGSLAYVTEDEDNSDDATVTKAENGTDGNADSSDQKVTAENTTQPRRTYTTTHYIGLELAEGSKSLDLSYQVNAFKQLCSEWEKYSAEFNSLSVQHVKNINLPEDVFEPGETKPARPLKKPTANGTAPTTQRKRNAPTEGHPPPAKRQQSSAAAAG

SEQ ID NO: 127 - protein, 592 aa, Chaetomium globosum
MSEIIYGVTPPISTTLPTEPEKRLNHALHQELRAQGTFESPQETEKRKEVLRQLEKITTVFVQRAAAQKEPKNTFMIRDAIGRVFTYGSYRLGVYGPGSDMDTLVVAPKYVTVEQYFEIFPEVLVEMAPPGAITDLTPVPEAFVPIIKFEFSGISIDLIFCSIQTLKQLPDEKNWSLADNNLLRGLSENEVRSLNGTRVTDDILHLVPEPATFKLALRAIKLWAQRKAIYANIMGYPGGVAWAMLVARVCQLYPKATSAVVVNKFFHIMFKWPWPLPVLLKDIEYGSPVTRVPVWNPKSSRRAGPKRHRTSMRASSNRGCGCWLTDLRGTILLPWPGPSTRVTTENTDARTIGSWEEVVSVGSLAYVYKPPADSEEKAKAEPKVEPKREVKQEIKAENREPAAAEEKLETRGEDGMRIKQEHSESTQPPPPSNVKPEPHHDTNSEVKLEDIPQKKKDEPDEMEIYTTNHYIGLQLVEGAKSLDLSREVNDWKSMCTSNELFDEGLMFLSIQHLKNTNLPDDVFEPGETKPRPVKKNLKRAASEGPGNMQPQPPVKRHAPGKPSSHSAQQQQQQQQKQRQQRPASTAAAAVAG

SEQ ID NO: 128 - protein, 591 aa, Lachancea thermotolerans
MNQQKTYGVTGPISTAGPTAAENQLNDALIQELKKEKSFESEEDTKKRVEVLRILQNLAQEFVYQVSKKRNMSDGMAKDAGGKIFTYGSYRLGVHGPGSDIDTLVVVPKHVTREDFFTVFDQILRTRSELEEIAPVPDAFVPIIKIKFSGISIDLICARLDIAQVPVNLTLADKNLLRNLDEKDLRALNGTRVTDEILQLVPKPTSFKIALRAIKLWAQRRAVYANIFGFPGGVAWAMLVARICQLYPNACSAVILTRFFHILTKWNWPQPVLLKPIEDGPLQVRVWNPRIYAQDRSHKMPVITPAYPSMCATHNISESTKKVILAELERGAQISSEIFSNKKTWSDLFQKHDFFYKYKFYLTVMASTSGSSEQHLKWSGLVESKLRLLVQKLETLGGINLAHPFTKPFEASYVYDNEAQCKDIIDNYGTHKAQDILAQYTEVTDDNKDTDGVKDKAQVHITTMYIGLDVALDGKKQVDIHVPCSDFFNLCRSFSEYDDTDMFSLMIKYVKLYDLPDNVYVEGEERPVKHSKRKKLGKDSKKSKRPKSNSLKAEEPTSGTTNSEKNNSQGPNKPVKHEPPMQTSPNPLAGS

SEQ ID NO: 129 - protein, 558 aa, Schizosaccharomyces octosporus
MSVKQWGITPPISTAPATEQENALNTALIDELKRQNLFESSAESERRIKVLDDLQRIATEFVKKVSLAKHMNEKMANEAGGKIFTYGSYRLGVYGPGSDIDTLLVVPKHVSRENFFQDLEPMLREREEITELASVPDAYVPIIKFKYFDISIDLIFARLSVPKVPRTLELSDNNLLKGVEERCILSLNGTRVTDQILQLVPNRAVFKHALRAVKFWAQRRAIYANVIGFPGGVAWAMMVARICQLYPNAVSSVIVAKFFRILHQWNWPQPILLKPIEDGPLQVRIWNPKLYPSDKAHRMPIITPAYPSMCATHNITPSTQAIILSEMVRAGEIADQIMVGSVGWSALFQKHDFFHRYKHYLMITAASKTAEGQLKWSGLVESKLRHLVTRLELVEAIALAHPFNKGFDKIHNCKSEEEAHQLASGISLDVASQTADLERLAADANGQNENEDEKEEKKIYPVYTTTFYIGLELEKKKGQPIKKLDISWPAQEFYELCKKWDRYDETQVNVFIKNTRNVNLPDEVFEPGEEKPKSSKKRSYNDAGNNNETMKRQKISTA

SEQ ID NO: 130 - protein, 608 aa, Exophiala spinifera
MAEKQWGMTPPMSTALPEPIDTEKTADLIEELKKENNYEPLEATQKRMATLGLLNRVTQEFVREVSRRRRMPPSQIEQFGGKIFPYGSYRLGVFGPGSDIDTLAVAPRHVTREDFFEYFPTVLKRMTAEGDISSLTPVPDSYVPIIKLVLNDIEIDLIFASIASLQTIPKNLTLNDNNLLTGLDQATIRAVTGPRVTDEILSLVPEQKTFRTALRAIKLWAQRRAVYANIVGYPGGVAWAMLVARVCQLYPHAVGATLVDKFFFVMKNWDWPTPVMLKDIEQPKPSQANEFKVWNPALYKGDKKMLMPIITPAFPSMSATYNISKSGKTVILRELERASQITNNIFAGKARWSDLFKKHSFFTADHKYYLGVTASTLNADSAKQWAGLVESKVRIFVMLLEGIPDITLARPFTKGFKRVHKCADETQIREVQKGSMKYKFEETKTVETTDPELVTSNGDGAAVPAANGAKPETDQGAHTVYTYTFYIGIDTTAKGSLNIAPSFQNFKDICEGWASFNRDSHFLTLASVKCWDLPDDVFDAKAGEVKPSRPVKKVAKQVKAEAKSGVRRSINEVEATETNGDAAKRQKLMTPTPTPTPTPAPKPTAAPA

SEQ ID NO: 131 - protein, 626 aa, Scedosporium apiospermum
MADRQLGVTPPISMALPTPAEIEANNAMVEELRKQGIFESKEETDKRNLVLESLQKMCDEFVTRVAQEKDQGLARDARGKIFTYGGFRLGVFGPGSDIDTLVVAPKYVTRDDYFAIFPDLLLEMAPKGAITGLAVVTDAFVPIIKFEYWGISIDMIFSRIAMLKKLPTNTSQFNLTDTALLRGLDETEIRSLNGTRVAHEILDLVPEQSTFQMALRAIKLWAQRRAIYANVMGYPGGVAWAMLVARVCQLYPRATAATIVSKFFCIMRQWPWPQPVLLKHIERAPLGYRIWNPVVYPSDKHHLMPIITPAYPSMNAAYNINRSSMSIIQTELSRADGITDGIVVGQRPWSDLFEKHTFFTADYKYYLAIVSSGITKDAHKKWSGFVESKVRMLVLALDRHDTIALAQAFVKGYDRVHKCNADSEIQKVQQGDLAYMIKEEDAPKEEQTSPVKSETKPDPGANEKNSAGGSNSNGDVAGSADAVKNEEGANGVKAGDENVYIYTTTHYIGLALRPGQKSINLANEVGEFKKLCKGWEKFDHQLNAITVQHLRNSDLPDDVFAEGEVKPRKRVVRKANGTGPSPPVDAKQATNGTTSQVDSKKRAAAETGAAAPAAKRRQPASVVAAG

SEQ ID NO: 132 - protein, 613 aa, Trichoderma citrinoviride
MAEQNYGVTPPISVTLPTEAENRASDALLEELRRQKTFESPSDTEKRHEICNEFVRKVAREREPKNEILIKNARGKVFTYGSFRLGVFGPGSDIDTLIVAPKYVTREDYFNYFPDLLVSMAPPGAITDLTVVKDAFVPIIKFEYSGISIDLIFSRIIQKQIAPDFHSLKDSSLLRGLDEAELRSLNGTRVTDEILELVPEKSTFKLALRAIKLWAQRRAVYANIMGFPGGVAWAMLVARVCQLYPKATMSVIVNKFFLVIGQWRWPQPVLLKPIEGGPLPVRVWNPKVYKGDSFHLMPVITPAYPSMCATFNITRSSMTIIQRELRRGLEISEQIMVGKRPWSDLFVKHTFFTSGYRYYISVVSASKDKEAHKVWSGYVESKIRMLVQKLEQHPSIALAHAFNKGYDRRHLCRNEQEIGQVQEGSLEFMIKDWDDNKLNGAAIKPEKVEGDSKPNGADIIQPEKVEGESKLSDIPVKPEMDEAESKLSEVAIKTETKDGSTESVFPVEVYTTTHYIGLELEEGAKSLDLSYQVDEFKVLCTSWKKYQEDLEHLVSLGVQHVRNFNLPDDVFEPGEQKPQKKSSAKSLANKKRGAPEDNTPPAKRQQASVAAAG

SEQ ID NO: 133 - protein, 598 aa, Aspergillus thermomutatus
MSAPPVRQWGVTPPISTALPTPDELAANDDLISELKAQNNFESPAETERRKQVLQLIQRVTHEFVKVVSRKKGLSPAAVEAAGGKIFTYGSYRLGVYGPGSDIDTLVVGPKHVLIDDFFSDFPPVLEKMAPPGAIEKMTPVPDAFVPIIKLELSGISIDLIFARLIVSSVPLNLDLKNNDYLRGLDEKEVRSLNGTRVTDEILELVPQQKTFRLALRAIKLWAQRRAIYSNIVGFPGGVAWAMLVARVCQLYPHATGSVIVGKFFRIMNKWAWPQPVLLKPIEDGPLQIKVWNPKIYHGDRFHLMPIITPAYPSMCATHNISMSTKAVILRELQRGGDIVDKIFLKQLTWNDLFARHSFFTHDYKYYLSITASSRTKEAESVWSGLVESKIRHLVGALDRKPTIAVAHPFPKGFERVHIISNEEEAEAVKNGSTKYQDKGTKTETTDETKDAAHQAAAQSGVENAEVEPVGENANGDSRIIYTTTYYIGLELKPLEPGASRSLDISTDAQIFKSTCTSWAGYQPGINDLSITHVRNFDLPEDVFQPGEARPTRPKKKVIKKPEAGAQKRGIDSLDDASLPAAKRQVTSNGISSTPTPA

SEQ ID NO: 134 - protein, 579 aa, Sodiomyces alkalinus
MQDRPLGVTPPISTSLPTDAEHAVNESLLEELKRQNTFESPAETAKREKVLEQLQLICNEFVRRVAREREAGSEALIKEARGRIFTYGSFRLGVYGPGSDIDALVVAPKYVTRDDYFKLFPGLLQEIPPTGAITDLAVVSDAFVPIIKFDFLGISIDLIFSRIASLKQLPKDKDWNLKDSNILRGLDEAELRSLNGTRVTDEIISLVPEPSTFRLALRAIKLWAQRRAVYANIMGFPGGVAWAMLVARVCQLYPKATSSVVVNKFFHIMRRWPWPQPVLLKAVESGPLQVRVWNPKLYKGDQFHLMPIITPAYPSMCATYNITKSAMTVIQRELQRGCEITDSVMMSKQPWSDLFVKHAFFTSDYKHYISVITTSTTKEAHKIWSGYVESKVRVLVQGLEQHPSIALAHAFNKGYDRRHKCSSEQEINQVQEGSLDYLLKAGDSSAGDPAEGNKSPRRPADGEHPEITPEATETIVFTTTHYIGLELVEGAKSLDLSYQVDSFKQLCGQWEKFNSNLNYLSVQHVRNVKLPDDVFEPCETRPQKKSATNGSLQKKKRGASEVDMHPPAKRQQSSVTAAG

SEQ ID NO: 135 - protein, 606 aa, Neohortaea acidophila
MDPKLGVSGTMSMEPPSPKDIKLDDALLQELKSRNEFEAPEETQRRHSVLDRLESVLKRLVAMVGKQQGLPPGILNEAGGKIFTFGSFELGVYGPKSDMDTLMAAPKHVSREDFFQYMPDLLRKEFKPEEIAELTPVPGISVPIIKLELCGVSVDLIFCRLHLQSVPKSQELSNLDLLRGLDDTDLKCVNGTRVTRRILELVPQTKVFRMALRAVKLWAKQRALYGNIVGYPGGVAYAMMVARICQLYPRAAAPLVIWKFFYLMRKWNWPSPVLLQNHEEGSINLREWDPSIYPGDKRHLMPIITPAFPRMNACHTIGPSTKKVLLQEMERAEGIVRSIYESGRPWRDLFQRHSFFTDAYRHYICVITAGRTKEAQQAWSGLVESKVKWLIVGIEHSDAKSVELVQPYNKGFNRVHECKGDADIDKTLDGNLDCQVKEIKTVTTEQAGDVQFQAAAQTDTDGQEVPAPNGEAEVPPQTDGPQTIWTTTFYLGIGLTKGANSLDISSPIRDFTQQCKEWQNYDENLHSIRVKHMRNYDLPADVFAEGETRPTRTKKKSAPKTTDPTAMDAANKKRSFNNSGLDTLDDPAKRRASANGTATPNGVPPR

SEQ ID NO: 136 - protein, 405 aa, Schizosaccharomyces pombe
MNISSAQFIPGVHTVEEIEAEIHKNLHISKSCSYQKVPNSHKEFTKFCYEVYNEIKISDKEFKEKRAALDTLRLCLKRISPDAELVAFGSLESGLALKNSDMDLCVLMDSRVQSDTIALQFYEELIAEGFEGKFLQRARIPIIKLTSDTKNGFGASFQCDIGFNNRLAIHNTLLLSSYTKLDARLKPMVLLVKHWAKRKQINSPYFGTLSSYGYVLMVLYYLIHVIKPPVFPNLLLSPLKQEKIVDGFDVGFDDKLEDIPPSQNYSSLGSLLHGFFRFYAYKFEPREKVVTFRRPDGYLTKQEKGWTSATEHTGSADQIIKDRYILAIEDPFEISHNVGRTVSSSGLYRIRGEFMAASRLLNSRSYPIPYDSLFEEAPIPPRRQKKTDEQSNKKLLNETDGDNSE

SEQ ID NO: 137 - protein, 333 aa, T. brucei
MPPSPAVVGRSLVNSFKQFVSKDLHTRHVDATYRLVLDCVAAVDPLMRLYTFGSTVVYGVHEKGSDVDFVVLNKTDVEDGKGGDAATQVAKGLQADILAKLARVIRQKHLSWNVEEVRRTRVPVVRVKGGGAVDFDITAYRRNGVRNSALLRAYFEQNPPCRWLSMSIKRWSKQTGLNASVIGGSITSYGFNLMVVYYLLQRNHLQFVPPSTIDVSRVEPLPPHLPLEEPADEGLELGTQVLDFLHFFLHEFDSDKQVISLNRPGITTKEELDWTKSAEDFARMNGEKVHYQWCIEDPYELNLNVGRNVTPLKRDFLRRHLEKARDTALLTIV

SEQ ID NO: 138 - protein, 349 aa, S. pombe
GSHMSYQKVPNSHKEFTKFCYEVYNEIKISDKEFKEKRAALDTLRLCLKRISPDAELVAFGSLESGLALKNSDMDLCVLMDSRVQSDTIALQFYEELIAEGFEGKFLQRARIPIIKLTSDTKNGFGASFQCDIGFNNRLAIHNTLLLSSYTKLDARLKPMVLLVKHWAKRKQINSPYFGTLSSYGYVLMVLYYLIHVIKPPVFPNLLLSPLKQEKIVDGFDVGFDDKLEDIPPSQNYSSLGSLLHGFFRFYAYKFEPREKVVTFRRPDGYLTKQEKGWTSATEHTGSADQIIKDRYILAIEDPFEISHNVGRTVSSSGLYRIRGEFMAASRLLNSRSYPIPYDSLFEEA

SEQ ID NO: 139 - protein, 480 aa, T. boudieri
MSGPPSGPPSGPRYRGGHRNFGPRHLQSQERQQQQQQQQQQQQQPYQPSPQELAVLAEVVIEQIALATPSAEELERKTKLRERLRKLCTNVSPTAELEAFGSLVSGFATVGSDLDLVLVDKGGSTNRPFFEVPLLLEQELKDNGLEAQLLAKTRVPILKIKQPATEEYPKEVAADIGFANPLAIRNTDMLSMYSKCDPRVIDMVRFIKRWAKRRKINNPYKGTLSSYGYVLMVLHYLINIVEPPVLPNLQLYPIPAETPKDEITTEEGHNVWYYKDVAEIQRRVAEGTMTTNTMDLPLLLLGFFEFYAYRFGWVKDIISIRTKGGLISKVDKGWTVVAVRVGKCEAEYKDRYLFAIEDPFETNHNVSRTCNIYGVRKIRDEFKRANRIMRMREGVGALRYKLFEEAPEDVRPQHHQKQSIAGEEHVNGAPESGREDREGNQLVEELEGHSNGNGIHPSEGYPGPGLAAVVEGVQRLSVDA

SEQ ID NO: 140 - protein, 761 aa, D. stenobrocha
MELTTGDFGGPPGASNATGPGQKPEDPRTMDPVGEQTLESRLRGMLLHGGPPPVDPRTQLRHPSGLAQQAQQAQQQPIGIDPAVPGHLHQLQTAAAPPQQSRLPPSPNIHHQHLQPRLLQRSNTLPPPGGLTQPPPGLERAGTFHGPVPVAQFGYHGSYPTAHSQRGRYGNRRNGFQNAPVPQMNNNLHFPPLGTVPSQPPPPLSERSSLSNMNNPRRHSGPGHYHTDNNHSPPYKRYEAFHGAPGSPQGYSGSPPQNRQGRPYHSSRGSHGGRNNRNFWPLDYDGLTNYAKYIVESVSPTPEEIAMKDNMLRRISEICDKLVPGSRIIPFGSLVSGFATKGADMDVIFAHDTIDPAPSSHESNIPVRLANEFLKRGFEVDLLIKTRVPILKLKTPGEPVSRPGSPVSEGDGDSSEEPWPENVSCDIGFKAHLGITNSHFFRTYSHCDHRFREMVLFVKQWSKNRDLNSPYFGTLSSYGYVLMVAHFLINVVQPPVLPNLQLIPPAAETAESELMQEGFNIWYFKDLERIASGEFLPGGRNEMSLGQLVHEFFQYYTTNFNFVSECVTIRTPGGVMYKQEKGWTSARERVGEMNNTYQDRYLLALEDPFEVSHNVGRTCGGAGVRRIRAEMQRAAHIIRKVSAKEGGTPRSAGWEMPLVVEDLMTTVRETNRGFRNRRVNQKNLMEWIRNDWTVGKCLVQIDAAAEELRKKRELGLPDDAELPPESEEDSEGDEDASEEEEESSSDDGAVRVVGGGSRGGV

SEQ ID NO: 141 - protein, 337 aa, Unknown
  Phytomonas sp.
MTSLSAVNVGKLVCDSFGKLLSKNLDIKPIVQTHEIVSQSMQTVDPSMALYVFGSTAVYGFHESGCDVDFVALNQKDVSDGKAADPSSEIAKGLQVDFLSRLEASLREMHNLAWKMDLVRRTRVPVLRVKGDPCGIDFDVTARRRNGVRNSALLSAYFKQKPETRWLSMAIKQWSKRAGFNMSVDGGCLTSYGCNLMVVYYLLQRQLVKFVDPERCDVAHIPSLPSYLPLEHPAQNGSELGDMVLDFLNYYLHEFNPETEVISLSRADKTTKEMIYWTKQAEDMARISGEKVSYRWCIEDPFEHNLNVGRYVTPFKLTLLRKHMERAKETALLLSIS

SEQ ID NO: 142 - protein, 355 aa, B. saltans
MLSPAAIGKAILDHHASLIGADLSVKRVHDAHSLVKNSLDSVAPDLKLYTFGSSTVFGFHEPKSDVDFVALRQEDIVDGKGGDSTSQLAKGLQTQVLAKLAASVRQKNVQWAVEEVRRARVPVVKVKAPHIDFDITAHRRNGVRNSALLRHYLTQVPENRWLSIAIKSWSKRVGMNGPVGGYLTSYGFNILVVYYLLHRRRHEQPEVEQKEGQDGAASASVDGKQQKELTFIDKDTLDVSLIPPIPEYLALEPPNPETLGEQVLDFFDFYLSRFPMESHVISLSHKEPITKQSLNWTKTAEDMKNNTSMEKVFYRLCIEDPYEVNLNVGRNVSPFKFDLMKKHFVKGRATALGLL

SEQ ID NO: 143 - protein, 425 aa, A. deanei
MPPNPRQLGAAMIEQFRPLLQAPQQSQLTVLSEAPLFQRIQREVHELDPGARVYIFGSTRVYGFHDSGAYQQQSAQVPPSDVDMAVLRREDLLDGSGVDPSSELTRNVQAEFLEKLLNRLQQSEGSASIRSNCSSLVKRTRVPVLRVKHLVSNHSPPHDKEDNISVQVPFDFDITCGRRCGIRNSALFFHYVQQMPMIRFLLLSVKKWSKQTGLNSAIKAPYSNNNNQNHNLTSVMGGSLTSYGFHVLLLYYLLRRGVVQYIPMTVSSAAGSTDRIPNPQLDVNAIPPVPTFLPLLDPALEPHGPQGDPVRYLGELALDWCHFYLHEFDVEKEVVSLSRFDDYFADSPPSGPFPVITKDVLGWTKQREDQGRLRGEKVHYTLCIEDPYEVNLNVGRNVTSLKWMLFRKHLEKAIQTGLLLCPPSK

SEQ ID NO: 144 - protein, 432 aa, P. lactucaedebilis
MPSSSPLGATLPNSSSLAVSAAAAVVPDPLFAFTQFAVDAVRNNTVSHAELVEKERLRQELEAVLQALQPTARLVVYGSLANNLAIANCDMDLMDALHDVSVDLPALYTEALNSQGYSVKLLSKTRTPIVKVSLEHMAVPYCVDISFDNALALHNTKLIATYAACDSRVPILLMFVKLWTAVRRINDPHSGTLSSYGYALLLIFYLQNRCNSPPVLPNLQLISAMGTRSADELECSGYDIWFFKDVEKIQQAQVVKNTSSIGTLLEGFFSYFAYEFDFRDLCISIRTPGGIVTKTAKTWTQMVEHLNDRGDAKVKDRYILSIEDPFEIVHNVGRSVTRAGLYEIRGEFMAATRYVRGNKLDEILNPHRREIVAPTSSTAMHSPQQQHGTQQVSPGEGSSPTPPAVHAEARPSTGTATSTKTSQQAETVVKAE

SEQ ID NO: 145 - protein, 437 aa, S. culicis
MPPPTPMQLGRAILRHYAPSIVVPTAATSGPISSAAASTASLASIHPLFEFIQGCARRVDPQTHLYVFGSTRVYGFSEVSAPTSVGPPAALARPVKNDVDIAALSAADVATTPSPHSADAVVDKGSELAKSLQIDFLEKLKMQLQTQRQHQGDGAPPAASLSWEMEVVKRARVPVLRLQPRPAYTTSAAGGVPTYNVDVTYGRRCGVLNSALLRRYMDQQPDLRYLCLAVKRWSKLTGLNTATSPDGGGLTSYGFHLLLVYYALRRRLVAYVAPENIQWGDIAPVPAALPLRFPGDADGADAWRGRSLGERIAQDDAAAARVGEWALDFVRFYLHEFDYERDVASLSRGAAAAGLVTTEALQWTRQEEYAARGRGEYLFYRFCIEDPYEINLNVGRHMSSVKLMIFKKHLEKALETGLAFIPADEKAKQRVKPKGKE

SEQ ID NO: 146 - protein, 467 aa, B. meristosporus
MSLETDRIVSEFKDLAISQTSETNQPVPDVKVSSVEDSQHHSKAHPPKNENGNNQGHRHGKKPFHRGSTFGEQGKSDRSYGARSFHPNRGTGQYEGKRVLDKSDPRYLKARARFISVLTEQVSELYLKLTPSVEEVSRREQLYFRIHSICEELFPDAQLFQFGSTANGFGLKNADVDFCLCTAENSSLTAVAFVEQLGSVLKERGMENVMLLTRTRIPIIKLKDPVSELNCDIGFNNRLAVYNTRLLRMYAEVDSRVKEIVAIVKHWAKSRQINEPYLGTLSSYAYVLMIIFVLQQRGVLPCLQQICEGEKKEVLVDNYDAYFFDNITDLPKYWKSQNNESLGELLYEFFRFYASDFSYYGSVVSVRTGQVMSKEEKGWTAEATRESGASHVQDRYWVCIEDPFEVTHNLGRPVNKNSLYTIRGEFMRAANILGGTRTSTILERLCQVYVPEEEPPRAPPAPHERSN

SEQ ID NO: 147 - protein, 354 aa, N. californiae
MKTTERTCTFEFYDELTEEINKIVNKIKAKPNDLKKRENLFNYIKLVAKKIFPNSKAYKYGSIENGFSLTNGDIDICILNNKEFNNTPAECVEILGEKLKEDGINDIKLLIRARVPIIKMKDNKSGISCDIGFQNKLAIQNTKLINAYSKIDDRFKQMVFIIKYWSKMKNINEPYMGTLSSYCFILMIIHFLQIKEPPVLPNLQKIYLDNFIEYEYIDDFNVSFFENIDELHKYWNSKNNESLGELLVEFFKYYANDFPYITGVASIRVGNIITKEEKKWTREHQFEINKSNSVKDRYWFCVEDPFEITHNLGRPVDRKSLFRIRGEFLKAVKIINNKNLTALQVLEKLLEKNI

SEQ ID NO: 148 - protein, 344 aa, Unknown
  Perkinsela sp.
MLDPSETCMGRIKNIHAIITSKLMGLLVEKNAQIYCFGSSMVNGVVEKKSDVDVVYLTREDLQKPLSIGDVCNPQSRSEQTSILSAISKILMKDSELFSTVQEKPRARVPYVRGVLKNGLEIDISAHRRNGVRNSLLLRSYFSQIPPTRPSLPNATTSVYRMLSLALKFWGKRTGLVDPVQTFLTSYAFNVLICYYLQQRGGMDFIHPESILIPKGHPTVPDYREIALAGTQGHTCLGWYMRDFLKFYNHEFDYNNTVVSLSRKGITTKEYLGWGLRDEERMHGTDGNAFFYRFCVEDPYENRLNLGRFVTPLRYSMFRMALHQAQLNGFGYLNLKNYGAKIVD

SEQ ID NO: 149 - protein, 352 aa, S. complicate
MMAHPSQGGAHPLSAGPTNPQPYQDAYSGFTEYVLSINRPQYPSANEMYLKEQLRSLIDVEIVQRIQPSARVIPFGSLVNGFATANSDLDLCILDDSPNPRYKIKTELPEVLAAEFERYGFEVKLLTKTRIPIIKLVQGPTAQFPLGLAMDIGFENRLALHNTRLLATYSRIDSRLREMVLFVKHWAKVRGINSSYHGTLSSYGYVLTILHFLINVASPSCLPNLQHIGAQVPVPFEELECEGYNIWFFKDLTNIPPSLNRRSIGELMAEYFSYYAQFDFRNMVISIRTPGGLLTKTSKGWTQALDRVGPEDQKVRSRNFLCIEDPFEITHNVGRTVGKNGLYDVWPFVSFA

SEQ ID NO: 150 - protein, 388 aa, S. ochraceum
MATVFAQSHSLLNTRQPHQHVQQPAQQPPQQQQQQQSTRPKQLSKPRFYAEFSQCLFDFVIQLLPTPEELAIKEDVRKLLERLIRTLEPDSRLLSFGSTANGFSLRNSDMDLCCLIDSDDRLAASDLVTMLGDLLERETKFHVKPLPYARIPIVKLSLDPSPGLPFGIACDIGFENRLALENTRLLMCYAMIDPARVRTMVLFLKVWCKRRKINSPYKGTLSSYGYVLLVIFFLVHVKNPPVLPNLQSMPPLRPMDKEESTLNGHNVWFFDDINLLRQRWQSTNTESVAELLVDFFKFYSREFPYNSGVVSIRAGGIKKDSKGWFSEAERGSARERNRLCIEDPFETDFNVARCVTRDGLYTIRGELMRASRILAARPERPIVALAQL

SEQ ID NO: 151 - protein, 421 aa, G. androsaceus
MAAYSPTMSSNQHYHELNSASPPARRKWLADFSECLFSFVIQLLPTQEELAVKEDVRKLLERLIRTIEPESRLLSFGSTANGYGLRNSDMDLCCLIDSEERLAASDLVTMLGDLLERETKFHVKPLPHARIPIVKLSLDPSPGLPLGIACDIGFENRLALENTRLLMCYAMIDPTRVRTMVLFLKVWSKRRKINSPYKGTLSSYGYVLLVIYFLVHVKNPPVLPNLQQMPPLRPIPKEETYLGGFNTWFFDDIELLRQRWHSENTETVAELLIDFFRYFSRDFPYGVGVASIRAGLLKKDSKGWQNDLSASRYNDARERNRFCIEDPFETDYNVARCVTKDGLYTIRGEFMRASRVLAARPERAILALADLCDERKDEDPCRRASLYSFSSSAPTNTVFGGKSINAFQRNTDYSLSFDQTP

SEQ ID NO: 152 - protein, 512 aa, T. equiperdum
MGVRLYSCDACPHAVFTTHAALLAHAEEHHADLLPDHARLRRIAQKLNPVWNRALNARRNTITSWGKKIFHVAAQRDAGESKMQEAHRARAQLECVVRRWHDKARVFIFGSSVAMGVWDGTADIDFAVVDVDAMERGSWPPLEKNAVRSITELLRRVGFSFVNLEPISHARVPIIKHHASSPILTVARRDAEDVVARSIRFILNGPATREDRLLLEGSVRDAVGPTGVQQVWWNRTSDMMSATLESTTAAVRAAMCSPALASASLRTKVQPAHDECRPELYNIDFALSFRAFGIRNSTLLRKYLLSHPCARPGAIVLKDWSKTSGVNNSVNGYFTSYAINIMWIYYLVQKGYVPYVDPLEIPESLVNYTDFDPRYTPMIDPEITNTEREELYKAAGDMLVGFFYFYSFEFDWGHNVISLNRPGITTKRMLGWHVEDVVPVASTSVSSGGGGSNVKRHPTRYELCIEDPYEENLNLGRHIGVTKSLRVRTELYRGLLSLLKEGETRSCVFAAA

SEQ ID NO: 153 - protein, 531 aa, M. conica
MTAGRDIRSPPRGQRGQGFNKSQNSPRALPLNALDHFPPLGTQPTKAKTPPPNYSQQQQSTTAKPQQRTQYQPLPIDTRNTNPHHARGEVHGRRNFRGDYNQRQLRRDIGPEVCQYLSELSKKVIAEAAPPESEILVKRALLERLEAISKTIVPDAKLIAFGSLVTGFATANSDLDVIFTGGDRVDILNESSDDPSSNFRIPMLLESKLQEEGFGTTLLTKTRVPILKLVQKATEQSPYELQCDIGFSNHLALYNTQMLLTYSKCDPRVKEMMIFIKWWAKRRHINNPYRGTLSSYGYALIVLHYLINVVKPPVLPNLQTFPVPDSAPTNEIIFEGDSDDTFEIWFYKDIEKLPKSDNAMDIGELLKGFFEYYAHNFQWGREVVSIRTKGGLMTKQEKGWVAAVIKPGRTENSEVKNRYLFAVEDPFETEHNVSRTCNGPGVNRIKDEFKRAVWLIRVRDGGKTLFQNLCMEAPPERVWVRREDRDRRGIEEAGGHGSKDHQGAEGEAGDGVKHESVVLSEDGRNALESLV

SEQ ID NO: 154 - protein, 474 aa, P. murina
MDSVKGFEGVESVVFQKEMKKLTKISDFSGKNYGSGNEIEDLKKSLEKMMLESVNIQEKGVFLREKGSEGSYEANLNDKDVLKNNKKDSNFRNIKYYEPKDYGLYVNGKAVWRKNYDEYFVLSTFISHTLKHIMPTNEEISQKERFRFFLSEILNTCRPSAKLVLFGSVASGLAIVNSDMDFCVMDDSLDLHTDEFLKIFSEEIKKYGMETTLLFRTRVSIIKVNSKGSFNFPQGISCDIGFNNKLAIYNTKLLATYSKCDHRVRKIILFVKYWAKRRKINDPYHGTLSSYGYVLLILHYLINIVPIPLLPNLQQMKVASWRSIPQSEIECDGYNVWFYKDIDISCSFSNNTDSLGKLVYGFFYYYAYQFNWKDHVVSIRTQTGLLTKQEKGWTQAMERVFVSDKIFKDRYILAIEDPFEITHNVGRTVNKQSSHIIRGEFFRASKLSSSKLRKKIFNELCEERIIINNFSNVI

SEQ ID NO: 155 - protein, 445 aa, S. japonicus
MSGNGNGHFIPGVHTVEEIERQFVNLALQKRNQAAAAAAAAAERELDPVTCFLLSTYDDVRVSDDELREKDAIMNLLKHVVHSVRPEADIVAFGSIQSGLALKNSDIDACILLPDIGEEMEEFASECFERFTALGFEGKYLRKARIPIIKLLSDTKNRYYYGFQCDIGFNNQLAIYNTSLLHQYSLIDPRCKQLAILVKYWAKQKRINSPYYGTLSSYGYVLMVLFYLIHVVRPAVLPNLQDSPHKQDLYVEGFNVGFVRGTTVARRNTESLPQLLAGFYGFFAHEFNYRESVISIRQPGGLLKKVDKDWTLAKEHTGSADQVIKDRYVLAIEDPFEITHNVGRTVSKAGLFEIRGEFMQATRLLNAATLHSTSAIRKLRASLFKERLEPKSHLKYQKALRQKRMGTEGKAAAEGKTNNPTAASGSDSAPSESASRTATVESREP

SEQ ID NO: 156 - protein, 620 aa, A. nigricans
MNPPQQTTGLEDHLRSLIINNQTPPNHSISPAAKIPHKHHPRTSTDSRGFVRSQTIHRPGPAASQPPSNGPNRRFPGEAQRPDHRPPHLRGPSQNRNYWHGAPPPPRQVVPQGLSPPAATNLHHFPPLGAKVAPPPPSLPSYHSVPTAPCHRPENQRPVYQNDNRDSYHYKQRGLYDGYLAEQWRELDKMAKEVLLTATPVDGEKEAKEKFMQDLEAVCQKVEPTAKLIPFGSIVTGFATRGSDIDCVFTSNTDSALQNDKITEPETPFDRHDELVTQLQESGYNAQLLTRTRVPIIKLVRPATPDVPDSVSCDIGFNNFLAIHNTRLLRTYAACDERLVQMVLFIKWWAKRRHINSPYRGTLSSYGYALLIVHYLINIAQPPVLPNLQLFHPPATQTSTELQYEHNGNVCNIWYLKDTSTLPRSANKASIGELLRGFFEYYAFTFRWQNDVVSIRTAGGILTKMEKNWCAARSRPGGREEGQVWEVKDRVDVQHRYLLAIEDPFETTHNVARTCTFNGVGRIKSELKRAMYLIKSRDPGHHRLRTELFEEAPPERPFVRNGKEVVDKLEKVEIVEERAGVAVEIVEERAGVAVEIVEERAGVALEVVEERAGAAVEVDV

SEQ ID NO: 157 - DNA, 18 bp, Artificial Sequence
  polyC initiator
  misc_feature (17)..(17): n is deoxyinosine
ccccccccccccccctnt

SEQ ID NO: 158 - DNA, 12 bp, Artificial Sequence
  non-polyC initiator
  misc_feature (11)..(11): n is deoxyinosine
ttttttttttnt

SEQ ID NO: 159 - DNA, 12 bp, Artificial Sequence
  initiator
ccctccctccct

SEQ ID NO: 160 - DNA, 15 bp, Artificial Sequence
  initiator
ccctcccctccccct

(19) United States
(12) Patent Application Publication
(10) Pub. No.: US 2025/0283132 A1
(43) Pub. Date: Sep. 11, 2025

(54) METHODS AND KITS FOR ENZYMATIC SYNTHESIS OF G4-PRONE POLYNUCLEOTIDES

(71) Applicant: DNA SCRIPT, Le Kremlin-Bicêtre (FR)

(72) Inventors: Gabriel DE CROZALS, Le Kremlin-Bicêtre (FR); Emmanuel DE REVEL, Le Kremlin-Bicêtre (FR); Henri LACHAIZE, Le Kremlin-Bicêtre (FR); Adrian HORGAN, Le Kremlin-Bicêtre (FR); Xavier GODRON, Le Kremlin-Bicêtre (FR)

(73) Assignee: DNA SCRIPT, Le Kremlin-Bicêtre (FR)

(21) Appl. No.: 18/285,014

(22) PCT Filed: Apr. 1, 2022
(86) PCT No.: PCT/EP2022/058814

§ 371 Date: Sep. 29, 2023

(30) Foreign Application Priority Data
Apr. 2, 2021 (EP) EP21305428

(51) Int. Cl. C12P 19/34; C12N 9/12

(52) U.S. Cl. CPC C12P 19/34; C12N 9/1264; C12Y 207/07031

(57) ABSTRACT

The present invention is directed to methods, compositions and kits for template-free enzymatic synthesis of polynucleotides having sequences capable of forming G-quadruplex (G4) structures. In accordance with the invention elongation reactions affected by G4 formation are carried out in the presence of polyC oligonucleotides, such as polyC initiators, that inhibit or prevent formation of either intra-strand or inter-strand G4 structures.

### BACKGROUND

Interest in enzymatic approaches to polynucleotide synthesis has recently increased not only because of increased demand for synthetic polynucleotides in many areas, such as synthetic biology, CRISPR-Cas9 applications, and high-throughput sequencing, but also because of the limitations of chemical approaches to polynucleotide synthesis, such as upper limits on product length and the use and the need to dispose of organic solvents, Jensen et al, Biochemistry, 57: 1821-1832 (2018). Enzymatic synthesis is attractive because of its specificity and efficiency and its requiring only mild aqueous-compatible reagents and reaction conditions.

Currently, most enzymatic approaches for both DNA and RNA synthesis employ template-free polymerases to repeatedly add 3′-O-blocked nucleoside triphosphates to a single stranded initiator or an elongated strand attached to a support followed by deblocking until a polynucleotide of the desired sequence is obtained, e.g. Hiatt and Rose, International patent publication WO96/07669. The inventors have discovered that template-free polymerases, such as, terminal deoxynucleotidyltransferases (TdTs), do not efficiently couple nucleotides to elongated strands at sequences that are capable of forming G-quadruplexes (G4s). G4 structures are commonly occurring secondary structures important for their involvement in a variety natural processes, e.g. Kwok et al, Trends in Biotechnology, 35(10): 997-1013 (2017); Murat et al, Curr. Opin. Genet. Devel., 25: 22-29 (2014); and the like. Thus, the state of the art of enzymatic synthesis currently is deficient in its capability to synthesize polynucleotides prone to forming G4 structures.

In view of the interest in extending the application of template-free enzymatic synthesis of polynucleotides, the field would be advanced if methods were available to increase the efficiency and yield of target polynucleotide containing G4 structures.

### SUMMARY OF THE INVENTION

The present invention is directed to methods and kits for template-free enzymatic synthesis of either DNA or RNA polynucleotides that employ agents in a synthesis reaction for disrupting the formation of G4 structures. In one aspect, such agents are polycytidylate or polydeoxycytidylate oligonucleotides (collectively referred to herein as “polyC oligonucleotides”). In some embodiments, polyC oligonucleotides are components of initiators and/or synthesis supports such that they are capable of interacting with G-rich domains of polynucleotides being synthesized. In other embodiments, polyC oligonucleotides are free in solution during selected coupling or elongation steps during synthesis.

In some embodiments, the invention is directed to methods of synthesizing a polynucleotide having a predetermined sequence capable of forming a G4 structure, wherein the method comprises the steps of: (a) providing, attached to a synthesis support, initiators each with a free 3′-hydroxyl; (b) repeating in a reaction mixture including the synthesis support, until the polynucleotide is formed, cycles of (i) contacting under elongation conditions the initiators or elongated fragments having free 3′-O-hydroxyls with a 3′-O-blocked nucleoside triphosphate and a template-independent polymerase so that the initiators or elongated fragments are elongated by incorporation of a 3′-O-blocked nucleoside triphosphate to form 3′-O-blocked elongated fragments, and (ii) deblocking the elongated fragments to form elongated fragments having free 3′-hydroxyls, wherein the reaction mixture for elongating the initiators or elongated fragments comprise polyC oligonucleotides capable of forming duplexes with regions of the polynucleotide. In some embodiments, initiators each comprise a segment consisting of a polyC oligonucleotide. In other embodiments, a synthesis support is provided having polyC oligonucleotides attached thereto, eventually in addition to initiators. In still other embodiments, polyC oligonucleotides are in solution as a component of an elongation reaction mixture for selected elongation cycles in which G4 structure formation has a high likelihood of occurrence. In some embodiments, such selected elongation cycles may be determined by a conventional G4 prediction algorithm.

### BRIEF DESCRIPTION OF THE DRAWINGS

FIG. 1 diagrammatically illustrates a method of template-free enzymatic synthesis of a polynucleotide.

FIGS. 2A-2D diagrammatically illustrate various embodiments for including in a reaction mixture polyC oligonucleotides.

### DETAILED DESCRIPTION OF THE INVENTION

The general principles of the invention are disclosed in more detail herein particularly by way of examples, such as those shown in the drawings and described in detail. It should be understood, however, that the intention is not to limit the invention to the particular embodiments described. The invention is amenable to various modifications and alternative forms, specifics of which are shown for several embodiments. The intention is to cover all modifications, equivalents, and alternatives falling within the principles and scope of the invention.

The practice of the present invention may employ, unless otherwise indicated, conventional techniques and descriptions of organic chemistry, molecular biology (including recombinant techniques), cell biology, and biochemistry, which are within the skill of the art. Such conventional techniques may include, but are not limited to, preparation and use of synthetic peptides, synthetic polynucleotides, monoclonal antibodies, nucleic acid cloning, amplification, sequencing and analysis, and related techniques. Protocols for such conventional techniques can be found in product literature from manufacturers and in standard laboratory manuals, such as Genome Analysis: A Laboratory Manual Series (Vols. I-IV); PCR Primer: A Laboratory Manual; and Molecular Cloning: A Laboratory Manual (all from Cold Spring Harbor Laboratory Press); Lutz and Bornscheuer, Editors, Protein Engineering Handbook (Wiley-VCH, 2009); Hermanson, Bioconjugate Techniques, Second Edition (Academic Press, 2008); and like references.

The invention is directed to improvements to template-free enzymatic synthesis of polynucleotides, especially DNA or RNA, which permit higher yields of long polynucleotides by providing synthesis conditions that suppress or disrupt the formation of G-quadruplex (or G4) secondary structures in growing chains. Without the intention of being limited to a particular theory or hypothesis, it is believed that the formation of G4 structures limits access to synthesis reagents, such as template-free polymerases, thereby inhibiting chain extension, or elongation, and thereby increasing the variability of product length. In part, the invention is based on a recognition and appreciation that the negative effects of such secondary structures on product yield can be mitigated or suppressed by providing elongation (or extension or coupling) conditions that include agents, particularly polyC oligonucleotides, which disrupt the formation of G4 structures. In particular, it is believed that such disruption occurs by providing alternative stable configurations, e.g. duplexes with polyC regions, that a growing strand having G-rich regions can occupy. In view of the above, in some embodiments, initiators of the invention may comprise polyG oligonucleotides wherein an alternative stable configuration may be a G4 structure between the polyG oligonucleotide in the initiator and G-rich sequences of the polynucleotide being synthesized. Such polyG oligonucleotides may have a length in the range of from 2 to 20 guanylates or deoxyguanylates.

G-quadruplex structures are common in nature and may be predicted using available algorithms, e.g. Lombardi et al, Nucleic Acids Research, 48(1): 1-15 (2020), and like references. G₃₊N₁₋₇G₃₊N₁₋₇G₃₊N₁₋₇G₃₊ is a common G4 motif, where “N” is any nucleotide and “3+” means 3 or more G's in a row. As used herein, the term “G4-prone polynucleotide” means a polynucleotide having a nucleotide sequence that can form a G4 structure under elongation reaction conditions. In some embodiments, “G4-prone polynucleotide” means a polynucleotide having a nucleotide sequence that a conventional G4 prediction algorithm indicates as likely to form a G4 structure, e.g. Burge et al, Nucleic Acids Research, 34(19): 5402-5415 (2006); Huppert et al, Nucleic Acids Research, 33(9): 2908-2916 (2005); Kwok et al, Trends in Biochemistry, 35(10): 997-1013 (2017); Lombardi et al (cited above); Murat et al, Curr. Opin. Genetic & Development, 25: 22-29 (2014); Todd et al, Nucleic Acids Research, 33(9): 2901-2907 (2005); Bedrat et al, Nucleic Acids Research, 44(4): 1746-1759 (2016); and the like.

PolyC oligonucleotides of the invention may have lengths of 2 or more nucleotides. In some embodiments, polyC oligonucleotides of the invention have lengths in the range of from 2 to 60 nucleotides, or from 2 to 50 nucleotides, or from 2 to 40 nucleotides, or from 2 to 30 nucleotides, or from 2 to 20 nucleotides. In other embodiments, polyC oligonucleotides of the invention have lengths in the range of from 6 to 60 nucleotides, or from 6 to 50 nucleotides, or from 6 to 40 nucleotides, or from 6 to 30 nucleotides, or from 6 to 20 nucleotides. In some embodiments, initiators of the invention have a length in the range of from 6 to 50 nucleotides and polyC oligonucleotides make up fifty percent or more of the initiator sequence. In some embodiments, polyC oligonucleotides of the invention have lengths, concentrations and/or configurations (i.e. segment of initiator, independently attached to same solid support as initiator, or in free solution) of sufficient magnitude to increase the purity of the synthesized polynucleotides by 20 percent or more as compared to an equivalent synthesis using a polyT initiator.

In some embodiments, a plurality of polyC oligonucleotides may be present in a larger oligonucleotide, such as an initiator, which is a component of an elongation reaction mixture. For example, an initiator may have a segment comprising several polyC oligonucleotides separated by a non-C nucleotide, e.g. -CCCTCCCTCCCT- (SEQ ID NO: 159), -CCCTCCCCTCCCCCT- (SEQ ID NO: 160), or the like. In some embodiments, such composites of polyC oligonucleotides may be selected for ease of manufacturing.

Whenever initiators (described more fully below) comprise polyC oligonucleotides, the polyC oligonucleotides may comprise all or a portion of the initiators. In different embodiments, polyC oligonucleotides may be provided in any or all of the following configurations: (i) as part of initiators, (ii) as part of oligonucleotides attached to the same synthesis support as initiators, and (iii) as part of oligonucleotides in free solution. In each case the part of an oligonucleotide or initiator that is polyC may be the entire oligonucleotide or initiator. In regard to (ii), such oligonucleotides may be attached to a synthesis support by either a 5′ end or a 3′ end. In some embodiments, whenever an oligonucleotide of (ii) is attached by a 5′ end its 3′ end is capped so that nucleotides are not attached to it during coupling or elongation steps. Likewise, in regard to (iii), polyC oligonucleotides in free solution have their 3′ ends capped so that nucleotides are not attached to them during coupling or elongation steps.

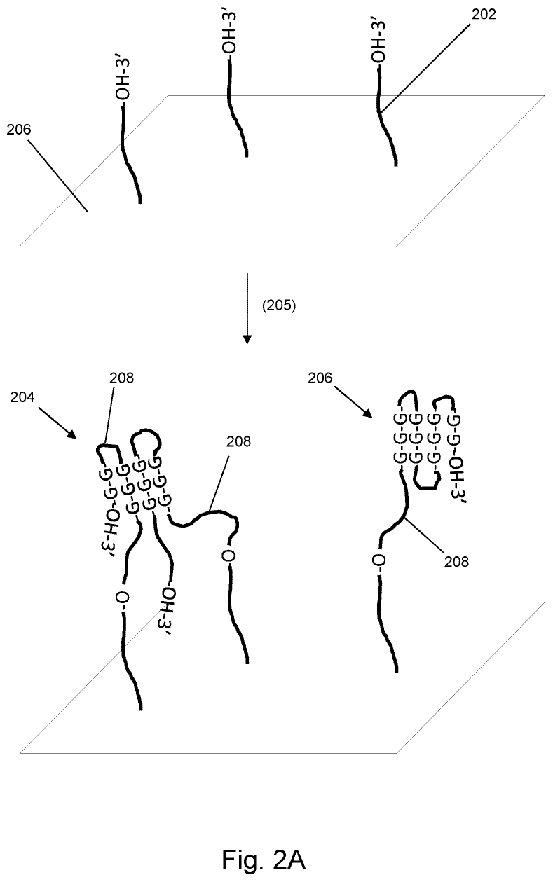

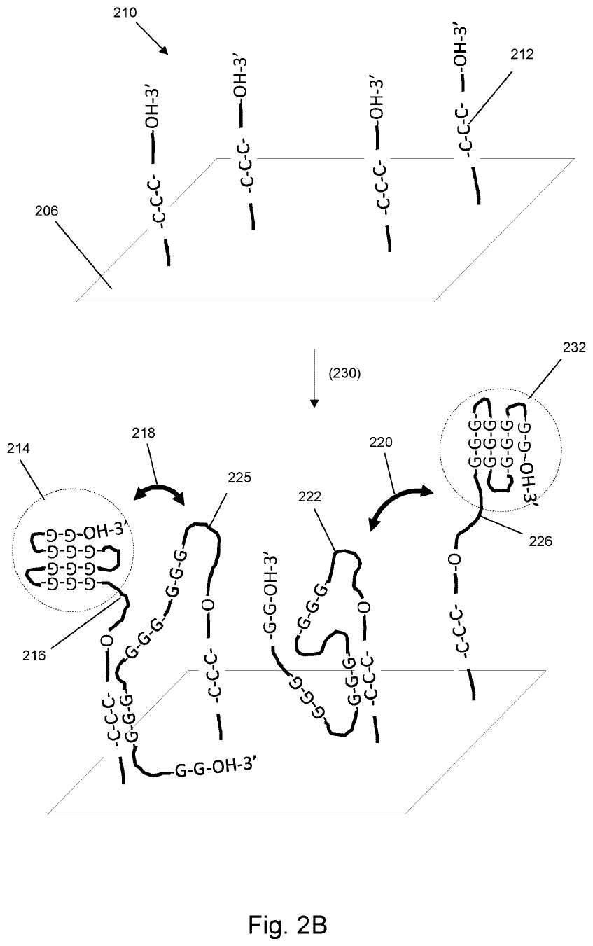

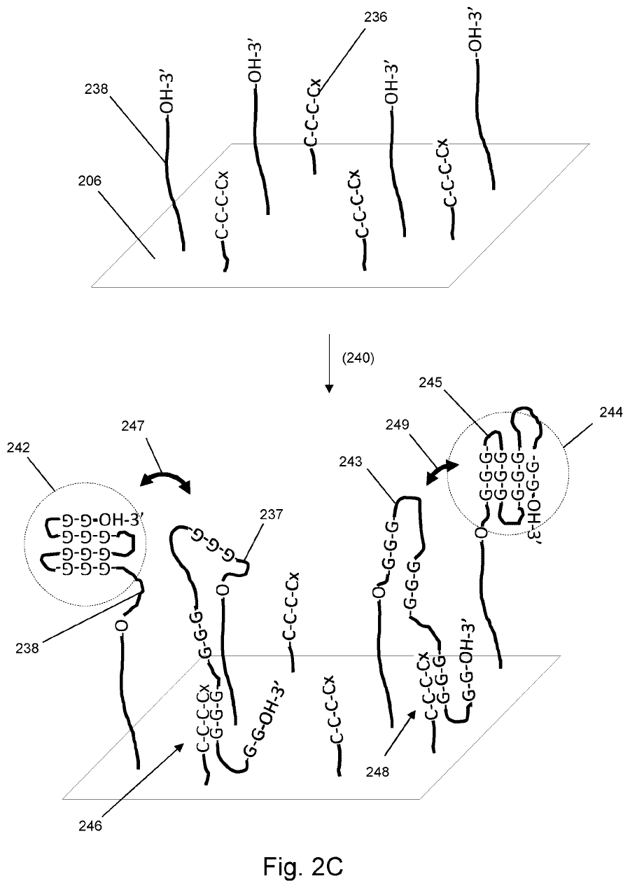

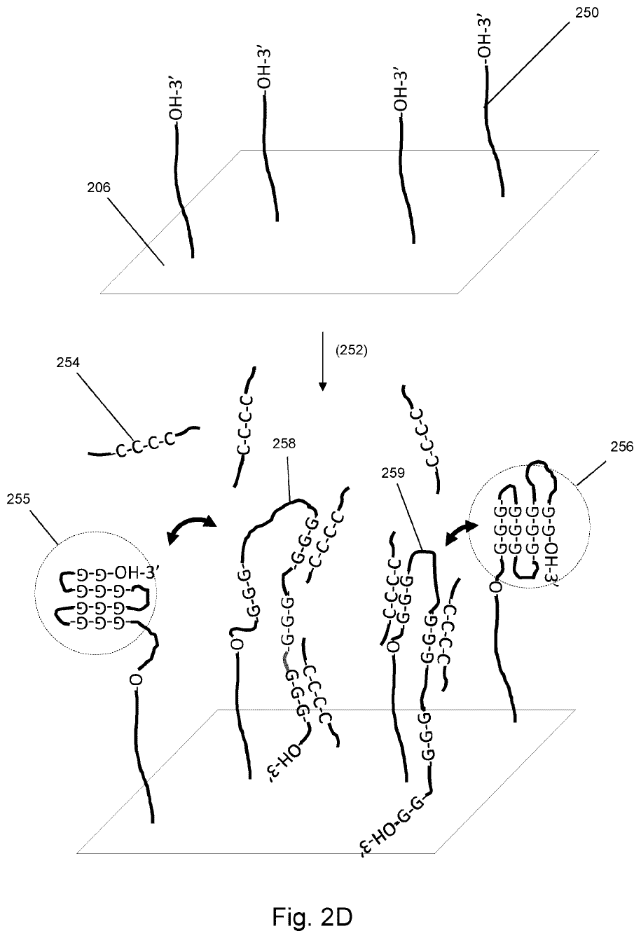

FIGS. 2A-2D illustrate various aspects of the invention. FIG. 2A illustrates synthesis support (200) with oligonucleotide initiators (202) attached by their 5′ ends to support (200). After synthesis (205) of polynucleotides (208) containing polyG segments, either inter-strand (204) G4 structures may form or intra-strand (206) G4 structures may form to inhibit further extension of the polynucleotides. In some cases (e.g. 206) inhibition does not occur until the synthesis of the final G-rich segment of the polynucleotides takes place, which allows G4 formation. Thus, in some embodiments, free solution polyC oligonucleotides need be introduced into coupling reactions only in selected coupling steps which may be determined for particular polynucleotides using G4 prediction algorithms such as described in Lombardi et al (cited above). FIG. 2B illustrates embodiment (i) of the previous paragraph in which polyC oligonucleotides are components of initiators. In the upper panel of FIG. 2B initiators (210) are attached to synthesis support (206). Each initiator contains a polyC oligonucleotide segment (212). After synthesis up to where an intra-strand G4 structure starts to form, the bottom panel of FIG. 2B shows three possible configurations of the polynucleotides in their interactions with themselves and each other. Strands (216) and (226) illustrate at (214) and (232), respectively, G4 structures at their 3′ ends, which inhibit a template-free polymerase, such as a TdT, from participating in an extension reaction. Stand (222) illustrates a polynucleotide in which one of its polyG segments forms an intra-strand duplex with the polyC component of the initiator, which thereby inhibits the formation of a G4 structure at its 3′end. Strands (216) and (225) illustrate the formation of an inter-strand duplex between the polyC component of the initiator of strand (216) and one of the polyG segments of strand (225), thereby disrupting the formation of a G4 structure at the end of strand (225). As mentioned above, it is believed that because of the formation of the G-C duplexes and the transitions (e.g. (218) and (220)) of the strands between duplex states and G4 states, template-free polymerases have an opportunity to interact with free 3′-hydroxyls of the growing chains for catalyzing a coupling reaction that otherwise would occur with much lower efficiency.

FIG. 2C illustrates an embodiment in which polyC oligonucleotides (236) are provided as separate oligonucleotides attached to the same synthesis support (206) as initiators (238). PolyC oligonucleotides may be attached by either their 5′ ends or their 3′ ends; however, if attached by their 5′ ends, preferably the 3′ ends are capped (indicated in the figure as an “x”) so that they are not extended during extension steps. The initiators and polyC oligonucleotides may be attached using conventional attachment chemistries. Typically both would have reactive moieties (e.g. amines) on their attachment ends and would be reacted with complementary moieties on the synthesis support in relative concentrations selected so that the density of polyC oligonucleotides were high enough to permit inter-strand duplexes to form. The bottom panel of FIG. 2C shows the interaction of polyC and polyG regions after synthesis (240) up to the point where intra-strand G4 structures, e.g. (242) and (244), can form. In this embodiment, only inter-strand C-G duplexes, e.g. (246) and (248), between strands being synthesized and the polyC oligonucleotides. As above, the polyC oligonucleotides in proximity to the synthesized strands (237, 238, 243, 245) permit transitions, e.g. (247) and (249), between duplex states, (246) and (248) and G4 states (242) and (244), respectively, which allow more efficient extension reactions to take place.

FIG. 2D illustrates an embodiment in which polyC oligonucleotides are provided in solution as a component of a reaction mixture. In the upper panel of FIG. 2D, synthesis support (206) is illustrated with initiators (250) and no polyC oligonucleotides. After synthesis (252) of target polynucleotides up to the point in which intra-strand G4 structures begin to form, e.g. (255) and (256), a plurality of extension steps may be performed in reaction mixtures that contain polyC oligonucleotides (254). As shown for stands (258) and (259) the polyC oligonucleotides in solution form duplexes with polyG segments that otherwise would contribute to G4 structures.

### Template-Free Enzymatic Synthesis of DNA

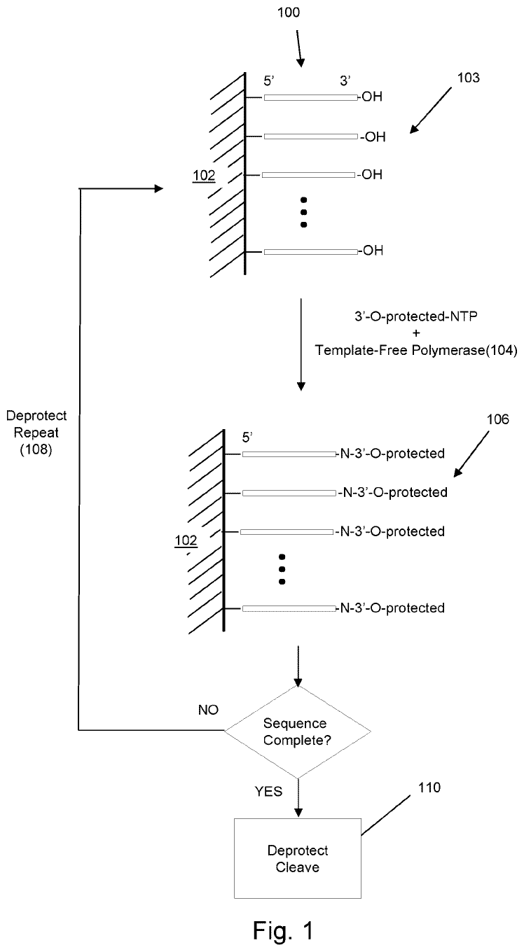

Generally, methods of template-free (or equivalently, “template-independent”) enzymatic DNA synthesis or RNA synthesis comprise repeated cycles of steps, such as illustrated in FIG. 1, in which a predetermined nucleotide is coupled to an initiator or growing chain in each cycle. The general elements of template-free enzymatic synthesis of polynucleotides is described in the following references: Ybert et al, International patent publication WO/2015/159023; Ybert et al, International patent publication WO/2017/216472; Hyman, U.S. Pat. No. 5,436,143; Hiatt et al, U.S. Pat. No. 5,763,594; Jensen et al, Biochemistry, 57: 1821-1832 (2018); Mathews et al, Organic & Biomolecular Chemistry, DOI: 0.1039/c6obO1371f (2016); Schmitz et al, Organic Lett., 1(11): 1729-1731 (1999).

Initiator polynucleotides (100) are provided, for example, attached to solid support (120), which have free 3′-hydroxyl groups (130). To the initiator polynucleotides (100) (or elongated initiator polynucleotides in subsequent cycles) are added a 3′-O-protected-dNTP or 3′-O-protected-rNTP and a template-free polymerase, such as a TdT or variant thereof usually for DNA synthesis (e.g. Ybert et al, WO/2017/216472; Champion et al, WO2019/135007) or a polyA polymerase (PAP) or polyU polymerase (PUP) or variant thereof usually for RNA synthesis (e.g. Heinisch et al, WO2021/018919) under conditions (140) effective for the enzymatic incorporation of the 3′-O-protected-NTP onto the 3′ end of the initiator polynucleotides (100) (or elongated initiator polynucleotides). This reaction produces elongated initiator polynucleotides whose 3′-hydroxyls are protected (106). If the elongated sequence is not complete, then another cycle of addition is implemented (108). If the elongated initiator polynucleotide contains a competed sequence, then the 3′-O-protection group may be removed, or deprotected, and the desired sequence may be cleaved from the original initiator polynucleotide (110). Such cleavage may be carried out using any of a variety of single strand cleavage techniques, for example, by inserting a cleavable nucleotide at a predetermined location within the original initiator polynucleotide. An exemplary cleavable nucleotide may be a uracil nucleotide which is cleaved by uracil DNA glycosylase. If the elongated initiator polynucleotide does not contain a completed sequence, then the 3′-O-protection groups are removed to expose free 3′-hydroxyls (103) and the elongated initiator polynucleotides are subjected to another cycle of nucleotide addition and deprotection.

As used herein, the terms “protected” and “blocked” in reference to specified groups, such as, a 3′-hydroxyls of a nucleotide or a nucleoside, are used interchangeably and are intended to mean a moiety is attached covalently to the specified group that prevents a chemical change to the group during a chemical or enzymatic process. Whenever the specified group is a 3′-hydroxyl of a nucleoside triphosphate, or an extended fragment (or “extension intermediate”) in which a 3′-protected (or blocked)-nucleoside triphosphate has been incorporated, the prevented chemical change is a further, or subsequent, extension of the extended fragment (or “extension intermediate”) by an enzymatic coupling reaction.

As used herein, an “initiator” (or equivalent terms, such as, “initiating fragment,” “initiator nucleic acid,” “initiator oligonucleotide,” or the like) refers to a short oligonucleotide sequence with a free 3′-hydroxyl at its end, which can be further elongated by a template-free polymerase, such as TdT. In one embodiment, the initiating fragment is a DNA initiating fragment. In an alternative embodiment, the initiating fragment is an RNA initiating fragment. In some embodiments, an initiating fragment possesses between 3 and 100 nucleotides, in particular between 3 and 20 nucleotides, which may be all or partially polyC. In some embodiments, the initiating fragment is single-stranded. In alternative embodiments, the initiating fragment may be double-stranded. In some embodiments, an initiator oligonucleotide may be attached to a synthesis support by its 5′end; and in other embodiments, an initiator oligonucleotide may be attached indirectly to a synthesis support by forming a duplex with a complementary oligonucleotide that is directly attached to the synthesis support, e.g. through a covalent bond. In some embodiments a synthesis support is a solid support which may be a discrete region of a planar solid, or may be a bead.

In some embodiments, an initiator may comprise a non-nucleic acid compound having a free hydroxyl to which a TdT may couple a 3′-O-protected dNTP, e.g. Baiga, U.S. patent publications US2019/0078065 and US2019/0078126.

Synthesis supports to which PolyC-containing initiators are attached may comprise polymers, porous or non-porous solids, including beads or microspheres, planar surfaces, such as a glass slide, membrane, or the like. In some embodiments, a solid support, or synthesis support, may comprise magnetic beads, particle-based resins, such as agarose, or the like.

Synthesis supports include, but are not limited to, soluble supports, such as, polymer supports, including polyethylene glycol (PEG) supports, dendrimer supports and the like; non-swellable solid supports, such as, polystyrene particles, Dynabeads, and the like; swellable solid supports, such as resins or gels, including agarose. Synthesis supports may also form part of reaction chambers, such as, the filter membrane of a filter plate. Guidance for selecting soluble supports is found in references Bonora et al, Nucleic Acids Research, 212(5): 1213-1217 (1993); Dickerson et al, Chem. Rev. 102: 3325-3344 (2002); Fishman et al, J. Org. Chem., 68: 9843-9846 (2003); Gavert et al, Chem. Rev. 97: 489-509 (1997); Shchepinov et al, Nucleic Acids Research, 25(22): 4447-4454 (1997); and like references. Guidance for selecting solid supports is found in Brown et al, Synlett 1998(8): 817-827; Maeta et al, U.S. Pat. No. 9,045,573; Beaucage and Iyer, Tetrahedron, 48(12): 2223-2311 (1992); and the like. Guidance for attaching oligonucleotides to solid supports is found in Arndt-Jovin et al, Eur. J. Biochem., 54: 411-418 (1975); Ghosh et al, Nucleic Acids Research, 15(13): 5353-5372 (1987); Integrated DNA Technologies, “Strategies for attaching oligonucleotides to solid supports,” 2014 (v6); Gokmen et al, Progress in Polymer Science 37: 365-405 (2012); and like references.

In some embodiments, the solid-phase support will typically be comprised of porous beads or particles in the form of a resin or gel. Numerous materials are suitable as solid-phase supports for the synthesis of polynucleotides. As used herein, the term “particle” includes, without limitation, a “microparticle” or “nanoparticle” or “bead” or “microbead” or “microsphere.” Particles or beads useful in the invention include, for example, beads measuring 1 to 300 microns in diameter, or 20 to 300 microns in diameter, or 30 to 300 microns in diameter, or beads measuring larger than 300 microns in diameter. A particle comprising polyC-containing initiators can be made of glass, plastic, polystyrene, resin, gel, agarose, sepharose, and/or other suitable materials. Of particular interest are porous resin particles or beads, such as, agarose beads. Exemplary agarose particles include Sepharose™ beads. In some embodiments, cyanogen bromide-activated 4% crosslinked agarose beads having diameters in the range of 40-165 m may be derivatized with polyC-containing initiators for use with methods of the invention. In other embodiments, cyanogen bromide-activated 6% crosslinked agarose beads having diameters in the range of 200-300 m may be used with methods of the invention. In the latter two embodiments, polyC-containing oligonucleotide initiators having a 5′-aminolinker may be coupled to the Sepharose™ beads for use with the invention. Other desirable linkers for agarose beads include thiol and epoxy linkers.

In some embodiments, a porous resin support derivatized with polyC-containing initiators has average pore diameters of at least 10 nm, or at least 20 nm, or at least 50 nm. In other embodiments, such porous resin support has an average pore diameter in the range of from 10 nm to 500 nm, or in the range of from 50 nm to 500 nm.

In some embodiments, polyC-containing initiators are attached to planar supports for massively parallel synthesis of oligonucleotides, e.g. via inkjet delivery of reagents, such as described by Horgan et al, International patent publication WO2020/020608, which is incorporated herein by reference. In some embodiments such planar supports comprise a uniform coating of polyC-containing initiators with protected 3′-hydroxls, wherein, for example, discrete reaction sites may be defined by delivering deprotection solution to discrete locations. In other embodiments, such planar supports comprise an array of discrete reaction sites each containing polyC-containing initiators, which, for example, may be formed on a substrate by photolithographic methods of Brennan, U.S. Pat. No. 5,474,796; Peck et al, U.S. patent Ser. No. 10/384,189; Indermuhle et al, U.S. patent Ser. No. 10/669,304; Fixe et al, Materials Research Society Symposium Proceedings. Volume 723, Molecularly Imprinted Materials—Sensors and Other Devices. Symposia (San Francisco, California on Apr. 2-5, 2002); or like references.

After synthesis is completed polynucleotides with the desired nucleotide sequence may be released from initiators and the solid supports by cleavage. A wide variety of cleavable linkages or cleavable nucleotides may be used for this purpose. In some embodiments, cleaving the desired polynucleotide leaves a natural free 5′-hydroxyl on a cleaved strand; however, in alternative embodiments, a cleaving step may leave a moiety, e.g. a 5′-phosphate, that may be removed in a subsequent step, e.g. by phosphatase treatment. Cleaving steps may be carried out chemically, thermally, enzymatically or by photochemical methods. In some embodiments, cleavable nucleotides may be nucleotide analogs such as deoxyuridine or 8-oxo-deoxyguanosine that are recognized by specific glycosylases (e.g. uracil deoxyglycosylase followed by endonuclease VIII, and 8-oxoguanine DNA glycosylase, respectively). In some embodiments, cleavage may be accomplished by providing initiators with a deoxyinosine as the penultimate 3′ nucleotide, which may be cleaved by endonuclease V at the 3′ end of the initiator leaving a 5′-phosphate on the released polynucleotide. Further methods for cleaving single stranded polynucleotides are disclosed in the following references, which are incorporated by reference: U.S. Pat. Nos. 5,739,386, 5,700,642 and 5,830,655; and U.S. Patent Publication Nos. 2003/0186226 and 2004/0106728; and in Urdea and Horn, U.S. Pat. No. 5,367,066.

In some embodiments, cleavage by glycosylases and/or endonucleases may require a double stranded DNA substrate.

Returning to FIG. 1, in some embodiments, an ordered sequence of nucleotides are coupled to an initiator nucleic acid using a template-free polymerase, such as TdT, in the presence of 3′-O-protected NTPs in each synthesis step. In some embodiments, the method of synthesizing an oligonucleotide comprises the steps of (a) providing an initiator having a free 3′-hydroxyl (100); (b) reacting (104) under extension conditions the initiator or an extension intermediate having a free 3′-hydroxyl with a template-free polymerase in the presence of a 3′-O-protected nucleoside triphosphate to produce a 3′-O-protected extension intermediate (106); (c) deprotecting the extension intermediate to produce an extension intermediate with a free 3′-hydroxyl (108); and (d) repeating steps (b) and (c) (110) until the polynucleotide is synthesized. (Sometimes the terms “extension intermediate” and “elongation fragment” are used interchangeably). In some embodiments, an initiator is provided as an oligonucleotide attached to a solid support, e.g. by its 5′ end. The above method may also include a washing step after each reaction, or extension, step, as well as after each de-protecting step. For example, the step of reacting may include a sub-step of removing unincorporated nucleoside triphosphates, e.g. by washing, after a predetermined incubation period, or reaction time. Such predetermined incubation periods or reaction times typically may be a few seconds, e.g. 30 sec, to several minutes, e.g. 30 min.

When the sequence of polynucleotides on a synthesis support includes reverse complementary subsequences, secondary intra-molecular or cross-molecular structures may be created by the formation of hydrogen bonds between the reverse complementary regions. In some embodiments, base protecting moieties for exocyclic amines are selected so that hydrogens of the protected nitrogen cannot participate in hydrogen bonding, thereby preventing the formation of such secondary structures. That is, base protecting moieties may be employed to prevent the formation of hydrogen bonds, such as are formed in normal base pairing, for example, between nucleosides A and T and between G and C. At the end of a synthesis, the base protecting moieties may be removed and the polynucleotide product may be cleaved from the solid support, for example, by cleaving it from its initiator.

In addition to providing 3′-O-blocked NTP monomers with base protection groups, elongation reactions may be performed at higher temperatures using thermal stable template-free polymerases. For example, a thermal stable template-free polymerase having activity above 40° C. may be employed; or, in some embodiments, a thermal stable template-free polymerase having activity in the range of from 40-85° C. may be employed; or, in some embodiments, a thermal stable template-free polymerase having activity in the range of from 40-65° C. may be employed.

In some embodiments, elongation conditions may include adding solvents to an elongation reaction mixture that inhibit hydrogen bonding or base stacking. Such solvents include water miscible solvents with low dielectric constants, such as dimethyl sulfoxide (DMSO), methanol, and the like. Likewise, in some embodiments, elongation conditions may include the provision of chaotropic agents that include, but are not limited to, n-butanol, ethanol, guanidinium chloride, lithium perchlorate, lithium acetate, magnesium chloride, phenol, 2-propanol, sodium dodecyl sulfate, thiourea, urea, and the like. In some embodiments, elongation conditions include the presence of a secondary-structure-suppressing amount of DMSO. In some embodiments, elongation conditions may include the provision of DNA binding proteins that inhibit the formation of secondary structures, wherein such proteins include, but are not limited to, single-stranded binding proteins, helicases, DNA glycolases, and the like.

3′-O-blocked dNTPs without base protection may be purchased from commercial vendors or synthesized using published techniques, e.g. U.S. Pat. No. 7,057,026; Guo et al, Proc. Natl. Acad. Sci., 105(27): 9145-9150 (2008); Benner, U.S. Pat. Nos. 7,544,794 and 8,212,020; International patent publications WO2004/005667, WO91/06678; Canard et al, Gene (cited herein); Metzker et al, Nucleic Acids Research, 22: 4259-4267 (1994); Meng et al, J. Org. Chem., 14: 3248-3252 (3006); U.S. patent publication 2005/037991. 3′-O-blocked dNTPs with base protection may be synthesized as described below.

When base-protected dNTPs are employed the method of FIG. 1 may further include a step (e) removing base protecting moieties, which in the case of acyl or amidine protection groups may (for example) include treating with concentrated ammonia.

The above method may also include one or more capping steps in addition to washing steps after the reacting, or extending, step A first capping step may cap, or render inert to further extensions, unreacted 3′—OH groups on partially synthesized polynucleotides. Such capping step is usually implemented after a coupling steps, and whenever a capping compound is used, it is selected to be unreactive with protection groups of the monomer just coupled to the growing strands. In some embodiments, such capping steps may be implemented by coupling (for example, by a second enzymatic coupling step) a capping compound that renders the partially synthesized polynucleotide incapable of further couplings, e.g. with TdT. Such capping compounds may be a dideoxynucleoside triphosphate. In other embodiments, non-extended strands with free 3′-hydroxyls may be degraded by treating them with a 3′-exonuclease activity, e.g. Exo I. For example, see Hyman, U.S. Pat. No. 5,436,143. Likewise, in some embodiments, strands that fail to be deblocked may be treated to either remove the strand or render it inert to further extensions. A second capping step may be implemented after a deprotection step, to render the affected strands inert from any subsequent coupling or deprotection any 3′-O protection, or blocking groups. Capping compounds of such second capping step are selected so that they do not react with free 3′-hydroxyls that may be present. In some embodiments, such second capping compound may be a conjugate of an aldehyde group and a hydrophobic group. The latter group permits separation based on hydrophobicity, e.g. Andrus, U.S. Pat. No. 5,047,524.

Exemplary reaction conditions for an elongation step (also sometimes referred to as an extension step or a coupling step) comprise the following: 2.0-20. μM purified TdT; 125-600 μM 3′-O-blocked dNTP (e.g. 3′-O—NH₂-blocked dNTP); about 10 to about 500 mM potassium cacodylate buffer (pH between 6.5 and 7.5) and from about 0.01 to about 10 mM of a divalent cation (e.g. CoCl₂ or MnCl₂), where the elongation reaction may be carried out in a 50 μL reaction volume, at a temperature within the range RT to 45° C., for 3-5 minutes. In embodiments, in which the 3′-O-blocked dNTPs are 3′-O—NH₂-blocked dNTPs, reaction conditions for a deblocking step may comprise the following: 700-1500 mM NaNO₂; 500-1000 mM sodium acetate (adjusted with acetic acid to pH in the range of 4.8-6.5), where the deblocking reaction may be carried out in a 50 μL volume, at a temperature within the range of RT to 45° C. for 30 seconds to several minutes. Washes may be performed with the cacodylate buffer without the components of the coupling reaction (e.g. enzyme, monomer, divalent cations).

Depending on particular applications, the steps of deblocking and/or cleaving may include a variety of chemical or physical conditions, e.g. light, heat, pH, presence of specific reagents, such as enzymes, which are able to cleave a specified chemical bond. Guidance in selecting 3′-O-blocking groups and corresponding de-blocking conditions may be found in the following references, which are incorporated by reference: Benner, U.S. Pat. Nos. 7,544,794 and 8,212,020; 5,808,045; 8,808,988; International patent publication WO91/06678; and references cited below. In some embodiments, the cleaving agent (also sometimes referred to as a de-blocking reagent or agent) is a chemical cleaving agent, such as, for example, dithiothreitol (DTT). In alternative embodiments, a cleaving agent may be an enzymatic cleaving agent, such as, for example, a phosphatase, which may cleave a 3′-phosphate blocking group. It will be understood by the person skilled in the art that the selection of deblocking agent depends on the type of 3′-nucleotide blocking group used, whether one or multiple blocking groups are being used, whether initiators are attached to living cells or organisms or to solid supports, and the like, that necessitate mild treatment. For example, a phosphine, such as tris(2-carboxyethyl)phosphine (TCEP) can be used to cleave a 3′-azidomethyl groups, palladium complexes can be used to cleave a 3′-allyl groups, or sodium nitrite can be used to cleave a 3′O-amino group. In particular embodiments, the cleaving reaction involves TCEP, a palladium complex or sodium nitrite.

As noted above, in some embodiments it is desirable to employ two or more blocking groups that may be removed using orthogonal de-blocking conditions. The following exemplary pairs of blocking groups may be used in parallel synthesis embodiments. It is understood that other blocking group pairs, or groups containing more than two, may be available for use in these embodiments of the invention.

| | |
|---|---|
| 3′-O-NH2 | 3′-O-azidomethyl |
| 3′-O-NH2 | 3′-O-allyl, 3′-O-propargyl |
| 3′-O-NH2 | 3′-O-phosphate |
| 3′-O-azidomethyl | 3′-O-allyl, 3′O-propargyl |
| 3′-O-azidomethyl | 3′-O-phosphate |
| 3′-O-allyl, 3′O-propargyl | 3′-O-phosphate |

Synthesizing oligonucleotides on living cells requires mild deblocking, or deprotection, conditions, that is, conditions that do not disrupt cellular membranes, denature proteins, interfere with key cellular functions, or the like. In some embodiments, deprotection conditions are within a range of physiological conditions compatible with cell survival. In such embodiments, enzymatic deprotection is desirable because it may be carried out under physiological conditions. In some embodiments specific enzymatically removable blocking groups are associated with specific enzymes for their removal. For example, ester- or acyl-based blocking groups may be removed with an esterase, such as acetylesterase, or like enzyme, and a phosphate blocking group may be removed with a 3′ phosphatase, such as T4 polynucleotide kinase. By way of example, 3′-O-phosphates may be removed by treatment with as solution of 100 mM Tris-HCl (pH 6.5) 10 mM MgCl₂, 5 mM 2-mercaptoethanol, and one Unit T4 polynucleotide kinase. The reaction proceeds for one minute at a temperature of 37° C.

In some embodiments, 3′-0 blocking groups include 3′-O-azidomethyl, 3′-O—NH₂, 3′-O-allyl, In some embodiments, blocking group include 3′-O-methyl, 3′-O-(2-nitrobenzyl), 3′-O-allyl, 3′-O-amine, 3′-O-azidomethyl, 3′-O-tert-butoxy ethoxy, 3′-O-(2-cyanoethyl), 3′-O-nitro, and 3′-O-propargyl. In other embodiments, the 3′-blocked nucleotide triphosphate is blocked by either a 3′-O-azidomethyl or a 3′-O—NH₂. Synthesis and use of such 3′-blocked nucleoside triphosphates are disclosed in the following references: U.S. Pat. Nos. 9,410,197; 8,808,988; 6,664,097; 5,744,595; 7,544,794; 8,034,923; 8,212,020; 10472383; Guo et al, Proc. Natl. Acad. Sci., 105(27): 9145-9150 (2008); and like references.

Depending on particular applications, the steps of deblocking and/or cleaving may include a variety of chemical or physical conditions, e.g. light, heat, pH, presence of specific reagents, such as enzymes, which are able to cleave a specified chemical bond. Guidance in selecting 3′-O-blocking groups and corresponding de-blocking conditions may be found in references, such as Wuts, Green's Protection Groups in Organic Chemistry, 5th Edition (Wiley 2014). In some embodiments, the cleaving agent (also sometimes referred to as a de-blocking reagent or agent) is a chemical cleaving agent, such as, for example, dithiothreitol (DTT). In alternative embodiments, a cleaving agent may be an enzymatic cleaving agent, such as, for example, a phosphatase, which may cleave a 3′-phosphate blocking group. It will be understood by the person skilled in the art that the selection of deblocking agent depends on the type of 3′-nucleotide blocking group used, whether one or multiple blocking groups are being used, whether initiators are attached to living cells or organisms or to solid supports, and the like, that necessitate mild treatment. For example, a phosphine, such as tris(2-carboxyethyl)phosphine (TCEP) can be used to cleave a 3′O-azidomethyl group, palladium complexes can be used to cleave 3′O-allyl group and 3′-O-propargyl group, or sodium nitrite can be used to cleave a 3′O-amino group.

### Template-Free Enzymatic Synthesis of RNA

Methods of the invention comprise the enzymatic synthesis of RNA. In some embodiments, such methods comprise the steps described in FIG. 1 using as a template-free polymerase a poly(A) polymerase (PAP) or a poly(U) polymerase. In some embodiments, PAPs and/or PUPs are used to synthesize a polyribonucleic acid using 3′-O-reversibly protected-rNTP precursors, wherein a single PUP or PAP variant may be employed for coupling all ribonucleoside triphosphate monomers, or in alternative embodiments. In some embodiments, different PUPs and PAPs may be employed for coupling different kinds ribonucleoside triphosphate monomers in the synthesis of a particular RNA. Likewise, in other embodiments, PAPs and/or PUPs may be used to synthesize a polydeoxyribonucleic acid using 3′-O-reversibly protected-dNTP precursors, wherein a single PUP or PAP is employed for coupling all deoxyribonucleoside triphosphate (dNTP) monomers, or in an alternative embodiment, wherein different PUP and PAP polymerases may be employed for coupling different kinds of deoxyribonucleoside triphosphate monomers. In some embodiments for RNA synthesis, the same 3′-O-reversible protecting groups described above for deoxyribonucleotides may also be used with ribonucleotide monomers. In some embodiments for RNA synthesis, said 3′-O-blocked nucleoside triphosphate is a 3′-O-azidomethyl-ribonucleoside triphosphate. In some embodiments, methods may employ PAP and/or PUP variants that have been modified by genetic engineering to improve efficiency of coupling 3′-O-blocked-ribonucleoside triphosphates and 3′-O-blocked-2′-deoxyribonucleoside triphosphates to growing polynucleotide chains in a synthesis, for example, as described below.

In some embodiments, the method of synthesizing an oligoribonucleotide of a predetermined sequence comprises the steps of (a) providing an initiator having a free 3′-hydroxyl; (b) reacting under elongation conditions the initiator or an elongation fragment having a free 3′-hydroxyl with a PAP or a PUP in the presence of a 3′-O-blocked ribonucleoside triphosphate to produce a 3′-O-blocked elongation fragement; (c) deblocking the elongation fragment to produce an elongation fragment with a free 3′-hydroxyl; and (d) repeating steps (b) and (c) until the polyribonucleotide of the predetermined sequence is synthesized, wherein the reaction mixture for elongating the initiators or elongated fragments comprise polyC oligonucleotides capable of forming duplexes with the polynucleotide. In some embodiments, initiators each comprise a segment consisting of a polyC oligonucleotide. In other embodiments, a synthesis support is provided having polyC oligonucleotides attached thereto, eventually in addition to initiators. In still other embodiments, polyC oligonucleotides are provided in solution as a component of an elongation reaction mixture for selected elongation cycles in which G4 structure formation has a high likelihood of occurrence.

In some embodiments, as noted above, an initiator is provided as an oligonucleotide attached to a solid support, e.g. by its 5′ end. The above method may also include washing steps after the reaction, or extension, step, as well as after the de-blocking step. For example, the step of reacting may include a sub-step of removing unincorporated ribonucleoside triphosphates, e.g. by washing, after a predetermined incubation period, or reaction time. Such predetermined incubation periods or reaction times may be a few seconds, e.g. 30 sec, to several minutes, e.g. 30 min.

The above method may also include capping step(s) as well as washing steps after the reacting, or extending, step, as well as after the deblocking step. As mentioned above, in some embodiments, capping steps may be included in which non-extended free 3′-hydroxyls are reacted with compounds that prevents any further extensions of the capped strand. In some embodiments, such compound may be a dideoxynucleoside triphosphate. In other embodiments, non-extended strands with free 3′-hydroxyls may be degraded by treating them with a 3′-exoribonuclease activity, e.g. RNase R (Epicentre). Likewise, in some embodiments, strands that fail to be deblocked may be treated to either remove the strand or render it inert to further extensions.

Exemplary reaction conditions for an extension or elongation step using PAP or PUP comprise the following: Reaction conditions 1 (for primer+AM-rATP): 250 uM AM-rATP, 0.1 uM ATT0488-(rA)5, 1 uM PAP, 1× ATP buffer (20 mM Tris-HCl, 0.6 mM MnCl2, 0.02 mM EDTA, 0.1% BSA, 10% glycerol, 100 mM imidazole, pH 7-8), 37 C, 30 min. Reaction condition 2 (for primer+AM-rGTP): 250 uM rGTP, 0.1 uM ATT0488-(rA)5, 1 uM PAP, 1× GTP buffer (0.6 mM MnCl₂, 0.1% BSA, 10 mM imidazole, pH 6), 37 C, 30 min. In the foregoing, “AM-rNTP” refers to 3′-O-azidomethyl-ribonucleoside triphosphate.

### Template-Free Polymerases for Polynucleotide Synthesis

A variety of different template-free polymerases are available for use in methods of the invention. Template-free polymerases include, but are not limited to, polX family polymerases (including DNA polymerases β, γ and μ), poly(A) polymerases (PAPs), poly(U) polymerases (PUPs), DNA polymerase 0, and the like, for example, described in the following references: Ybert et al, International patent publication WO2017/216472; Champion et al, U.S. patent Ser. No. 10/435,676; Champion et al, International patent publication WO2020/099451; Heinisch et al, International patent publication WO2021/018919. In particular, terminal deoxynucleotidyltransferases (TdTs) and variants thereof are useful in template-free DNA synthesis and PAPs and PUPs and variants thereof are useful in template-free RNA synthesis.

In some embodiments, TdT variants are employed with the invention which display increased incorporation activity with respect to 3′-O-modified nucleoside triphosphates. For example, such TdT variants may be produced using techniques described in Champion et al, U.S. patent Ser. No. 10/435,676, which is incorporated herein by reference. In some embodiments, a TdT variant is employed having an amino acid sequence at least 80 percent identical to a TdT having an amino acid sequence of any of SEQ ID NOs 7 through 20, inclusive, and 24 through 39, inclusive, and one or more of the substitutions listed in Table 1, wherein the TdT variant (i) is capable of synthesizing a nucleic acid fragment without a template and (ii) is capable of incorporating a 3′-O-modified nucleotide onto a free 3′-hydroxyl of a nucleic acid fragment. In some embodiments, the above TdT variants include a substitution at every position listed in Table 1. In some embodiments, the above percent identity value is at least 85 percent identity with the indicated SEQ ID NOs; in some embodiments, the above percent identity value is at least 90 percent identity with the indicated SEQ ID NOs; in some embodiments, the above percent identity value is at least 95 percent identity with the indicated SEQ ID NOs; in some embodiments, the above percent identity value is at least 97 percent identity; in some embodiments, the above percent identity value is at least 98 percent identity; in some embodiments, the above percent identity value is at least 99 percent identity. As used herein, the percent identity values used to compare a reference sequence to a variant sequence do not include the expressly specified amino acid positions containing substitutions of the variant sequence; that is, the percent identity relationship is between sequences of a reference protein and sequences of a variant protein outside of the expressly specified positions containing substitutions in the variant. Thus, for example, if the reference sequence and the variant sequence each comprised 100 amino acids and the variant sequence had mutations at positions 25 and 81, then the percent homology would be in regard to sequences 1-24, 26-80 and 82-100.

TABLE 1

| SEQ | | | | | | |
|---|---|---|---|---|---|---|
| ID NO | Animal | Substitutions | | | | |
| 1 | Mouse | M192R/Q | C302G/R | R336L/N | R454P/N/A/V | E457N/L/T/S/K |
| 7 | Mouse | M63R/Q | C173G/R | R207L/N | R325P/N/A/V | E328N/L/T/S/K |
| 8 | Bovine | M63R/Q | C173G/R | R207L/N | R324P/N/A/V | E327N/L/T/S/K |
| 9 | Human | M63R/Q | C173G/R | R207L/N | R324P/N/A/V | E327N/L/T/S/K |
| 10 | Chicken | — | C172G/R | R206L/N | R320P/N/A/V | — |
| 11 | Possum | M63R/Q | C173G/R | R207L/N | R331P/N/A/V | E334N/L/T/S/K |
| 12 | Shrew | M63R/Q | C173G/R | R207L/N | — | E328N/L/T/S/K |
| 13 | Python | — | C174G/R | R208L/N | R331P/N/A/V | E334N/L/T/S/K |
| 14 | Canine | M73R/Q | C173G/R | R207L/N | R325P/N/A/V | E328N/L/T/S/K |
| 15 | Mole | M64R/Q | C174G/R | R208L/N | — | E329N/L/T/S/K |
| 16 | Pika | M61R/Q | C171G/R | R205L/N | R323P/N/A/V | E326N/L/T/S/K |
| 17 | Hedgehog | M63R/Q | C173G/R | R207L/N | R328P/N/A/V | E331N/L/T/S/K |
| 18 | Tree shrew | — | C173G/R | R207L/N | R325P/N/A/V | E328N/L/T/S/K |
| 19 | Platypus | M63R/Q | C182G/R | R216L/N | R338P/N/A/V | E341N/L/T/S/K |
| 20 | Jerboa | M66R/Q | C176G/R | R210L/N | R328P/N/A/V | E331N/L/T/S/K |
| 24 | Canary | — | C170G/R | R204L/N | R326P/N/A/V | E329N/L/T/S/K |
| 25 | Neopelma | — | C158G/R | R192L/N | R314P/N/A/V | E317N/L/T/S/K |
| 26 | Alligator | — | — | R205L/N | R327P/N/A/V | E330N/L/T/S/K |
| 27 | Xenopus | — | — | R205L/N | R324P/N/A/V | E327N/L/T/S/K |
| 28 | Tiger snake | — | — | R205L/N | R327P/N/A/V | E330N/L/T/S/K |
| 29 | Brown trout | — | — | R192L/N | R311P/N/A/V | E314N/L/T/S/K |
| 30 | Electric eel | — | — | R205L/N | R321P/N/A/V | E325N/L/T/S/K |
| 31 | Walking fish | — | — | R205L/N | R322P/N/A/V | E325N/L/T/S/K |
| 32 | Guppy | — | — | R205L/N | R322P/N/A/V | E325N/L/T/S/K |
| 33 | Rat | M48R/Q | C158G/R | R192L/N | R310P/N/A/V | E313N/L/T/S/K |
| 34 | Rat | M61R/Q | C171G/R | R205L/N | R323P/N/A/V | E326N/L/T/S/K |
| 35 | Colobus monkey | M61R/Q | C171G/R | R205L/N | R323P/N/A/V | E326N/L/T/S/K |
| 36 | Pig | M61R/Q | C171G/R | R205L/N | R323P/N/A/V | E326N/L/T/S/K |
| 37 | Tiger | M61R/Q | C171G/R | R205L/N | R323P/N/A/V | E326N/L/T/S/K |
| 38 | Water buffalo | M48R/Q | C158G/R | R192L/N | R310P/N/A/V | E313N/L/T/S/K |
| 39 | Marmot | M61R/Q | C171G/R | R205L/N | R323P/N/A/V | E326N/L/T/S/K |

In some embodiments, a TdT variant of the invention is derived from a TdT comprising an amino acid sequence at least 80 percent identical to an amino acid sequence selected from SEQ TD NOs 40 through 75, inclusive, and one or more of the substitutions listed in Table 1, wherein the TdT variant (i) is capable of synthesizing a nucleic acid fragment without a template and (ii) is capable of incorporating a 3′-O-modified nucleotide onto a free 3′-hydroxyl of a nucleic acid fragment. In some embodiments, the above TdT variants include a substitution at every position listed in Table 2. In some embodiments, the above percent identity value is at least 85 percent identity with the indicated SEQ TD NOs; in some embodiments, the above percent identity value is at least 90 percent identity with the indicated SEQ ID NOs; in some embodiments, the above percent identity value is at least 95 percent identity with the indicated SEQ ID NOs; in some embodiments, the above percent identity value is at least 97 percent identity; in some embodiments, the above percent identity value is at least 98 percent identity; in some embodiments, the above percent identity value is at least 99 percent identity. As above, the percent identity values used to compare a reference sequence to a variant sequence do not include the expressly specified amino acid positions containing substitutions of the variant sequence; that is, the percent identity relationship is between sequences of a reference protein and sequences of a variant protein outside of the expressly specified positions containing substitutions in the variant.

TdT variants of SEQ ID NOs 40 through 54, inclusive, 56, 59, 61, 63, 65, 67, 69, 70, 73 and 74 includes substitutions at one or more of the indicated amino acid positions as listed in Table 2 in addition to a stabilizing substitution of the glutamine at position 4 (or a functionally equivalent position). In other embodiments, TdT variants of the invention are derived from natural TdTs such as those listed in Table 2 with a substitution at every one of the indicated amino acid positions in addition to the stabilizing substitution of the glutamine at position 4. In some embodiments, such stabilizing amino acid substituted for glutamine is selected from the group consisting of E, S, D and N. In other embodiments, the stabilizing amino acid is E.

TABLE 2

| SEQ | | | | | | |
|---|---|---|---|---|---|---|
| ID NO | Animal | Substitutions | | | | |
| 1 | Mouse | M192R/Q | C302G/R | R336L/N | R454P/N/A/V | E457N/L/T/S/K |
| 40 | Mouse | M44R/Q | C154G/R | R188L/N | R306P/N/A/V | E309N/L/T/S/K |
| 41 | Bovine | M44R/Q | C154G/R | R188L/N | R305P/N/A/V | E308N/L/T/S/K |
| 42 | Human | M44R/Q | C154G/R | R188L/N | R305P/N/A/V | E308N/L/T/S/K |
| 43 | Chicken | — | C154G/R | R188L/N | R302P/N/A/V | — |
| 44 | Possum | M44R/Q | C154G/R | R188L/N | R312P/N/A/V | E315N/L/T/S/K |
| 45 | Shrew | M44R/Q | C154G/R | R188L/N | — | E309N/L/T/S/K |
| 46 | Canine | M44R/Q | C154G/R | R188L/N | R306P/N/A/V | E309N/L/T/S/K |
| 47 | Mole | M44R/Q | C154G/R | R188L/N | — | E309N/L/T/S/K |
| 48 | Pika | M44R/Q | C154G/R | R188L/N | R306P/N/A/V | E309N/L/T/S/K |
| 49 | Hedgehog | M44R/Q | C154G/R | R188L/N | R309P/N/A/V | E312N/L/T/S/K |
| 50 | Tree shrew | — | C154G/R | R188L/N | R306P/N/A/V | E309N/L/T/S/K |
| 51 | Platypus | M44R/Q | C163G/R | R197L/N | R319P/N/A/V | E322N/L/T/S/K |
| 52 | Canary | — | C153G/R | R187L/N | R309P/N/A/V | — |
| 53 | Neopelma | — | C154G/R | R188L/N | R310P/N/A/V | E311N/L/T/S/K |
| 54 | Alligator | — | — | R188L/N | R310P/N/A/V | E313N/L/T/S/K |
| 56 | Xenopus | — | — | R188L/N | R307P/N/A/V | E310N/L/T/S/K |
| 59 | Brown Trout | — | — | R188L/N | — | E310N/L/T/S/K |
| 61 | Electric eel | — | — | R188L/N | — | — |
| 63 | Walking fish | — | — | R188L/N | R305P/N/A/V | E308N/L/T/S/K |
| 65 | Guppy | — | — | R188L/N | R305P/N/A/V | E308N/L/T/S/K |
| 67 | Rat | — | — | R188L/N | R306P/N/A/V | E309N/L/T/S/K |
| 69 | Piliocolobus | — | — | R188L/N | R306P/N/A/V | E309N/L/T/S/K |
| 70 | Pig | M44R/Q | C154G/R | R188L/N | R306P/N/A/V | E309N/L/T/S/K |
| 73 | Water buffalo | M44R/Q | C154G/R | R188L/N | R305P/N/A/V | E308N/L/T/S/K |
| 74 | Marmot | M44R/Q | C154G/R | R188L/N | R306P/N/A/V | E309N/L/T/S/K |

In some embodiments, further TdT variants for use with methods of the invention include one or more of the substitutions of methionine, cysteine, arginine (first position), arginine (second position) or glutamic acid, as shown in Table 2.

In some embodiments, a TdT variant comprising an amino acid sequence at least ninety percent identical to an amino acid sequence of SEQ ID NOs 55, 57, 58, 60, 62, 64, 66, 68, 71, 72, and 75 through 112, inclusive, may also be used with the present invention.

In regard to TdT variants of SEQ ID NOs 7 through 112, in some embodiments, a 3′-O-modified nucleotide may comprise a 3′-O—NH₂-nucleoside triphosphate, a 3′-O-azidomethyl-nucleoside triphosphate, a 3′-O-allyl-nucleoside triphosphate, a 3′O-(2-nitrobenzyl)-nucleoside triphosphate, or a 3′-O-propargyl-nucleoside triphosphate.

A wide variety of PAPs may be used with the method of the invention, including PAP variants that have been engineered for improved characteristics, such as, higher incorporation rates of 3′-O-protected-rNTPs (including for particular protection groups, such as, 3′-O-azidomethyl), greater stability and shelf life, thermostability, solubility, and the like. In particular, a yeast PAP with a mutation at M310 (SEQ ID NO: 1), or a functionally equivalent residue in other PAPs, such as PAPs from various different species, shows improved incorporation of 3′-O-protected rNTPs with respect to a wildtype PAP. In some embodiments, a yeast PAP variant of the invention has an amino acid sequence of SEQ ID NO: 1 except for a substitution at M310. In some embodiments, such substitution is selected from M310F/Y/V/E/T. In particular, substitutions M310F/Y allow the incorporation of 3′-O-amino-rATPs and substitutions M310V/E/T improve the rate of incorporation of 3′-O-protected-rGTPs. In other embodiments, a yeast PAP variant of the invention has an amino acid sequence with at least 90 percent identity of SEQ ID NO: 1 except for a substitution at M310.

PAP variants for use with the invention include those listed in Table 3 below. In some embodiments PAP variants of the invention comprise at least a substitution at the second position indicated in Table 3. In other embodiments, embodiments of PAP variants of the invention comprise at least a substitution at the first position indicated in Table 3.

TABLE 3
PAP Variants: Positions of Substitutions

| SEQ ID NO | Organism | First Position | Second Position |
|---|---|---|---|
| 113 | yeast | V234 | M310 |
| 114 | Myceliophthora | V240 | M318 |
| 115 | Thielavia | V240 | M318 |
| 116 | Pyronema | I237 | M316 |
| 117 | Tilletia | V232 | M309 |
| 118 | Clathrospora | V240 | M316 |
| 119 | Drechslerella | V196 | M272 |
| 120 | Magnaporthiopsis | V240 | M316 |
| 121 | Cryptococcus | V229 | M307 |
| 122 | Golovinomyces | V236 | M313 |
| 123 | Hortaea | V236 | M312 |
| 124 | Valsa | V241 | M317 |
| 125 | Wallemia | V233 | M316 |
| 126 | Xylaria | V240 | M316 |
| 127 | Chaetomium | V240 | M312 |
| 128 | Lachancea | V234 | M310 |
| 129 | Schizosaccharomyces | V233 | M309 |
| 130 | Exophiala | V237 | M317 |
| 131 | Scedosporium | V238 | M314 |
| 132 | Trichoderma | V231 | M307 |
| 133 | Aspergillus | V239 | M315 |
| 134 | Sodiomyces | V240 | M316 |
| 135 | Neohortaea | V235 | M311 |

In some embodiments, a substitution at a first position as indicated in Table 3 is A or G (thus, for example, for SEQ ID NO:113, the substitution may be written V234A/G). In some embodiments, a substitution at a second position as indicated in Table 3 is F, Y, V, E, or T (thus, for example, for SEQ ID NO: 113, the substitution may be written M310F/Y/V/E/T)

In some embodiments, a PAP variant of the invention has one or more of the substitutions of Table 3 and a percent identity value of at least 80 percent identity with the indicated SEQ ID NO; in some embodiments, the above percent identity value is at least 90 percent identity with the indicated SEQ ID NO; in some embodiments, the above percent identity value is at least 95 percent identity with the indicated SEQ ID NO; in some embodiments, the above percent identity value is at least 97 percent identity; in some embodiments, the above percent identity value is at least 98 percent identity; in some embodiments, the above percent identity value is at least 99 percent identity.

In some embodiments, a thermostable PAP is employed so that the method may be practiced at a temperature that reduces or eliminates the formation of secondary structures in the RNA or DNA being synthesized. In some embodiments, the temperature range within which the highest incorporation rate occurs for the thermostable PAP is higher than 40° C. In some embodiments, the temperature range within which the highest incorporation rate occurs for the thermostable PAP is higher than 50° C. In some embodiments, the temperature range within which the highest incorporation rate occurs for the thermostable PAP is between 40° C. and 85° C. In some embodiments, the temperature range within which the highest incorporation rate occurs for the thermostable PAP is between 50° C. and 85° C.

As with PAPs, a wide variety of PUPs may be used with the method of the invention, including PUP variants that have been engineered for improved characteristics, such as, higher incorporation rates of 3′-O-protected-rNTPs (including for particular protection groups, such as, 3′-O-azidomethyl), greater stability and shelf life, thermostability, solubility, and the like. PUP variants for use with the invention include those listed in Table 4 below. In some embodiments PUP variants of the invention comprise at least a substitution at the first position indicated in Table 4. In other embodiments, embodiments of PUP variants of the invention comprise at least a substitution at the second position indicated in Table 4.

TABLE 4
PUP Variants: Positions of Substitutions

| SEQ ID NO | Organism | First Position | Second Position |
|---|---|---|---|
| 136 | S. pombe | Y212 | H336 |
| 137 | T. brucei | Y189 | L303 |
| 138 | S. pombe | Y184 | H308 |
| 139 | T. boudieri | Y227 | H364 |
| 140 | D. stenobrocha | Y478 | H613 |
| 141 | Phytomonas | Y192 | L306 |
| 142 | B. saltans | Y186 | L326 |
| 143 | A. deanei | Y243 | L392 |
| 144 | P. lactucaedebilis | Y196 | H330 |
| 145 | S. culicis | Y253 | L392 |
| 146 | B. meristosporus | Y284 | H408 |
| 147 | N. californiae | Y182 | H310 |
| 148 | Perkinsela | Y187 | L394 |
| 149 | S. complicate | Y203 | H331 |
| 150 | S. ochraceum | Y224 | F349 |
| 151 | G. androsaceus | Y204 | Y332 |
| 152 | T. equiperdum | Y337 | L473 |
| 153 | M. conica | Y296 | H431 |
| 154 | P. murina | Y291 | H423 |
| 155 | S. japonicus | Y218 | H340 |
| 156 | A. nigricans | Y366 | H509 |

In some embodiments, a substitution at a first position as indicated in Table 4 is A or G (thus, for example, for SEQ ID NO: 136, the substitution may be written Y212A/G). In some embodiments, a substitution at a second position as indicated in Table 4 is F, Y, V, E, or T (thus, for example, for SEQ ID NO: 4, the substitution may be written H336F/Y/V/E/T)

In some embodiments, a PUP variant of the invention has one or more of the substitutions of Table 4 and a percent identity value of at least 80 percent identity with the indicated SEQ ID NO; in some embodiments, the above percent identity value is at least 90 percent identity with the indicated SEQ ID NO; in some embodiments, the above percent identity value is at least 95 percent identity with the indicated SEQ ID NO; in some embodiments, the above percent identity value is at least 97 percent identity; in some embodiments, the above percent identity value is at least 98 percent identity; in some embodiments, the above percent identity value is at least 99 percent identity.

In some embodiments, a thermostable PUP is employed so that the method may be practiced at a temperature that reduces or eliminates the formation of secondary structures in the RNA or DNA being synthesized. In some embodiments, the temperature range within which the highest incorporation rate occurs for the thermostable PUP is higher than 40° C. In some embodiments, the temperature range within which the highest incorporation rate occurs for the thermostable PUP is higher than 50° C. In some embodiments, the temperature range within which the highest incorporation rate occurs for the thermostable PUP is between 40° C. and 85° C. In some embodiments, the temperature range within which the highest incorporation rate occurs for the thermostable PUP is between 50° C. and 85° C.

TdT, PAP and PUP variants for use with the invention each comprise an amino acid sequence having a percent sequence identity with a specified SEQ ID NO, subject to the presence of indicated substitutions. In some embodiments, the number and type of sequence differences between a variant of the invention described in this manner and the specified SEQ ID NO may be due to substitutions, deletion and/or insertions, and the amino acids substituted, deleted and/or inserted may comprise any amino acid. In some embodiments, such deletions, substitutions and/or insertions comprise only naturally occurring amino acids. In some embodiments, substitutions comprise only conservative, or synonymous, amino acid changes, as described in Grantham, Science, 185: 862-864 (1974). That is, a substitution of an amino acid can occur only among members of its set of synonymous amino acids. In some embodiments, sets of synonymous amino acids that may be employed are set forth in Table 5A.

TABLE 5A
Synonymous Sets of Amino Acids I

| Amino Acid | Synonymous Set |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Gly, Ala, Thr, Pro, Ser |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Cys, Ser, Thr |
| His | His, Glu, Lys, Gln, Thr, Arg |
| Gln | Gln, Glu, Lys, Asn, His, Thr, Arg |
| Asn | Asn, Gln, Asp, Ser |
| Lys | Lys, Glu, Gln, His, Arg |
| Asp | Asp, Glu, Asn |
| Glu | Glu, Asp, Lys, Asn, Gln, His, Arg |
| Met | Met, Phe, Ile, Val, Leu |
| Trp | Trp |

In some embodiments, sets of synonymous amino acids that may be employed are set forth in Table 5B.

TABLE 5B
Synonymous Sets of Amino Acids II

| Amino Acid | Synonymous Set |
|---|---|
| Ser | Ser |
| Arg | Arg, Lys, His |
| Leu | Ile, Phe, Met, Leu |
| Pro | Ala, Pro |
| Thr | Thr |
| Ala | Pro, Ala |
| Val | Met, Ile Val |
| Gly | Gly |
| Ile | Met, Phe, Val, Leu, Ile |
| Phe | Met, Tyr, Ile, Leu, Phe |
| Tyr | Trp, Met |
| Cys | Cys, Ser |
| His | His, Gln, Arg |
| Gln | Gln, Glu, His |
| Asn | Asn, Asp |
| Lys | Lys, Arg |
| Asp | Asp, Asn |
| Glu | Glu, Gln |
| Met | Met, Phe, Ile, Val, Leu |
| Trp | Trp |

TdT, PAP and PUP variants for use with the invention are produced by conventional biotechnology technics and may include an affinity tag for purification, which may be attached to the N-terminus, C-terminus or at an interior position of the template-free polymerase. In some embodiments, affinity tags are cleaved before the template-free polymerase is used. In other embodiments, affinity tags are not cleaved before use. In some embodiments, a peptide affinity tag is inserted into a loop 2 region of a TdT variant. An exemplary N-terminal His-tag for use with TdT variants of the invention is MASSHHHHHHSSGSENLYFQTGSSG- (SEQ ID NO: 6)). Guidance for selecting a peptide affinity tag is described in the following references: Terpe, Appl. Microbiol. Biotechnol., 60: 523-533 (2003); Arnau et al, Protein Expression and Purification, 48: 1-13 (2006); Kimple et al, Curr. Protoc. Protein Sci., 73: Unit-9.9 (2015); Kimple et al, U.S. Pat. No. 7,309,575; Lichty et al, Protein Expression and Purification, 41: 98-105 (2005); and the like. Guidance for selecting a peptide affinity tag is described in the following references: Terpe, Appl. Microbiol. Biotechnol., 60: 523-533 (2003); Arnau et al, Protein Expression and Purification, 48: 1-13 (2006); Kimple et al, Curr. Protoc. Protein Sci., 73: Unit-9.9 (2015); Kimple et al, U.S. Pat. No. 7,309,575; Lichty et al, Protein Expression and Purification, 41: 98-105 (2005); and the like.

### Measurement of Nucleotide Incorporation Activity

The efficiency of nucleotide incorporation by variants used with the invention may be measured by an extension, or elongation, assay, e.g. as described in Boule et al (cited below); Bentolila et al (cited below); and Hiatt et al, U.S. Pat. No. 5,808,045, the latter of which is incorporated herein by reference. Briefly, in one form of such an assay, a fluorescently labeled oligonucleotide having a free 3′-hydroxyl is reacted with a template-free polymerase, such as a TdT, under extension conditions for a predetermined duration in the presence of a reversibly blocked nucleoside triphosphate, after which the extension reaction is stopped and the amounts of extension products and unextended oligonucleotide are quantified after separation by gel electrophoresis. By such assays, the incorporation efficiency of a variant template-free polymerase may be readily compared to the efficiencies of other variants or to that of wild type or reference polymerases. In some embodiments, a measure of template-free polymerase efficiency may be a ratio (given as a percentage) of amount of extended product using the variant template-free polymerase over the amount of extended product using wild type template-free polymerase, or reference polymerase, in an equivalent assay.

In some embodiments, the following particular extension assay may be used to measure incorporation efficiencies of TdTs: The primer used is the following:

(SEQ ID NO: 5)
5′-AAAAAAAAAAAAAAGGGG-3′

The primer has also an ATTO fluorescent dye on the 5′ extremity. Representative modified nucleotides used (noted as dNTP in Table 6) include 3′-O-amino-2′,3′-dideoxynucleotides-5′-triphosphates (—ONH₂, Firebird Biosciences), such as 3′-O-amino-2′,3′-dideoxyadenosine-5′-triphosphate. For each different variant tested, one tube is used for the reaction. The reagents are added to the tube, starting from water, and then in the order of Table 6. After 30 min at 37° C. the reaction is stopped by addition of formamide (Sigma).

TABLE 6
Extension Activity Assay Reagents

| | | |
|---|---|---|
| Reagent | Concentration | Volume |

| | | |
|---|---|---|
| H₂O | — | 12 μL |
| Activity buffer | 10x | 2 μL |

| | | | |
|---|---|---|---|
| dNTP | 250 | μM | 2 μL |
| Purified enzyme | 20 | μM | 2 μL |
| Fluorescent primer | 500 | nM | 2 μL |

The Activity buffer comprises, for example, TdT reaction buffer (available from New England Biolabs) supplemented with CoCl₂.

The product of the assay is analyzed by conventional polyacrylamide gel electrophoresis. For example, products of the above assay may be analyzed in a 16 percent polyacrylamide denaturing gel (Bio-Rad). Gels are made just before the analysis by pouring polyacrylamide inside glass plates and let it polymerize. The gel inside the glass plates is mounted on an adapted tank filed with TBE buffer (Sigma) for the electrophoresis step. The samples to be analyzed are loaded on the top of the gel. A voltage of 500 to 2,000V is applied between the top and bottom of the gel for 3 to 6 h at room temperature. After separation, gel fluorescence is scanned using, for example, a Typhoon scanner (GE Life Sciences). The gel image is analyzed using ImageJ software (imagej.nih.gov/ij/), or its equivalent, to calculate the percentage of incorporation of the modified nucleotides.

The elongation efficiency of a template-free polymerase may also be measured in the following hairpin completion assay. In such assay, a test polynucleotide is provided with a free 3′ hydroxyl such that under reaction conditions it is substantially only single stranded, but that upon extension with a polymerase, such as a TdT variant, it forms a stable hairpin structure comprising a single stranded loop and a double stranded stem. This allows the detection of an extension of the 3′ end by the presence of the double stranded polynucleotide. The double stranded structure may be detected in a variety of ways including, but not limited to, (i) fluorescent dyes that preferentially fluoresce upon intercalation into the double stranded structure, (ii) fluorescent resonance energy transfer (FRET) between an acceptor (or donor) on the extended polynucleotide and a donor (or acceptor) on an oligonucleotide that forms a triplex with the newly formed hairpin stem, (iii) FRET acceptors and donors that are both attached to the test polynucleotide and that are brought into FRET proximity upon formation of a hairpin, or the like. In some embodiments, a stem portion of a test polynucleotide after extension by a single nucleotide is in the range of 4 to 6 basepairs in length; in other embodiments, such stem portion is 4 to 5 basepairs in length; and in still other embodiments, such stem portion is 4 basepairs in length. In some embodiments, a test polynucleotide has a length in the range of from 10 to 20 nucleotides; in other embodiments, a test polynucleotide has a length in the range of from 12 to 15 nucleotides. In some embodiments, it is advantageous or convenient to extend the test polynucleotide with a nucleotide that maximizes the difference between the melting temperatures of the stem without extension and the stem with extension; thus, in some embodiments, a test polynucleotide is extended with a dC or dG (and accordingly the test polynucleotide is selected to have an appropriate complementary nucleotide for stem formation).

Exemplary test polynucleotides for hairpin completion assays include p875 (5′-CAGTTAAAAACT) (SEQ ID NO: 2) which is completed by extending with a dGTP; p876 (5′-GAGTTAAAACT) (SEQ ID NO: 3) which is completed by extending with a dCTP; and p877 (5′-CAGCAAGGCT) (SEQ ID NO: 4) which is completed by extending with a dGTP. Exemplary reaction conditions for such test polynucleotides may comprise: 2.5-5 μM of test polynucleotide, 1:4000 dilution of GelRed® (intercalating dye from Biotium, Inc., Fremont, CA), 200 mM Cacodylate KOH pH 6.8, 1 mM CoCl₂, 0-20% of DMSO and 3′-ONH2 dGTP and TdT at desired concentrations. Completion of the hairpin may be monitored by an increase in fluorescence of GelRed® dye using a conventional fluorimeter, such as a TECAN reader at a reaction temperature of 28-38° C., using an excitation filter set to 360 nm and an emission filter set to 635 nm.

### Kits

The invention includes a variety of kits for practicing methods of the invention. In one aspect, kits of the invention comprise a synthesis support having attached thereto, eventually by a 5′ end, an initiator comprising a polyC oligonucleotide. In some embodiments, such synthesis support is a solid support. In further embodiments, such solid support may comprise particles, which may be porous particles or nonporous particles. Nonporous particles may, for example, comprise magnetic beads. In other embodiments, such particles may comprise porous particles, such as resins or gels. In some embodiments, such resins comprise an agarose resin. In some of the above embodiments, initiators attached to a solid support each comprise one or more polyC oligonucleotides each with length in the range of from 2 to 30 nucleotides. In some embodiments, such solid support is a population of microparticles, especially nonporous microparticles. In other embodiments, such solid support is a population of porous microparticles. In some embodiments such porous microparticles are agarose microparticles. In some embodiments, such solid support is a planar support, such as a glass slide. In some embodiments, such planar support has a uniform coating of initiators containing one or more polyC oligonucleotides. In other embodiments, such planar support has an array of discrete reaction sites each comprising a coating of initiators containing one or more polyC oligonucleotides. In some embodiments, kits of the invention further include one or more template-free polymerase variants in a formulation, or in formulations, if provided separately, suitable for carrying out template-free enzymatic polynucleotide synthesis as described herein. Such kits may also include synthesis buffers for each template-free polymerase variant that provide reaction conditions for optimizing the template-free addition or incorporation of a 3′-O-protected dNTP to a growing strand. In embodiments for synthesizing DNA, a template-free polymerase is a TdT variant. In embodiments for synthesizing RNA, a template-free polymerase is a PAP and/or PUP variant.

In some embodiments, kits of the invention may comprise a solid support having attached thereto by a 5′ end an initiator comprising a polyC oligonucleotide and separate polyC oligonucleotides attached to the same solid support. In additional embodiments, such kits may comprise polyC oligonucleotides in a solution.

In some embodiments, kits of the invention further include 3′-O-reversibly protected dNTPs. In such embodiments, the 3′-O-reversibly protected dNTPs may comprise 3′-O-amino-dNTPs or 3′-O-azidomethyl-dNTPs. In further embodiments, kits may include one or more of the following items, either separately or together with the above-mentioned items: (i) deprotection or de-blocking reagents for carrying out a deprotecting or deblocking step as described herein, (ii) solid supports with initiators attached thereto, (iii) cleavage reagents for releasing completed polynucleotides from solid supports, (iv) wash reagents or buffers for removing unreacted 3′-O-reversibly protected dNTPs at the end of an enzymatic addition or coupling step, and (v) post-synthesis processing reagents, such as purification columns, desalting reagents, eluting reagents, and the like.

In regard to items (ii) and (iii) above, certain initiators and cleavage reagents go together. For example, an initiator comprising an inosine cleavable nucleotide may come with an endonuclease V cleavage reagent; an initiator comprising a nitrobenzyl photocleavable linker may come with a suitable light source for cleaving the photocleavable linker; an initiator comprising a uracil may come with a uracil DNA glycosylase cleavage reagent; and the like.

### Example

### Synthesis of G4-Forming Polynucleotides

### With and Without PolyC Initiators

In this example, eight polydexoxyribonucleotides having G4-prone sequences are synthesized with and without PolyC initiators substantially following the exemplary synthesis protocol described above. Solid supports are CNBr-activated 45 μm agarose beads having either polyC initiator (-CCCCCCCCCCCCCCCTdIT-3′ (SEQ ID NO: 157)) attached via a C15 linker or non-polyC initiator (-TTTTTTTTTTdIT-3′ (SEQ ID NO: 158)) attached via a C15 linker. The template-free polymerase is TdT variant M77 (SEQ ID NO: 106) having N-terminal affinity tag (SEQ ID NO: 6). After synthesis, polynucleotide product is cleaved from the solid supports using EndoV endonuclease, following the protocol described in Creton, International patent publication WO/2020/165137. Cleaved synthesis products are analyzed by capillary electrophoresis to determine the purity of the desired polynucleotide and samples of product are sequenced to assess deletion, substitution and insertion errors. By both measures the use of polyC-containing initiators showed significantly improved yields of the desired products. The purity data indicates that use of the polyC initiator increased the purity of the G4-prone polynucleotide products by an average percentage of 20 percent or greater. The following tables compare error rates of various types in the sequences sampled from the polynucleotides synthesized with and without polyC-containing initiators.

### Error Rates in Average Percentages Using Non-PolyC Initiators

| | A | C | G | T |
|---|---|---|---|---|
| Substitutions | 0.08 | 0.66 | 0.17 | 0.04 |
| Insertions | 0.16 | 0.10 | 0.13 | 0.17 |
| Deletions | 1.11 | 0.62 | 0.64 | 0.52 |

### Error Rates in Average Percentages Using PolyC Initiators

| | A | C | G | T |
|---|---|---|---|---|
| Substitutions | 0.08 | 0.21 | 0.09 | 0.03 |
| Insertions | 0.06 | 0.05 | 0.09 | 0.08 |
| Deletions | 0.94 | 0.28 | 0.37 | 0.30 |

### Definitions

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999).

“Functionally equivalent” in reference to amino acid positions in two or more different TdTs means (i) the amino acids at the respective positions play the same functional role in an activity of the TdTs, and (ii) the amino acids occur at homologous amino acid positions in the amino acid sequences of the respective TdTs. It is possible to identify positionally equivalent or homologous amino acid residues in the amino acid sequences of two or more different TdTs on the basis of sequence alignment and/or molecular modelling. In some embodiments, functionally equivalent amino acid positions belong to inefficiency motifs that are conserved among the amino acid sequences of TdTs of evolutionarily related species, e.g. genus, families, or the like. Examples of such conserved inefficiency motifs are described in Motea et al, Biochim. Biophys. Acta. 1804(5): 1151-1166 (2010); Delarue et al, EMBO J., 21: 427-439 (2002); and like references.

“Kit” refers to any delivery system, such as a package, for delivering materials or reagents for carrying out a method implemented by a system or apparatus of the invention. In some embodiments, consumables materials or reagents are delivered to a user of a system or apparatus of the invention in a package referred to herein as a “kit.” In the context of the invention, such delivery systems include, usually packaging methods and materials that allow for the storage, transport, or delivery of materials, such as, synthesis supports, oligonucleotides, 3′-O-protected-dNTPs, and the like. For example, kits may include one or more enclosures (e.g., boxes) containing solid supports with polyC initiators attached and/or supporting materials. Such contents may be delivered to the intended recipient together or separately. For example, a first container may contain solid supports with polyC initiators attached, while a second or more containers contain a 3′-O-protected-deoxynucleoside triphosphates, a template-free polymerase, for example, a specific TdT variant, and appropriate buffers.

“Mutant” or “variant,” which are used interchangeably, refer to polypeptides derived from a natural or reference TdT polypeptide described herein, and comprising a modification or an alteration, i.e., a substitution, insertion, and/or deletion, at one or more positions. Variants may be obtained by various techniques well known in the art. In particular, examples of techniques for altering the DNA sequence encoding the wild-type protein, include, but are not limited to, site-directed mutagenesis, random mutagenesis, sequence shuffling and synthetic oligonucleotide construction. Mutagenesis activities consist in deleting, inserting or substituting one or several amino-acids in the sequence of a protein or in the case of the invention of a polymerase. The following terminology is used to designate a substitution: L238A denotes that amino acid residue (Leucine, L) at position 238 of a reference, or wild type, sequence is changed to an Alanine (A). A132V/I/M denotes that amino acid residue (Alanine, A) at position 132 of the parent sequence is substituted by one of the following amino acids: Valine (V), Isoleucine (I), or Methionine (M). The substitution can be a conservative or non-conservative substitution. Examples of conservative substitutions are within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

“Polynucleotide” or “oligonucleotide” are used interchangeably and each mean a linear polymer of nucleotide monomers or analogs thereof. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moieties, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references. Likewise, the oligonucleotide and polynucleotide may refer to either a single stranded form or a double stranded form (i.e. duplexes of an oligonucleotide or polynucleotide and its respective complement). It will be clear to one of ordinary skill which form or whether both forms are intended from the context of the terms usage.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2nd Edition (Cold Spring Harbor Press, New York, 2003).

“Sequence identity” refers to the number (or fraction, usually expressed as a percentage) of matches (e.g., identical amino acid residues) between two sequences, such as two polypeptide sequences or two polynucleotide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithm (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://blast.ncbi.nlm.nih.gov/or ttp://www.ebi.ac.uk/Tools/emboss/. Those skilled in the art can determine appropriate parameters for measuring alignment, including any algorithm needed to achieve maximal alignment over the full length of the sequences being compared. For purposes herein, % amino acid sequence identity values refer to values generated using the pair wise sequence alignment program EMBOSS Needle, that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

“Substitution” means that an amino acid residue is replaced by another amino acid residue. Preferably, the term “substitution” refers to the replacement of an amino acid residue by another selected from the naturally-occurring standard 20 amino acid residues, rare naturally occurring amino acid residues (e.g. hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid, ornithine, norleucine, norvaline), and non-naturally occurring amino acid residue, often made synthetically, (e.g. cyclohexyl-alanine). Preferably, the term “substitution” refers to the replacement of an amino acid residue by another selected from the naturally-occurring standard 20 amino acid residues. The sign “+” indicates a combination of substitutions. The amino acids are herein represented by their one-letter or three-letters code according to the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (Ile); K: lysine (Lys); L: leucine (Leu); M: methionine (Met); N: asparagine (Asn); P: proline (Pro); Q: glutamine (Gln); R: arginine (Arg); S: serine (Ser); T: threonine (Thr); V: valine (Val); W: tryptophan (Trp) and Y: tyrosine (Tyr). In the present document, the following terminology is used to designate a substitution: L238A denotes that amino acid residue (Leucine, L) at position 238 of the parent sequence is changed to an Alanine (A). A132V/I/M denotes that amino acid residue (Alanine, A) at position 132 of the parent sequence is substituted by one of the following amino acids: Valine (V), Isoleucine (I), or Methionine (M). The substitution can be a conservative or non-conservative substitution. Examples of conservative substitutions are within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

This disclosure is not intended to be limited to the scope of the particular forms set forth, but is intended to cover alternatives, modifications, and equivalents of the variations described herein. Further, the scope of the disclosure fully encompasses other variations that may become obvious to those skilled in the art in view of this disclosure. The scope of the present invention is limited only by the appended claims.

1: A method of synthesizing a polynucleotide having a predetermined sequence capable of forming a G4 structure, the method comprising the steps of:
(a) providing, attached to a synthesis support, initiators each with a free 3′-hydroxyl; and
(b) repeating in a reaction mixture including the synthesis support, until the polynucleotide is formed, cycles of (i) contacting under elongation conditions the initiators or elongated fragments having free 3′-O-hydroxyls with a 3′-O-blocked nucleoside triphosphate and a template-independent polymerase so that the initiators or elongated fragments are elongated by incorporation of a 3′-O-blocked nucleoside triphosphate to form 3′-O-blocked elongated fragments, and (ii) deblocking the elongated fragments to form elongated fragments having free 3′-hydroxyls, wherein the reaction mixture for elongating the initiators or elongated fragments comprise polyC oligonucleotides capable of forming duplexes with regions of the polynucleotide.

2: The method according to claim 1 wherein said initiators comprise said polyC oligonucleotides.

3: The method according to claim 1, wherein said synthesis support further comprises said polyC oligonucleotides attached thereto.

4: The method according to claim 1, wherein said reaction mixture comprises polyC oligonucleotides in solution whenever said elongated fragments are G4-prone polynucleotides.

5: The method according to claim 1, wherein said polynucleotide is an RNA and wherein said template-independent polymerase is a poly(A) polymerase or a poly(U) polymerase or variant thereof.

6: The method according to claim 5, wherein said 3′-O-blocked nucleoside triphosphate is a 3′-O-azidomethyl-ribonucleoside triphosphate.

7: The method according to claim 1, wherein said polynucleotide is a DNA and wherein said template-independent polymerase is a terminal deoxynucleotidyltransferase (TdT) or variant thereof.

8: The method according to claim 7, wherein said polyC oligonucleotide has a length in the range of from 2 to 20 nucleotides.

9: The method according to claim 7, further including a step of cleaving said polynucleotide from said synthesis support.

10: The method according to claim 7, wherein said 3′-O-blocked nucleoside triphosphate is selected from the group consisting of 3′-O-(2-nitrobenzyl) nucleoside triphosphate, 3′-O-allyl nucleoside triphosphate, 3′-O-amine nucleoside triphosphate, 3′-O-azidomethyl nucleoside triphosphate, 3′-O-(2-cyanoethyl) nucleoside triphosphate, and 3′-O-propargyl nucleoside triphosphate.

11: The method of claim 10, wherein said 3′-O-blocked nucleoside triphosphate is a 3′-O-azidomethyl nucleoside triphosphate.

12: The method of claim 10, wherein said 3′-O-blocked nucleoside triphosphate is a 3′-O-amine nucleoside triphosphate.

13: A kit for synthesizing a polynucleotide of a predetermined sequence using a template-free polymerase comprising a synthesis support having attached thereto initiators comprising polyC oligonucleotides.

14: The kit according to claim 13, wherein said polynucleotide is a polydeoxyribonucleotide and wherein said template-free polymerase is a terminal deoxynucleotidyltransferase or variant thereof.

15: The kit according to claim 13, wherein said polynucleotide is a polyribonucleotide and wherein said template-free polymerase is a poly(A) polymerase or a poly(U) polymerase or variant thereof.

16: The kit according to claim 13, wherein said synthesis support comprises a population of microparticles.

17: The method according to claim 2, wherein said synthesis support further comprises said polyC oligonucleotides attached thereto.

18: The method according to claim 2, wherein said polynucleotide is a DNA and wherein said template-independent polymerase is a terminal deoxynucleotidyltransferase (TdT) or variant thereof.

19: The method according to claim 3, wherein said polynucleotide is a DNA and wherein said template-independent polymerase is a terminal deoxynucleotidyltransferase (TdT) or variant thereof.

20: The kit according to claim 14, wherein said synthesis support comprises a population of microparticles.
